(19) United States
(12) Patent Application Publication
(10) Pub. No.: US 2018/0020973 A1
(43) Pub. Date: Jan. 25, 2018

(54) PROSTHETIC SOCKETS WITH SENSORS

(71) Applicant: LIM INNOVATIONS, INC., SAN FRANCISCO, CA (US)

(72) Inventors: Garrett Ray HURLEY, San Francisco, CA (US); Andrew C. PEDTKE, San Francisco, CA (US); Nathaniel C.R. SMITH, San Francisco, CA (US); Ankur DAS, Oakland, CA (US); Jesse Robert WILLIAMS, San Francisco, CA (US); David C. COHEN, Oakland, CA (US); Ryan Masato SATCHER, San Francisco, CA (US); Loren Maxwell BROCK, Sebastopol, CA (US)

(21) Appl. No.: 15/656,540

(22) Filed: Jul. 21, 2017

Related U.S. Application Data
(60) Provisional application No. 62/364,930, filed on Jul. 21, 2016.

(51) Int. Cl. A61B 5/00; A61F 2/80; A61F 2/60; A61F 2/76

(52) U.S. Cl. CPC A61B 5/4851; A61F 2/76; A61F 2/80; A61F 2/60; A61F 2002/7635; A61F 2002/704

(57) ABSTRACT

A system and method are described for profiling a distribution of forces transferred from the body weight and the residual limb of a wearer of a prosthetic socket through the socket. The system may include a prosthetic socket, a sensor network comprising multiple sensors coupled with the prosthetic socket in a pattern defining multiple internal regions within the prosthetic socket, and a processor coupled with the sensor network and configured to receive sensed data from the sensor network, divide the sensed data into groups corresponding to the multiple internal regions within the prosthetic socket, and process the sensed data to provide force distribution profile data corresponding to the force distribution profile.

### CROSS-REFERENCE TO RELATED APPLICATIONS

This application claims the benefit of U.S. Provisional Patent Application No. 62/364,930, filed on Jul. 21, 2016, which is titled, “Prosthetic Sockets That Are Sensor Enabled to Provide Data for Clinical Use and Mechanical Adjustments,” and which is hereby incorporated by reference.

The present application is also related to the following patents and pending applications: U.S. Pat. No. 8,978,224, entitled “Modular Prosthetic Sockets and Methods for Making Same,” filed on Nov. 13, 2012; U.S. patent application Ser. No. 14/213,788, entitled “Modular Prosthetic Sockets and Methods for Making and Using Same,” filed Mar. 14, 2014, published as U.S. Patent Publication No. 2014/0277584, and now abandoned; U.S. Pat. No. 9,468,542, entitled “Prosthetic Socket and Socket Liner with Moisture Management Capability,” filed on Jun. 20, 2014; U.S. patent application Ser. No. 14/659,433, entitled “Modular Prosthetic Sockets and Methods for Making Same,” filed on Mar. 16, 2015, published as U.S. Patent Application No. 2015/0190252, and now abandoned; U.S. Provisional Patent Application No. 62/275,546, entitled, “Prosthetic Socket That is Sensor Enabled to Provide Data for Clinical Use and Mechanical Adjustments,” filed on Jan. 6, 2016; and U.S. Provisional Patent Application No. 62/334,791, entitled “Prosthetic Support Device for an Osseointegrated Femoral Abutment,” filed on May 11, 2016. All of the above-referenced patents and applications are hereby incorporated by reference, in their entireties, into the present patent application.

### TECHNICAL FIELD

This application is related to prosthetic sockets for use on residual limbs of amputees. More specifically, the application is related to prosthetic sockets with sensors.

### INCORPORATION BY REFERENCE

All publications and patent applications identified in this specification are hereby incorporated by reference to the same extent as if each such individual publication or patent application were specifically and individually indicated to be so incorporated by reference.

### BACKGROUND

Prosthetic limbs for the upper and lower extremities typically include a residual limb socket, an alignment system, and a distal prosthetic component, such as a knee, foot, arm, or hand. The prosthetic socket is the interface between the user's body and the distal prosthetic components. To fulfill its role as an effective and practical interface, the socket needs to fit onto the residual limb and support it well, it needs to effectively suspend onto (or “hold onto”) the residual limb, and it needs to provide a stable and aligned support for the distal prosthetic components. All of these functions are important, but fitting the socket very well to the residual limb has been a long-standing challenge for prosthetic socket technologies.

Even with more modern techniques of socket construction, one continuing challenge with prosthetic sockets is that the volume of any patient's residual limb changes continually, for example with weight gain, recovery from injury, muscle loss or gain, amount of activity of the patient, etc. These continual volume changes pose a serious constraint on the ability of any fixed-form laminated plastic socket to provide a fit that is fully satisfactory over time. In many instances, it takes a significant amount of time to make a prosthetic socket for a patient. In those cases, the initial socket might not fit the patient well, even from the very beginning. Even when the socket does initially fit well, the patient's residual limb almost always undergoes size and shape changes over time, and the initial socket then no longer fits the patient. With most currently available sockets, whenever significant changes in the residual limb occur an entirely new socket must be made. Wearing an ill-fitting socket is generally extremely uncomfortable and can even do permanent damage to the residual limb.

Therefore, it would be desirable to have improved prosthetic sockets and methods for making and adjusting the fit of such sockets. It would be even more ideal to have a system for monitoring the fit of a socket on a patient, to enable the patient or a caregiver to adjust the socket as needed.

### BRIEF SUMMARY

Prosthetic sockets act as an interface between the residual limb and more distal prosthetic components. As such, prosthetic sockets are critically important for in the overall functionality of limb prosthetic devices. As discussed above, for a prosthetic socket to function properly, it must fit extremely well, and the fit of the socket must be maintained through what are often very frequent and significant size and shape changes in the residual limb. Furthermore, the prosthetic socket must fit well during motion, for example with a lower limb socket during walking. This further complicates the prosthetic socket fit equation.

This application describes various embodiments of a prosthetic socket, a socket system, and methods for making and using the socket and the system, all of which involve one or more sensors on the socket for sensing various parameters, such as the forces placed on the prosthetic socket by the patient's residual limb and the socket. Sensed parameter data may be processed and analyzed and may be used by the patient, a caregiver and/or any other user to better understand the way the prosthetic socket fits and interacts with the patient's residual limb and in some cases to help modify the socket to improve the fit of the socket on the residual limb. One example of a sensed parameter is the force (or forces) placed on the socket by the residual limb while the patient is moving, stationary, etc.

In one aspect of the present disclosure, a system for profiling a distribution of forces applied to a prosthetic socket by a residual limb of a wearer of the socket may include a prosthetic socket, a sensor network and a processor. The prosthetic socket may include a proximal portion, a longitudinal portion including multiple struts, a distal portion including a distal base coupled with distal ends of the multiple struts, and an adjustment member coupled with the proximal portion, the longitudinal portion and/or the distal portion, configured to adjust the prosthetic socket to alter a force distribution profile within the prosthetic socket. The sensor network may include multiple sensors coupled with the prosthetic socket in a pattern defining multiple internal regions within the prosthetic socket. The processor is coupled with the sensor network (wirelessly or via a wired connection) and is configured to receive sensed data from the sensor network, divide the sensed data into groups corresponding to the multiple internal regions within the prosthetic socket, and process the sensed data to provide force distribution profile data corresponding to the force distribution profile.

In some embodiments, the processor is directly attached to the prosthetic socket. Alternatively, the processor may be separate from, and wirelessly coupled with, the prosthetic socket. In some embodiments, the processor is housed in a controller configured to allow the wearer of the prosthetic socket or another user to control at least one feature of the prosthetic socket. In one embodiment, the processor may include a microprocessor directly attached to the prosthetic socket and configured to receive the sensed data and wirelessly transmit the sensed data and an off-socket processor separate from the prosthetic socket and configured to receive the sensed data from the microprocessor, divide the sensed data into the groups corresponding to the multiple internal regions within the prosthetic socket, and process the sensed data to provide the force distribution profile data. In some embodiments, such a microprocessor may perform initial processing of the sensed data to provide processed sensed data before transmitting to the off-socket processor. In various embodiments, the off-socket processor may be positioned in a location such as but not limited to a computer application on a smart phone or other smart device, a tablet computer, a laptop computer, a desktop computer, a computer server or the cloud.

Any suitable sensor may be included in the sensor network, such as but not limited to a force sensor, a strain gauge, a Hall sensor, a flex sensor, a proximity sensor, a GPS, a flex sensor, a 3-axis accelerometer, a 3-axis gyroscope, and/or a 3-axis magnetometer. In some embodiments, the multiple internal regions include a proximal region corresponding to the proximal portion of the prosthetic socket, a longitudinal region corresponding to the longitudinal portion of the prosthetic socket, and a distal region corresponding to the distal portion of the prosthetic socket. In some of these embodiments, each of the proximal region, the longitudinal region and the distal region is further divided into four sub-regions: a mediolateral region; a medioposterior region; a lateroanterior region; and a lateroposterior region. In some embodiments, the multiple internal regions further include an ischial seat region. In some embodiments, the longitudinal region is further divided into an upper longitudinal region and a lower longitudinal region.

In some embodiments, the proximal portion of the prosthetic socket may include a brim member coupled with proximal ends of at least some of the struts. In some embodiments, the proximal portion of the prosthetic socket may include an ischial seat member. Optionally, the force distribution profile data may include multiple percentages of force applied by the residual limb to the prosthetic socket over the multiple internal regions. The force distribution profile may also describe force distribution through each of the multiple struts. In some embodiments, the force distribution profile describes force distribution in a central path through a distal end of the residual limb and a peripheral path through the longitudinal portion of prosthetic socket.

In some embodiments, the processor may be further configured to compare the force distribution profile data with a desired force distribution profile stored in a computer memory of the system and provide the wearer or another user with comparison data. For example, such comparison data may include an alert when the force distribution profile is outside of a predetermined range of desired force distribution profile data. In some embodiments, the adjustment member may be an adjustable hinge configured to be fixed at a desired angle. Alternatively, the adjustment member may be an adjustable height ischial seat member mounted on a proximal end of one of the multiple struts. In some embodiments, the adjustment member may include at least one tensioning band coupled with the multiple struts and at least one tension adjustment member attached with each of the at least one tensioning bands. In other embodiments, the adjustment member may include a motorized adjustable closure mechanism configured receive a command signal from the processor and automatically adjust the prosthetic socket based on the command signal. Optionally, the adjustable motorized closure mechanism may be further configured to maintain a tension in the prosthetic socket within a predetermined tension range. In some embodiments, the adjustment member includes a hinge mechanism configured receive a command signal from the control unit and automatically adjust the prosthetic socket based on the command signal.

Optionally, the sensor network may further include at least one off-socket sensor configured to be attached to the wearer of the prosthetic socket at a location separate from the prosthetic socket. The system may also optionally include a control unit coupled with the prosthetic socket, where the processor is housed within the control unit. In an alternative embodiment, the system may include a control unit that is physically separate from the prosthetic socket, where the processor is housed within the control unit and the sensors are wirelessly coupled with the control unit.

In another aspect of the present application, a method for generating force distribution profile data for a prosthetic socket on a residual limb of a wearer of the socket may first involve sensing forces applied to multiple predefined regions of an inner surface of the prosthetic socket by the residual limb, using a sensor network attached to the prosthetic socket. The sensor network generally includes multiple sensors disposed in the predefined regions. Next, the method involves transmitting sensed data from the sensor network to a processor coupled with the sensor network and processing the sensed data with the processor. The processing step includes grouping the sensed data into multiple groups corresponding to the predefined regions and generating force distribution profile data for the prosthetic socket, based on the sensed data and the multiple groups.

Optionally, the method may further involve providing the force distribution profile data to a user. Additionally or alternatively, the method may also include automatically adjusting the prosthetic socket, based on the force distribution profile data. In such an embodiment, the method may optionally also involve comparing the force distribution profile data to a desired force distribution profile, and the step of automatically adjusting the prosthetic socket is based at least in part on the comparing step. In some embodiments, the method also includes sensing an acceleration of at least a portion of the prosthetic socket, using the sensor network, and generating an acceleration distribution profile for the prosthetic socket, based on the sensed acceleration. Alternatively, the method may involve sensing a position of at least a portion of the prosthetic socket, using the sensor network, and generating a position distribution profile for the prosthetic socket, based on the sensed position.

In some embodiments, the prosthetic socket may include three or more struts, and generating the force distribution profile may involve comparing amounts of force in each of the struts. In some embodiments, the prosthetic socket is a transfemoral prosthetic socket including four struts, where one of the struts is a medial-posterior strut, and generating the force distribution profile involves comparing an amount of force delivered through the medial-posterior strut with amounts of forced delivered through the other three of the four struts.

In some embodiments, the prosthetic socket includes a microprocessor, and the transmitting step involves transmitting the sensed data from the sensor network to the microprocessor and transmitting the sensed data from the microprocessor to the processor, where the processor is located separately from the prosthetic socket. Such a method may optionally further involve conducting initial processing of the sensed data at the microprocessor before transmitting to the processor. The processor may be located off of the prosthetic socket and be coupled wirelessly with the sensors of the sensor network, according to some embodiments. In other embodiments, the processor may be directly attached to the socket and may be coupled wirelessly or via wire(s) with the sensors.

In some embodiments, transmitting the sensed data may involve transmitting a location identifier from each of the multiple sensors. Some embodiments of the method may also involve displaying the force distribution profile data on a display device. For example, the display device may be a controller that is separate from the prosthetic socket. The processor may be housed in the controller, for example. The method may also include providing an alert on the controller when the force distribution data falls at least partially outside of a predetermined range of desired force distribution data.

In some embodiments, the method also includes adjusting tension in the prosthetic socket, using a motorized tensioning mechanism attached to the socket, based at least in part on the force distribution profile data. In some embodiments, the tension is adjusted automatically. Some embodiments may involve automatically adjusting at least one characteristic of the prosthetic socket to adjust the force distribution profile data toward a desired force distribution profile. In some embodiments, adjusting the force distribution profile data may involve moving a force distribution profile longitudinally within a length of the prosthetic socket. In some embodiments, adjusting the force distribution profile data involves moving a force distribution profile within a cross sectional anterior-posterior/lateral-medial grid within the prosthetic socket. In some embodiments, adjusting the force distribution profile data involves moving a force distribution profile comprises actuating a hinge mechanism within the prosthetic socket.

Examples of force distribution profile data include but are not limited to a distribution of forces impinging on the prosthetic socket in relation to a central longitudinal axis of the socket, an absolute level of force applied by a distal end of the residual limb to the prosthetic socket, and a relative fraction of a total force applied by a distal end of the residual limb to the prosthetic socket. In some embodiments, the method may also include testing the prosthetic socket on the residual limb and determining a desired force distribution profile for the socket and the residual limb. Testing the prosthetic socket, for example, may include placing the prosthetic socket on the residual limb and making initial adjustments to one or more mechanical features on the prosthetic socket.

In any given embodiment, the method may involve repeating the sensing, transmitting and processing steps as many times as desired. For example, the steps may be repeated continuously over a period of time or at set time intervals.

In another aspect of the disclosure, a non-transitory computer readable medium for use on a computer system may contain computer-executable programming instructions for performing a method as described immediately above. In various embodiments, the method may include any or all of the features and/or steps described above.

These and other aspects and embodiments will be described in more detail below, in reference to the attached drawing figures.

### BRIEF DESCRIPTION OF THE FIGURES

FIG. 1 is a schematic representation of the distribution of force as it flows from the body weight of a wearer of a prosthetic socket through the wearer's residual limb and the socket, according to one embodiment (the residual limb and socket are not shown);

FIG. 2 is a front, partially transparent view of a residual limb and part of a pelvis of a patient wearing a transfemoral prosthetic socket, illustrating the distribution of force from the body weight of the patient and the residual limb to and through the socket, according to one embodiment;

FIG. 3 is a perspective, partially transparent view of a residual limb of a patient wearing a transtibial prosthetic socket, illustrating the distribution of force from the body weight of the patient and the residual limb to and through the socket, according to one embodiment;

FIG. 4 is a perspective, partially transparent view of a residual limb of a patient wearing an osseointegrated abutment support socket, illustrating the distribution of force from the body weight of the patient and the residual limb to and through the socket, according to one embodiment;

FIG. 5A is a side view of a modular smart transfemoral prosthetic socket with sensors positioned at various locations, according to one embodiment;

FIGS. 5B and 5C are side and bottom views, respectively, of a proximal brim of the transfemoral prosthetic socket of FIG. 5A;

FIG. 5D is a side view of a distal portion of the transfemoral prosthetic socket of FIG. 5A, according to one embodiment;

FIGS. 5E and 5F are a partially transparent side view and a bottom view, respectively, of a distal cup of the transfemoral prosthetic socket of FIG. 5A;

FIGS. 5G and 5H are perspective and exploded views, respectively, of an adjustable height ischial seat of the transfemoral prosthetic socket of FIG. 5A;

FIGS. 6A and 6B are exploded and side views, respectively, of a modular smart transfemoral prosthetic socket with multiple sensors and a data collection and transmission system, according to one embodiment;

FIGS. 7A and 7B are top views of a modular data collection and transmission system flat pattern for use with a smart prosthetic socket, according to one embodiment;

FIG. 8 is an exploded view of the modular data collection and transmission system of FIGS. 7A and 7B;

FIG. 9A is a diagram illustrating a system and method for monitoring a prosthetic socket, using a mobile computing device and a server, according to one embodiment;

FIG. 9B is a diagram illustrating a system and method for monitoring a prosthetic socket, using a mobile computing device, a server and an additional computing device, according to another embodiment;

FIG. 10 is a diagram illustrating data flow between various components of a prosthetic socket monitoring system, according to one embodiment;

FIG. 11 is a block diagram illustrating a smart prosthetic socket monitoring system, including a sensor interface, a smart application and a mobile application interface, according to one embodiment;

FIG. 12A is a flow diagram illustrating a method for monitoring smart prosthetic socket performance data and manually adjusting the smart prosthetic socket, according to one embodiment;

FIG. 12B is a flow diagram illustrating a method for monitoring smart prosthetic socket performance data and automatically adjusting the smart prosthetic socket, according to an alternative embodiment;

FIG. 13 is a diagram illustrating data flow between multiple components of a sensor-enabled smart prosthetic socket system, according to another embodiment;

FIG. 14 is a side view of a lower half of a patient's body, wearing a smart prosthetic socket attached to distal prosthetic components, illustrating communication between the distal prosthetic components and the sensor-enabled smart prosthetic socket, according to one embodiment;

FIGS. 15A-15D are illustrations of a smart phone with various graphical user interface pages for an application for use with a sensor-enabled smart prosthetic socket system, according to one embodiment;

FIGS. 16A-16D are additional illustrations of the smart phone of FIGS. 15A-15D, illustrating additional pages of the graphical user interface, according to one embodiment;

FIGS. 17A and 17B are cross-sectional top and posterior views, respectively, of a patient's residual limb, illustrating a method of profiling force distribution through a transfemoral prosthetic socket, according to one embodiment;

FIGS. 17C-17E are posterior, lateral and anterior views, respectively, of a lower extremity of a human, illustrating the lower extremity anatomy;

FIG. 17F is a front view of a transtibial residual limb;

FIG. 17G is a front view of clinically relevant regions of a transtibial residual limb;

FIG. 17H is a posterior view of a transtibial residual limb;

FIG. 17I is a posterior view of clinically relevant regions of a transtibial residual limb, according to one embodiment;

FIG. 18A is a diagram of a key for identifying prosthetic socket regions represented within a dashboard image of a sensor-enabled smart prosthetic socket, according to one embodiment;

FIG. 18B is a top view of a sensor-enabled smart prosthetic socket, as outlined in FIG. 18A, according to one embodiment;

FIGS. 19A and 19B are two side views of a transfemoral prosthetic socket, with two struts removed for clarity, showing socket regions according to a sensor distribution, according to one embodiment;

FIG. 20A is a diagrammatic representation of a user interface dashboard image of an optimal distribution of force that represents an optimal socket state, according to one embodiment;

FIG. 20B is a diagrammatic representation of a user interface dashboard image of a sampled force distribution that deviates from the optimal socket state, according to one embodiment;

FIG. 20C is a diagrammatic representation of a user interface dashboard image of the differences between an optimal distribution of force (per FIG. 20A) and a sampled distribution of force (per FIG. 20B), according to one embodiment;

FIG. 20D is a diagrammatic representation of a user interface dashboard image of the correction needed to resolve the differences shown in FIG. 20C, according to one embodiment;

FIG. 20E is a diagrammatic representation of a user interface dashboard image of an optimal distribution of force that represents an optimal socket state, as in FIG. 20A;

FIG. 20F is a diagrammatic representation of a user interface dashboard image of a sampled force distribution that deviates from the optimal socket state, according to one embodiment;

FIG. 20G is a diagrammatic representation of a user interface dashboard image of the differences between an optimal distribution of force (per FIG. 20E) and a sampled distribution of force (per FIG. 20F), according to one embodiment;

FIG. 20H shows a user interface dashboard image of the corrections need to resolve the differences shown in FIG. 20G, according to one embodiment;

FIG. 21 is a diagrammatic representation of a user interface dashboard or graphical display of a user's weight bearing distribution, according to one embodiment;

FIG. 22A is an exploded view of a modular smart transfemoral sensor-enabled prosthetic socket with a data collection and transmission system that uses wireless communication and an automated closure system, according to one embodiment;

FIG. 22B is a close-up perspective view of the automated closure system of FIG. 22A.

FIG. 22C is a side, cross-sectional view of the automated closure system of FIGS. 22A and 22B;

FIG. 23 is an exploded view of a modular smart transfemoral sensor-enabled prosthetic socket, with a different automated closure system, according to an alternative embodiment;

FIG. 24A-24D are front views of four different low-profile automated closure mechanisms for an automated smart prosthetic socket, according to four alternative embodiments;

FIG. 25 is an exploded view of a modular smart transfemoral prosthetic socket with a sensor-enabled pneumatic bladder system, according to one embodiment;

FIGS. 26A and 26B are perspective and flattened views, respectively, of the sensor-enabled pneumatic bladder system of FIG. 25;

FIG. 27A-27C are exploded perspective views of a material layup for fabrication of a sensor-enabled pneumatic bladder system for a smart prosthetic socket, according to one embodiment;

FIG. 28 is a perspective, silhouette view of a pneumatic bladder of a smart prosthetic socket changing shape as it inflates, according to one embodiment;

FIG. 29 is an anterior cross-sectional view of a residual limb inside a modular smart prosthetic socket, including sensor-enabled pneumatic bladders, according to one embodiment;

FIG. 30A and FIG. 30D are detailed perspective views of a portion of the prosthetic socket of FIG. 29, showing how one of the sensor-enabled pneumatic bladders is integrated into the socket, according to one embodiment;

FIGS. 30B and 30C are perspective and cross-sectional views, respectively, of the sensor-enabled pneumatic bladder of FIGS. 30A and 30D;

FIGS. 31A and 31B are top views of a lateral textile sling with a pneumatic bladder system, for incorporation into a smart prosthetic socket, according to one embodiment;

FIG. 31C is a top view of a medial textile sling with a pneumatic bladder system, for incorporation into a smart prosthetic socket, according to one embodiment;

FIG. 31D is a top view of a textile sling system, in which the lateral and medial textile slings of 31A-31C are coupled together, according to one embodiment;

FIGS. 32A and 32B are a perspective view of a residual limb (FIG. 32A) and a top view of a custom-sized textile sling with pneumatic bladder system (FIG. 32B) made to fit the residual limb and for incorporation into a smart prosthetics socket, according to one embodiment;

FIGS. 32C and 32D are a perspective view of a residual limb (FIG. 32C) and a top view of a custom-sized textile sling with pneumatic bladder system (FIG. 32D) made to fit the residual limb and for incorporation into a smart prosthetics socket, according to another embodiment;

FIGS. 33A and 33B are cross-sectional views of a residual limb and a sensor-enabled pneumatic bladder system used to manage volume change and/or intentionally vary the amount of pressure on the residual limb, according to one embodiment;

FIG. 34 is a perspective view of a prior art prosthetic foot and ankle with a pneumatic evacuation system that uses kinetic energy of ambulation to evacuate air out of the system;

FIG. 35 is a perspective view of a miniature pneumatic pump used to inflate and/or deflate a sensor-enabled pneumatic bladder system of a smart prosthetic socket, according to one embodiment; and

FIGS. 36A-36C show movement of a residual limb and a prosthetic socket during a stride and accommodation for the movement by an integrated smart bladder system, according to one embodiment.

### DETAILED DESCRIPTION

As mentioned above, this application describes various embodiments of a prosthetic socket, a socket system, and methods for making and using the socket and the system, all of which involve one or more sensors on the socket for sensing various parameters. For example, two types of prosthetic socket sensors are force and motion sensors. Sensed force and motion data may be used to evaluate a patient's experience and clinical outcome with the socket and in some embodiments to make adjustments to the socket to improve fit. Adjustments may be made automatically, for example using microprocessors in communication with actuators, manually by the patient or prosthetist, or a combination of automatically and manually. In addition to sensing forces and motion, a host of other types of sensors may be included in a prosthetic socket, according to various alternative embodiments. For example, some sockets may sense temperature or humidity within a prosthetic socket, pulse, blood pressure, electrocardiogram (ECG or EKG) signals and/or electromyographic (EMG) signals.

In this application, sensor-enabled prosthetic socket embodiments and the hardware and software of the system used to support and interact with the socket may be referred to generally as embodiments of a “smart socket.” The totality of data reported by prosthetic socket sensors at a specific time on smart socket embodiments may be referred to as the physical “state” of the socket. The smart socket state may also be time stamped or tagged in conjunction with qualitative data, clinician data, or other data. The socket state as well as qualitative data, clinician data, or other data may be referred to as “total input data.”

Many, if not all, of the embodiments of prosthetic socket systems described herein may be incorporated into (or adapted for use in) any of a number of types of prosthetic sockets. Examples of the types of prosthetic sockets that may be especially amenable to the socket sensors and sensor systems described herein are the modular, strut-based prosthetic sockets that have been described previously by the assignee of the present application. Examples of such prosthetic sockets are described in detail in U.S. Pat. Nos. 8,978,224, 9,044,349 and 9,468,542, and U.S. Patent Application Pub. Nos. 2014/0277584 and US 2016/0058584, all of which are incorporated herein by reference. Other examples will be discussed below, and these examples are not meant to be limiting.

The above-referenced publications describe a prosthetic socket assembled from modular components that include a distal base, multiple longitudinal struts indirectly or directly connected to the distal base, and one or more brim elements, each of which is connected to at least one strut. In some prosthetic socket embodiments, a strut connector element intervenes between the distal end of a strut and a connection site on the distal base, or the struts connect indirectly. In addition to these basic structural components, embodiments of the prosthetic socket may also include a distal cup, one or more encircling bands and/or tensioning elements or closure mechanisms that are applied around the struts and or the brim elements, as well as soft good elements, such as sleeves that are placed over the struts. Ancillary components associated with the prosthetic socket may include socket liners that protect the residual limb and can remove accumulated moisture. Sensors can be integrated into the smart socket in various different ways, between or around various different parts, and within various different parts of the modular assembly.

Examples of sensors that may be used in various embodiments of the prosthetic sockets described herein include but are not limited to sensors that detect and quantify force, torque, load, and/or pressure in many different ways, including in-line load cells, pancake load cells, rotary shaft torque sensors, and flush threaded pressure sensors. Other examples of sensors include motion sensors (such as accelerometers, inclinometers, gyroscopic sensors), magnetic field sensors, global positioning sensors, altimeters, thermometers, moisture sensors, photo sensors, and cameras. Timing and/or clock functions may also be integrated into the sensors. In some embodiments, multiple positioning or gyroscopic sensors can be configured as RF transceivers that communicate with a single receiver, thereby enabling triangulation to add another dimension to positioning of the patient and/or the socket in space.

Other vital sign or biometric sensing devices may be included in various embodiments, such as a heart rate sensor, blood flow sensors, blood oxygen sensors, blood pressure sensors, EMG sensors, and other heath monitoring sensors integrated into modular components to monitor limb health and/or the general health of the patient. In some instances, some or all of the sensing devices used in a given prosthetic socket system may be off-the-shelf units and/or not substantially integrated within the prosthetic socket itself. Such sensors, however, may be included in the prosthetic socket system as a whole, and data acquired by these sensors may be integrated downstream in a data processing and analysis function, to build out a more complete clinical picture. The preceding enumerated sensors are intended merely as non-limiting examples, and various embodiments may include any sensor that can deliver clinically useful information regarding the status of the patient, the socket and/or the environment within or near the socket.

Other sensed data may come from third party or associated sources, such as distal components, a smart prosthetic gel liner, a smart prosthetic liner or sock, a third party smart phone application (such as a dietary application or sleep application), a third party data collecting device (such as a smart watch or fitness tracker) and/or the like. These third party or associated sources may also provide electrical power or other resources. For example, a smart socket may couple with the microprocessor prosthetic knee or power knee to collect data, share data, and share electrical power. This may improve accuracy and quantity of data, may allow for new automated response mechanisms, such as data from the smart socket regarding how the knee or other distal components should respond to the environment, and reduce the number of devices the user needs to plug in or replace batteries for.

The modular aspect of some of the sensor-enable prosthetic socket embodiments described herein refers to the fact that components of these sockets are available in a range of sizes and shapes and can be assembled into a socket and swapped out for other parts as necessary, while still fitting together via common connection elements. Of particular significance in embodiments of sensor-enabled sockets, as described herein, is the fact that each connection site is a site across which force (e.g., pressure, tension, torque) may be transmitted when the socket is worn by a patient engaged in the activities of daily living. Tensioning or closure elements that provide circumferential adjustments typically include two connection portions, such as a buckle, ladder-lock or loop-lock arrangement connection, or any suitable reversible or adjustable tensioning or closure element connection mechanism.

Accordingly, in some embodiments, force sensors may be advantageously positioned at these sites where prosthetic socket components connect to another component, or within a two-part component, such as a tensioning element. Force sensors and other sensors may also (or alternatively) be located at any advantageous position on the hard structure of the prosthetic socket, or at any suitable position within soft goods or fabric portions of the socket that are made to couple with any type of prosthetic socket. The sensors may also be integrated into many different types of prosthetic sockets, such as vacuum formed thermoset or thermoplastic prosthetic sockets that are non-modular, modular prosthetic sockets that are fabricated from one or more inventoried components, thermoset or thermoplastic modular prosthetic sockets, or prosthetic sockets made of other materials, such as elastomers, urethane, alloys and/or phase changing alloys. Non-modular laminated prosthetic sockets may require a dummy or spacer element that is integrated into the mold during fabrication then removed to allow for integration of the sensors. Motion sensors may be advantageously positioned at relatively distal sites within the prosthetic socket, inasmuch as movement tends to increase with increasing distance from the patient's hip.

Data that are collected from the sensors may be relayed to a remote or onboard microprocessor unit for immediate or future use and/or stored or saved remotely or onboard the prosthetic socket. Remote devices, such as a computer at the patient's home or at a clinic, may be configured to receive data. Mobile phones or dedicated mobile receivers or transceivers may be carried by the patient, and such devices may further transmit the data to a remote server or a local computer.

In some embodiments, sensed prosthetic socket data may be exploited to make immediate or nearly immediate mechanical adjustments or other forms of adjustment, such as changes to a pneumatic bladder, temperature change of the prosthetic socket, change to a phase changing material such as a phase changing alloy or phase changing fluid, and/or changes with thermoformable materials within the socket. Some prosthetic socket embodiments have sites where mechanical adjustments can be made that affect the sizing or shape-based fit of a socket to the residual limb of a patient. These adjustments may be made manually, either by the patient or by a prosthetist or other caregiver, for example. In other embodiments, the adjustments may alternatively or additionally be made automatically, when sensor data is processed by an associated microprocessor that is, in turn, enabled to appropriately activate an actuator to affect a responsive mechanical adjustment.

Sensed data or the total input data may also immediately or nearly immediately be used for notifications, predictive analysis, and prescriptive information. For example, sensor data can measure the amount of pressure inside regional aspects of the socket, a processor may analyze the sensor data to determine that the pressure in the proximal-medial aspect of the user's transfemoral smart socket is beyond the maximum threshold based on qualitative data, such as previous comfort scores and targets the user has set for their daily use. A notification can immediately be sent to the user's smart phone, with prescribed or recommended manual adjustment.

In some embodiments, sensed data may be used on a longer-range time line but may still result in one or more mechanical adjustments, such as those just described. Longer-range time refers to a data collection period of more than a single day, typically a duration of a week or more. The longer-range time line may allow for sensor data that is more highly resolved and dependable, and it may also allow the processor to analyze patterns in the data. Data and patterns may analyzed with software, mathematical equations, and algorithms built from clinical experts and users who are knowledgeable on extracting clinically relevant metrics from the total input data. Mechanical adjustments made in response to these types of accumulated data are typically made be a prosthetist, but a patient may participate as well.

As mentioned above, in some embodiments, a prosthetic socket may automatically adjust in response to sensed socket data, via one or more mechanical adjustment actuators on the prosthetic socket. Microprocessor-enabled automatic control of adjustable points in the prosthetic structure may occur in sites, by way of example, where angles or tensioning elements occur. U.S. Pat. No. 8,978,224, incorporated herein, describes several examples, including adjusting the angle of struts with respect to the base, adjusting the length of a telescopically enabled strut, adjusting the circumferential tension applied either to struts or brim elements, and automatically adjusting air or hydraulic pressure within a pneumatic or hydraulic bladder or camber system.

One type of automatic adjustment actuator that may be used in a sensor-enabled prosthetic socket is a sensor-controlled servomotor or stepper motor integrated into one or more tensioning or closure elements of the socket (or multiples of same). For example, servomotors may be disposed at two sites on the socket, across a joining element that connects two portions of a tensioning element. The effect of appropriately configured servomotors is to loosen or tighten tensioning elements, depending on sensed data and predetermined responses according to where the data land within predetermined ranges.

Another example is sensor-controlled servomotors that adjust the take-off angle of struts with respect to the distal base. The distal ends of struts attach to the distal base, and a separate intervening strut connector may be present in some embodiments. The takeoff angle affects the volume encompassed by the socket, particularly in the distal region of the socket. Tension or force sensors disposed within the socket can sense outward pressure that the residual limb is directing against the struts. The effect of the appropriately configured servomotors, by way of adjusting the takeoff angle, is to expand or contract the volume enclosed by the struts, depending on sensed data and predetermined responses according to where the data land within predetermined ranges.

Another example of an automatic adjustment mechanism for a prosthetic socket is inflatable and deflatable hydraulic or pneumatic bladders, which may be positioned within the smart socket along with an expulsion valve and a small compressor or pump, such as a mini-diaphragm air pump. For example, fabric strut and polyurethane bladders may be positioned on or along the struts of a prosthetic socket or between struts or against a socket liner. Sensors can be positioned on a strut sleeve, such that they are subjected to pressure that exists between the socket structure and the residual limb. Sensors may also be integrated into pneumatic bladders that are between struts within the brim or within cross-connectors, other textile components, the distal base, the distal cup, or other aspects of the socket. These sensors within the pneumatic or hydraulic bladders, which may communicate with a microprocessor, which may be operatively coupled to a bladder pump and vent or valve, can monitor pressure on the limb and respond in real time by inflating or venting, depending on sensed data and predefined commands. For example, the system may operate to maintain pressures/forces within the prosthetic socket within predefined ranges. Any type of bladder(s) may be used in such embodiments—e.g., pneumatic, air, fluid, gel or the like. Air or fluid bladders may also include baffles or geometric chambers designed to provide a specific pattern or motion or direction of force and pressure distribution when inflated or deflated. This direction-specific capability within the bladder can be designed to provide increased biomechanical control or can selectively increase inflation or deflation in regions where volume change typically applies. Any type of sensor-enabled bladder(s) described herein may be referred to as a “smart bladder” or “smart bladder system”.

In some embodiments, bladders may have force sensors integrated into them, such that the bladder can measure forces directly against the residual limb. A desired pressure range may be set by the user or clinician, for example via a smart phone application and smart socket microprocessor, and may be used to inflate or deflate as needed to maintain a certain amount of force or pressure. In some embodiments, the amount of force or pressure may be deliberately changed over time. Automated adjustment, in these bladder-based embodiments, would also increase or decrease the volume within the socket and may thereby automatically adjust for volume change of the residual limb. Inflation and deflation may also be used in cases where volume is consistent but the desired amount of pressure within the socket differs per amount of control that is needed per activity. For example, when a user sits down, the pressure may decreased, and when the user runs, the pressure may be increased, to provide more force needed to control the prosthesis while running.

Any type of smart bladder(s) described may also be used in such embodiments to maintain desired amounts of pressure per specific region of the socket, as set by the user or clinician, and/or intentional variation or oscillation of pressure per time spent using the prosthesis.

One example of the above smart bladder system is that the user may use a user interface application on a smart phone to establish that they want the pressure within the proximal-medial aspect of the socket and distal-lateral aspect of the socket to be maintained at an average pressure of 20 pounds, while the proximal-lateral aspect of the socket and distal-medial aspect of the socket are to be maintained at an average pressure of 10 pounds. This pressure would be automatically maintained as the user experiences changes in volume or changes their activity, and software is used to analyze or adjust pressure thresholds, average pressures, and/or peak pressure variance. A user may also use the described smart bladder system to reduce or increase pressure per activity. For example, the user may set the system to relax pressure to zero when they are sitting for more than five seconds. The smart socket would know the user is sitting, because data processing software includes pattern recognition of sensors that correspond with sitting. Settings may also be set for pounds per square inch (psi) within the bladder as opposed to pressure exerted onto the residual limb by the bladder.

Sensors that would enable these options in setting would be pressure sensors built within the layers of the bladder walls and/or pressure transducers built into the bladder system at the valve or at another location to test the pressure within the bladder. For some users, 5 pounds of pressure or 3 psi may be their high pressure setting, whereas others may select pressures of 20 pounds or 15 psi or greater for their high setting. For some users, 2 pounds of pressure or 2 psi may be their low pressure setting where as others may select for pressures of 5 pounds or 5 psi or lower for their low setting. These are merely examples, however, and any suitable combination of pressure settings may be used/selected in a given embodiment.

Another example of the above smart bladder system is that the user may use their user interface application on their smart phone to establish that they want the pressure within their smart bladder system to oscillate between 0 and 20 pounds 4 times at regular intervals throughout the day or upon request or allowance after notification via smart phone application. This oscillation or variation may be used to promote blood flow, other bodily fluid circulation, and/or other comfort of biological benefit.

Similar to the way these smart bladder systems can be used within a smart socket described herein, under-actuated mechanical joint systems may also be used to biomechanically control the residual limb, add comfort, and/or accommodate volume change. An under-actuated mechanical joint system is a system that provides minimal actuation or automation to control motion or response across multiple joints. For example, one cable may pass on the outside of a series of joints, while another cable may pass on the inside of multiple joints, and a motor or motors can actuate the motion of those cables to control the motion of multiple joints at once.

Another type of an automatic adjustment mechanism for a prosthetic socket is artificial muscle technology that may be used for automated response to sensed data or the total input data. This technology may be used in conjunction with the smart bladder systems described above or in other ways, such as shortening or lengthening a closure or biomechanical control mechanism.

Another type of automatic adjustment is height or length adjustment of one or more elements within the prosthetic socket, such as an automated height adjustment of the patellar tendon area of a transtibial prosthetic socket or the ischial seat area of a transfemoral socket. Some prosthetic socket embodiments have an ischial seat that is positioned at a medial and posterior aspect of the socket. The ischial seat engages the ischium of the patient and can bear weight that would otherwise be born by the residual limb, particularly the distal end of the residual limb. To bear weight effectively, the height of the ischial seat from the distal base of the socket should be properly adjusted. Small differences in height can have a large effect on the patient's comfort. For these reasons, it is advantageous for the ischial seat height to be adjustable. Pressure absorbed through the ischial seat is thus a useful indicator. A pressure sensor positioned such that it senses pressure through the ischial seat, and informed by a microprocessor, can be configured to drive movement of a linear actuator that adjusts the height to the ischial seat. Bladders as described above may also be used for this adjustment. Pneumatic or hydraulic cylinders may also be used for this type of adjustment and may also include a given range of shock absorption within the system so that the height may vary within a range that is predetermined by the user or clinician. For example, a user may set their ischial seat to maintain an average pressure of 30 pounds and to allow movement of plus or minus 5 mm from the selected baseline height or length. Adjustments may be made to accommodate for volume change or for changes in activity. For example, when a user sits, the ischial seat may automatically lower when it is not needed. Alternatively, when a user is walking and the pressure on their distal end gets past their self-selected socket threshold and, due to previous total input data, it is determined that raising the seat is more effective and comfortable for the user than tightening the closure system or tensioner, the seat can be automatically raised to reduce pressure on the bottom.

As mentioned above, any of a wide variety of sensors and any suitable combination of sensors may be incorporated into and/or used in conjunction with a prosthetic socket in providing a smart socket system as described in this application. In addition to using sensor-derived data for automatic adjustments of the socket, examples of which were just described, the data may also be used to guide manual adjustments of the socket and/or for general information about the patient's interaction with the socket and may include any of the above describe mechanisms or adjustment methods in a manual fashion. The general information may be used for any suitable purpose, such as in guiding the care of that patient, in developing clinical studies involving multiple patients, in collecting and analyzing data for multiple patients to help improve prosthetic socket manufacturing and/or use, or the like. What follows is a description of non-limiting examples of the types of clinically valuable information that may be derived from sensors incorporated into or used in conjunction with prosthetic sockets.

Pedometer devices may be used to track step count, the duration and times during the day when socket is worn, and the time it is not worn. Metrics of walking or running, such as speed and distance, may be tracked. Similarly, sedentary or standing time may be tracked. GPS location and altimeters may be used to capture details of location, distance, and terrain covered during walking or riding in a vehicle. Pattern recognition algorithms may be used to acquire data, characterize individual routines, such as donning or doffing the socket, and allow these routine activities to be tracked specifically. Pattern recognition may also be combined with gait analysis, so that gait features can be identified from data alone. Moisture levels within the space between the socket and a liner and/or in the space between the liner and the residual limb can be tracked with moisture sensors. Pressure in critical areas within the socket, as well as overall tightness of the socket, can be tracked. In a closely related function, overall volume of the residual limb may be tracked. In particular embodiments, blood pressure and pulse rate may be tracked. Photosensors or cameras can monitor skin color changes in critical or sensitive areas of the residual limb. Notifications, alerts, alarms, or instructions can be associated with any sensor (e.g., pressure, tension, moisture, temperature), with a range of values defined as normal and a range (or upper and/or lower limits, for example) defined as concerning. Changes in inductance may be used to track changes in circumference of the residual limb, and circumferences throughout the residual limb may be used to mathematically estimate the overall volume of the residual limb. These are merely examples of some of the parameters that may be sensed and measured with various embodiments of the sensor-enabled prosthetic socket systems described herein.

In some embodiments, data of the type described above may be used for making quick adjustments to the prosthetic socket and/or for creating awareness of a clinical issue immediately. The data may also be used by a prosthetist or healthcare provider who is following the patient over a longer period of time, and the longer-term data may also be used by the prosthetist or other caregiver to make socket adjustments, replace socket components, provide advice to the patient and/or the like. For example, the prosthetist may use the data to advise the patient regarding daily routines, patterns of activity, or levels of tension or pressure in the socket that may be optimal all over the socket and/or in specific regions of the socket.

In some embodiments, the sensor-enabled prosthetic socket system may include a user interface and data processing module or software, such as an application that may be used on a cell phone or mobile data capture device. The processing module may receive and transmit sensed data from the socket and in some embodiments may have broader uses as well. For example, the application can enable direct messaging between patient and prosthetist, or between a prosthetist and a researcher, or between the patient and the company that manufactures, markets, or distributes the prosthetic socket, or between the patient and patient interest or advocacy organizations. An application may have a questionnaire that can be filled out daily, or periodically, that captures the patient's subjective and qualitative experience. Such data can be usefully associated with the sensed data for cause and effect analysis by a prosthetist or researcher, or used by the software itself to identify patterns or adjust parameters as the increased data allows for improved learning about user needs and requirements. A software application may also provide connection directly to social media or to a private user group, thus providing peer and social support to the patient, in addition to more strictly clinical communications.

One example of a way in which sensed data from a prosthetic socket may be used relates to a scoring system to evaluate clinical outcomes for patients fitted with prosthetic sockets and such outcomes mapped against data sets obtained from patients. Such a system may evaluate the following criteria, for example:

(1) Comfort: For example, the 10-point socket comfort scoring system of Hanspal may be used (Disabil Rehabil 25(22):1278-80, 2003).

(2) Daily usage: For example, an accelerometer or pedometer may be used, and the data points may be time used per day and distance traveled or step count per day.

(3) Functionality: For example, Stevens, et al., (The Academy Today 5(1), February 2009) provides a comprehensive list of clinical relevant outcome measurements.

(4) Efficiency: This outcome relates to the efficiency of satisfactorily situating a patient in the socket with respect to the patient's time and effort, and the efficiency with respect to healthcare costs. This metric may be determined by tracking the number of clinical visits required from initial evaluation for a new socket to final socket delivery and also follow-up visits requested by a patient over a three-month period after initial socket delivery for socket related issues.

(5) Mobilization: This metric refers to the duration of time between (a) a patient's first evaluation for a prosthetic socket and the clinical decision to proceed with a new socket, and (b) delivery of a satisfactory socket to the patient for the initiation of ambulatory socket use. As a basic metric, mobilization can be expressed as the number of days from evaluation to ambulation. It relates primarily to operational efficiency of the clinic and turn-around factors specific to the socket. The metric can also be enriched by inclusion of other clinical factors or with details associated specifically with the initial trauma or limb condition, the level of amputation, etc.

A number of different assessments of a prosthetic socket and its interaction with the residual limb may be made, according to various embodiments. Examples include the following. (1) Changing residual limb volume, during the day or over the course of several days, reflecting changes in fluid pressure in the body. Compensating or correction intervention may include changes in tension of a tensioning belt (or belts) on the socket. (2) Changing residual limb volume over the longer-term volume, such as weeks or months, reflecting overall muscle and bone volume. Compensating or correction intervention may include changes in placement of a tensioning belt (or belts) on the socket. (3) Change in overall fit of the prosthetic socket: change in relative weight on pressure sensors (seat-strut-distal loading goes from 70%-20%-10% to 55%-30%-15%). (4) Activity level, for example as monitored by a motion sensor. Changes in activity level, per cumulative motion data, can be analyzed in conjunction with various measures of prosthetic socket “fit.” (5) Gait change, for example as monitored by a motion sensor. Example: change in average lateral motion during a step.

These preceding variables and the associated tracking data may be analyzed by a prosthetist that is following the patient. In response to the data, a prosthetist has a range of options, including making minor mechanical adjustments, changing ranges and responses that are automated, replacing the prosthetic socket or particular components, etc. Further, in some instances, patients have impaired cognition, perception, or communication, in which case sensor-based data can be particularly valuable.

In one embodiment, tension sensors are arranged at the junction of two connecting portions of a tensioning or closure element, such as a strap, belt, or webbing. Data from such tensioning sensors may be processed by a processor of the system to provide a profile of residual limb volume as it fluctuates during the day or as it changes gradually over weeks and months. Alternatively, change in inductance measurements can also be used for circumferential and volume measurements with a high degree of accuracy. Such an embodiment may include one or more tension sensors, such sensor(s) enabled or configured with features such as any of the options listed below.

(1) Feedback and sensing module (the device) may either be integrated into or in-line with a belt or webbing material that is typically under tension for use in prosthetics.

(2) Device may have onboard processing and feedback capabilities, wireless communication capabilities, and is battery-powered.

(3) Device may include a combination of sensors integrated into the belt or webbing module.

(4) Device may use raw sensor values to gather metrics related to wear time of prosthesis.

(5) Device may use raw sensor values to gather metrics related to activity level of a patient wearing the prosthetic device. Motion sensing of activity to be determined used to quantify activity levels.

(6) Device may use raw sensor values to gather metrics related to tightness (tensile force) present in belt or webbing of prosthesis.

(7) Device may use raw sensor values to gather metrics related to spatial orientation of prosthesis.

(8) Raw sensor values collected by device may be transformed into metrics that are useful for patients and care providers by way a series of custom algorithms developed for each prosthetic application.

(9) Device can deliver real time feedback to users via wireless streaming live data to a smart phone or computer.

(10) Device can operate in two modes of operation: (1) continuous data collection with non-real-time wireless streaming to a smart phone or computer (used to collect patient data over an approximate week time frame), or (2) continuous data collection with live wireless streaming to smart phone or computer (used for real testing and data collection experimentation.

(11) Device has modular belt attachment to swap out belt or interchange module with various prosthetic devices.

(12) Software (web and mobile app) that can display long-term trends, averages, and provides feedback to patients and/or care providers based up the data collection from the device. Data and relevant information is based on custom algorithms above stated prosthetic algorithms.

(13) Device may have on board general-purpose input/output ports to power and collect data from sensors external to the device casing. These include but are not limited to force, temperature, pressure, and humidity.

(14) The device may collect data from the above stated external sensors, store the data on-board, and transmit the data via a wireless data connection to a smart phone or computer.

Within the prosthetic socket, one or more of the following measurements are enabled by one or more sensors integrated into the prosthetic device; moisture, forces including pressure and tension, strain, shear, position of the socket and residual limb, geographic location of the user, relative movement/motion including changes in vertical position, lateral, and, horizontal position of the residual limb, user, or socket. Sensors within or adjacent to the socket or on the residual limb may also enable measurements of; residual limb volume, changes in residual limb volume, skin integrity or skin injury of the residual limb or contralateral limb, muscle or connective tissue integrity of the residual limb or contralateral limb, muscle or connective tissue activity of the residual limb or contralateral limb, as well as biometric measurements such as temperature, blood flow, heart rate, and blood glucose level/blood sugar.

One embodiment may include a modular addition that covers the inside and/or outside a prosthetic socket with instrumented textiles capable of sensing force in discrete regions. The textile force sensors are composed from a thin flexible piezoresistive film and conductive textile leads. Additionally, a 9 axis IMU is fastened to the modular addition. The array of force sensors and IMU are connected to a microcontroller for onboard data processing. This microcontroller parses the raw data from the previously mentioned sensors to a simplified list of gait metrics. This data is stored on onboard electronic flash memory, before being uploaded to the phone via Bluetooth Low Energy communication.

The mobile application acts as both a data transfer and user interaction tool. The phone communicates with the socket-side microcontroller/data transmission unit/battery via Bluetooth Low Energy, receiving the calculated gait metric data and validating their successful transfer. The mobile app then uploads the data to the cloud via cellular network or Wi-Fi.

Independent of the socket, the mobile app allows the user to interact with their data and access other social features. The phone retrieves past data from the cloud on user request. The amputee can then view their past data through visual display elements. These elements may include highlights of excellent or poor gait performance or activity, charts tracking gait progress and socket fit changes across time, comparison to self-determined goals, or others. Amputees may also upload other data manually, such as photos of painful areas or injuries, socket comfort score questionnaires, or abnormalities in socket function. The mobile app also contains a variety of social features for amputees to interact with each other, which may include chat capabilities with other amputees, activity comparisons and challenges with friends, tutorials and educational videos, or other social features.

The back-end server hosts a secure database that stores patient data. This server receives incoming data from the mobile app, both manually uploaded and transmitted from the socket. This server runs additional algorithms to further identify useful metrics from the collected data. The total input data is used across different users to establish standards or normality of data and then the individual's data can be compared to that of established standards to identify deviations or potential issues that may lead to injury or sub-optimal performance. The user entered targets as well as qualitative and quantitative data collected into the mobile application is also compared to the current socket state in order to identify any deviations or trends that may be lead to a targeted optimal state or to pain. The mobile application then sends data back to users on the web or mobile app for viewing. The server stores patient, prosthetist, and account info in addition to the collected sensor and app data.

The web-app has similar functionality to the mobile app for amputees, while allowing different permissions and functions for different types of users. Amputees can still retrieve data from the cloud, view their data history, manually input data, and access the various social features found on the mobile app. Other parties using the mobile-app will have access to different data and features, depending on their roles: e.g. prosthetists may access their patients' data, communicate securely with patients, communicate with other healthcare team members, and contact paying parties.

Qualitative data collected is collected through a smart phone application including; self-reported overall socket comfort score/socket comfort, regionally specific self-reported socket comfort, self-reported socket control or biomechanical control of the their residual limb within the prosthetic socket, suspension rating or how intimately the prosthesis stays on their residual limb, how tight or snug the socket feels around their residual limb, negative events such as skin breakdown or skin injury, muscle or connective tissue injury, reduced range of motion, falls, and discomfort as well as positive events such as increased range of motion, target socket states, goals, and presets, clinician or healthcare professional adjustments, clinician or healthcare professional goals, presets and target socket states.

In addition to input from sensors within and around the prosthetic socket and qualitative data collected through a smart phone application; additional input may be gathered through independent or third party applications, fitness trackers, and user interfaces. This may include a third party smart phone application that collects data on calories consumed or a fitness tracker that records the amount of sleep. Data transfer from a third party smart phone application is facilitated or aided by an application program interface (API) which is a set of routines, protocols, and tools for building software applications. An API specifies how software components should interact. Additionally, APIs are used when programming graphical user interface (GUI) components as it relates to routines, protocols, and parsing information created and acquired with in that systems network. The API allows the smart socket system to real time scrape a user's data from a third party application to the smart phone application and user interface associated with the described device and method.

Metrics described herein are referring to conclusions or meaningful results that are extrapolated or condensed from raw data that comes from one or more measurements or sensors. Metrics extrapolated from the measured data described above include; gait, weight distribution, user activity, and qualitative results. More specifically, weight distribution within the socket, abnormal gait patterns, gait speed, cadence, changes in direction, gait obstacles successfully navigated (such as a curb or set of stairs), changes in vertical distance, number of steps, distance of gait, duration of prosthetic use, gait distribution or comparisons of phases of gait such as stance phase vs. swing phase, changes in cadence, changes in activities (standing vs. sitting vs. walking), range of motion of the prosthetic limb, suspension movement on the residual limb (vertical movement between the residual limb and the prosthesis/socket), tension in the closure system vs. comfort in the socket, congruency or relative motion between the prosthetic socket and the residual limb, changes in volume, changes in tissue integrity, changes in tissue density, changes temperature, changes in muscle activity, calories burned (with prosthetic use which can be calculated from any metrics such as changes in vertical distance, gait distance, changes in direction, number of steps, gait speed, and other data), gait symmetry (percentage of time spent on prosthetic side vs. contralateral side or right and left for a bilateral amputee) and biological changes such as changes in blood sugar and changes in temperature vs. changes in activity calculated from biometric measurements. Qualitative result metrics may include; percentage of time spent in target socket weight distribution, percentage of step count goal accomplished, differences in quantitative data and qualitative reporting such as the difference in measured movement and reported perception of control, and longitudinal changes in their reported socket comfort score. These metrics can be used to improve care or service to the user, can facilitate safety and monitoring capabilities, can help facilitate insight and better service from healthcare professionals, and can confirm or refute usage for insurance payment.

Such an embodiment of a base model may include one or more of the following specific metrics: steps; stance versus swing; gait speed (ground speed); distance of gait; cadence (steps per time); K-Level (a combination of gait obstacles successfully navigated (such as a curb or set of stairs), changes in cadence, changes in vertical distance, gait distance, and changes in activities); socket state (pressure distribution per region, overall pressure, and pressure relationships); time of use (used vs not used, standing vs sitting vs walking); and/or geographic location. Such an embodiment of an expanded model may additionally include one or more of the following specific metrics: abnormal gait patterns; changes in direction; gait obstacles successfully navigated (such as a curb or set of stairs); changes in vertical distance; range of motion of the prosthetic limb; suspension movement on the residual limb (vertical movement between the residual limb and the prosthesis/socket); congruency or relative motion between the prosthetic socket and the residual limb; tension in the closure system vs comfort in the socket; residual limb volume, changes in residual limb volume; skin integrity or skin injury of the residual limb or contralateral limb; muscle or connective tissue integrity of the residual limb or contralateral limb; muscle or connective tissue activity of the residual limb or contralateral limb; calories burned (with prosthetic use which can be calculate from an metrics such as changes in vertical distance, gait distance, changes in direction, number of steps, gait speed, and other data); and/or gait symmetry (percentage of time spent on prosthetic side vs contralateral side or right and left for a bilateral amputee). Biometric measurements may include temperature; blood flow; heart rate; and/or blood glucose level/blood sugar. Qualitative result metrics may include: percentage of time spent in target socket weight distribution; percentage of step count goal accomplished; quantitative data and qualitative reporting such as the difference in measured movement and reported perception of control; and/or longitudinal changes in their reported socket comfort score.

What is described as an algorithm or algorithms refers to the set of steps, programming, mathematical formula, or process used to convert input or measured data into predictive analysis, prescriptive analysis, conclusions, or informative derivatives. Put simply, algorithms are used to accomplish valuable or meaningful outputs with the inputs of collected data. One example of an algorithm described is the set of steps needed to identify and send a notification to the user that they may need to tighten their socket brim in order to avoid pain and injury on the end of the residual limb. If the prosthetic socket device described includes an embodiment with an integrated mechanism for automatically adjusting the fit of their prosthetic socket. The primary process of determining what may need to change with the fit can still be applied, then an additional algorithm would be applied to use measured data in and derive an appropriate automated adjustment of the fit, to avoid user pain and discomfort.

A foundation or pool of data of existing research and tests may be generated and used as a body of knowledge or data that can be compared against itself without any users. These standard test and existing research may include Amputee Mobility Predictor (AMPnoPRO) and research on human gait. This data may be used to inform processing and conclusions from data collected from the user and the socket.

After a user is fit with a sensor-enabled prosthetic socket as described herein, baseline measurements may be measured on regular basis and statistical analyses may be conducted on the measurements to establish baseline for each user. The user is also prompted to record their optimal baseline or fit goal or target. This optimal baseline, fit goal, or target becomes a benchmark, where the total input data is saved at that moment as well as leading up to that moment and can then be compared to other states or sets of total input data and used for predictive algorithms, machine learning, prescriptive information, and notifications. Data used for said baseline may include all or any of the types described and may be stamped or tagged by the user, healthcare professional, or authorized family member through a smart phone application that includes a preset or record or comfort rating button, where the user can add qualitative information about their experience while simultaneously recording all sensor inputs.

In some embodiments, healthcare professionals can also set parameters and record goals/target states. These settings can be made along with adjustments to the fit or alignment of the prosthesis and are stored as independent variables from the user settings and can be compared to user settings. Furthermore, the resulting condition or experience of the amputee from these settings can be compared to that of their own settings to determine if the adjustment or alternative setting has been helpful. If the adjustment results in an improvement, supervised learning models will be applied to learn the new parameters or settings. Regression models or support vector machines can be applied to these parameter values. In machine learning, support vector machines (SVMs, also support vector networks) are supervised learning models with associated learning algorithms that analyze data used for classification and regression analysis. Each user's settings and models are individually adjusted per variation in data input.

In one method, the user's health state and mobility state may be calibrated against standard tests, such as Amputee Mobility Predictor (AMPnoPRO), four-square step test (FSST), up-and-go test/L-test, two minute walk test, four minute walk test, and research on human gait. The user may be asked to perform tests, such as the AMPnoPRO, and data can be collected while the user is participating in these tests. This process can be repeated multiple times with different control of fit parameters, and at different time length after the socket has been worn, and take the measurements for each. Conducting and recording the results for these tests is easier and more accurate through use of a smart phone application which can show the user and tester what the user needs to do and can start and stop the test while recording for improved accuracy. Statistical analyses are conducted on the measurements to calibrate the measurements against the actions and against the existing data pool of research conducted using these same tests. Measurements of the contralateral leg may also be used to calibrate results and as input into the predictive model to explain whether limb health is less than ideal. The baseline measurements along with calibration data points may also be used as training data for various machine learning models or methods.

Calibration can also be made by adjusting the socket a known amount, then recording the resultant sensor input, qualitative data via a smart phone application interfacing with the user. For example, the socket closure system or tensioner may be tightened by 3 cm or 50 pounds. of tension force, and the resulting change in pressure distribution and pressure profile as well as changes in the accelerometer and/or other input can be recorded. This may help to calibrate the data and may also be used to understand and set parameters for automated adjustment capabilities. For example, testing and data collection in this way may yield a conclusion that when distal end pressure exceeds 70 pounds. for a given user, increasing closure tension by 50 pounds. results in an average increase of socket comfort score of 40%.

Negative events relating to use of a prosthesis, such as an injury or fall, do occur. The described sensor enabled prosthetic socket and app-based user interface will allow the user, healthcare team, and/or user's family to record negative events when they occur. The recorded data leading up to these negative events become a valuable tool in avoiding future injury. For example, if a user records in their user interface app that they discovered a blister on the proximal-medial aspect of their socket this evening when they took off their prosthesis; the recorded settings from the day showing that the user had 55 pounds. of pressure in the proximal-medial aspect of their residual limb and that usually they have under 50 pounds. This metric of difference can be used to give notifications in pressure, to the proximal-medial aspect of their socket, stating that it has exceeded 50 pounds., and give an adjustment recommendation that has been learned, to move the user towards a target socket state. This can thereby help to avoid future injury and guide the user towards target states. Because there is a potential high variance between different data samples, learning from individual user events in this way and building sets of data across users is required in order to accurately and appropriately identify precursors of negative events and direct a user towards goals.

Negative events such as pain can also be predicted by detecting certain gait patterns that have been associated with pain in independent studies, such as antalgic gait and circumduction. Identifying such patterns in the amputee's gait distribution may help to detect or avoid pain. The frequency and velocity of such patterns in the measurement data can also be used as an indicator for the degree of pain. In order to improve accuracy, the predictive model herein pulls from multiple types of data and uses multiple analytical methods in order to improve accuracy and user or clinical relevance.

Amputees' pain points may vary by the time of day, tasks, and the physiological or dietary variables such as how much sodium the user consumed in the last 24 hours. With this device and method, users can set indicator flags of discomfort before the onset of the pain. These indicator flags are correlated with the input data for that specific time interval and used to build predictive models for pain. These inputs are treated as a binary classification problem (pain, no pain), a multi-class classification problem (such as no pain, light pain, medium pain, pain, extreme pain), or probability estimation problem.

Pain detection capabilities built into these models are also useful for people who have difficulty recognizing or sensing the pain themselves such as young children and/or people with nerve damage. Said methods may be able to drastically improve the user's ability to avoid pain or more precisely identify pain through these machine learning methods.

Detecting and avoiding falls may be made possible by data collected during controlled falls within harness systems and user input after falls have occurred. Sensors can also be used to determine the amount of clearance at the foot and can warn the user that the clearance in swing or the stability in stance is not sufficient. Furthermore, precursors or variables that increase the risk of a fall can be used to notify users of their increased risk. For example, lack of sleep, dehydration, and low caloric intake data that can be collected by a partner API such as Fitbit, or My Fitness Pal, can predict that a user has an increased chance of falling and can notify the user of this risk. This same partner data can also help to determine a likelihood or cause of volume reduction due to dehydration or excessive sodium intake for example.

In addition to comparisons of data for explicit time variables, it is also useful and valuable to longitudinally compare data. In other words, data is analyzed and compared over large periods of time, in addition to comparing data from specific time stamps. This analysis allows for broad conclusions to be drawn such as whether or not a user's activity level and mobility has increased or decreased. Mobility measurements such as AMPnoPRO can also be used as an additional data points to the overall metric of mobility over time.

Given the system can see and determine certain predefined antalgic gait patterns, these can act as an early stage warning system for a clinical expert. By example a user is seen in the system to be repeating a pattern that indicates osteoarthritis. The system would notify the associated specialist of a patient/user and this evaluation would take place in a prosthetist's office. This ability to sense gait patterns and this early stage warning system is an added benefit to patients and health care providers for creating opportunities for clinical intervention to manage potential problems before they get too out of hand, resulting in example of this case a total knee replacement in the sound side or good leg of an amputee.

Mobility improvement or deterioration may happen in a very slow process over long span of time. Very slow changes in measurements as people age or other conditions occur are to be detected by a fine-tuned amputee mobility assessment. This is accomplished by the calibration data obtained earlier, very detailed measurements data over long span, and statistical analysis for detecting shift in data. Users who allow for their data to be released and compared to other users can leverage increased machine learning capabilities through the increased pool of data.

An overall wellness index that takes data from user's daily input on the comfort/pain scale, pressure profile data, accelerometer data, partner API data, clinical metrics, and/or negative events such as antalgic gait, ulcers, or falls if they occur; can be used to track general trends in residual limb and overall wellness. Trending this index can provide a global view of whether the use of the socket is optimal and whether the usage is improving over time.

Qualitative recordings of the experience while simultaneously recording all inputs historically leading up to that time are prompted at regular time intervals and available for the user to enter whenever they desire to self-report information to the system. They are encouraged by the system to report whenever they feel a state change or discomfort they would like for the system to learn or take note of as it relates what their goals may be for comfort, control, tension or suspension. For example, the smart phone application may automatically prompt the user to enter their comfort score and user experience once a week and the user can additionally go to the interface home button and record their experience at any time. The user will also be asked to log any negative events or especially positive events such as a particularly comfortable and functional day on in their prosthetic socket. Predictive algorithms and machine learning principles such as Bayes' Theorem are applied to the described inputs, longitudinal data, cross-sectional data and other types of data in order to yield prescriptive information or notifications that can be useful to the user, healthcare team, researchers, and family.

The total input data for a given user can be typed or profiled into user categories or groups. Patterns can be identified within these groups and algorithms for these groups of similar people with amputation can be more accurate for predictive and prescriptive information, can more accurately and appropriately respond to automated adjustment of the socket, can have an improved feedback significance, and can more appropriately form user networks of similar people with higher likelihood of providing useful psychological and logistical peer support.

According to one embodiment, a method for using sensed prosthetic socket data may involve the following steps: foundational data is created (including user information like age, weight, height, etc); user baseline or targets are established; adjustments or targets may also be set by healthcare professionals; tests or calibration may also be conducted and recorded by healthcare professionals; any negative events are automatically and qualitatively recorded; longitudinal, cross-sectional, and other types of data collected over time are compared and analyzed automatically by the application programming or cloud based computing; qualitative recordings and their subsequent inputs are prompted at regular time intervals and available to the user at any time; and predictive algorithms and machine learning principles such as Bayes' Theorem are applied to yield prescriptive information or notifications that can be useful to the user, healthcare team, researchers, or family.

The steps described above may be repeated multiple times and increased machine learning from previous process cycles can be improved. Some cycles may only be portions of the steps before cycling back through again. For example, the last 4 steps may be repeated for many different cycles then the user decides to recalibrate their targets so then a loop may occur with all steps described.

The data or inputs, analysis of described inputs, conclusions from described inputs or analysis, and/or notifications may be provided to the patient, authorized healthcare professionals, family members, payers, researchers, and/or the like. One way of providing the data is via a computerized user interface, which may also serve, in some embodiments, as a cross-collaborative communication network where data, experience, insights, and other valuable elements can be shared across the network and from one party to another. For example, if a user experiences an ulcer on their residual limb after a trip to the store, they can take a picture of their skin and push it to their doctor, prosthetist, and caretaker along with the total input data recorded from that day or that trip.

Embodiments of the invention are directed toward using sensor-derived information to profile the physical state of a prosthetic socket when worn by a patient. “State”, as used herein, refers to a description of a system in terms of classical mechanics. Sensor-derived information from a prosthetic socket thus refers to any aspect of a prosthetic socket, as worn by a user, which is informative of the mechanics or physical properties of a socket in time and space. The status of forces within and flowing through the socket is an example of what may be broadly understood as the socket's report of what its physical state is, in terms of any of force, tension, acceleration, position, or phase of a material composition.

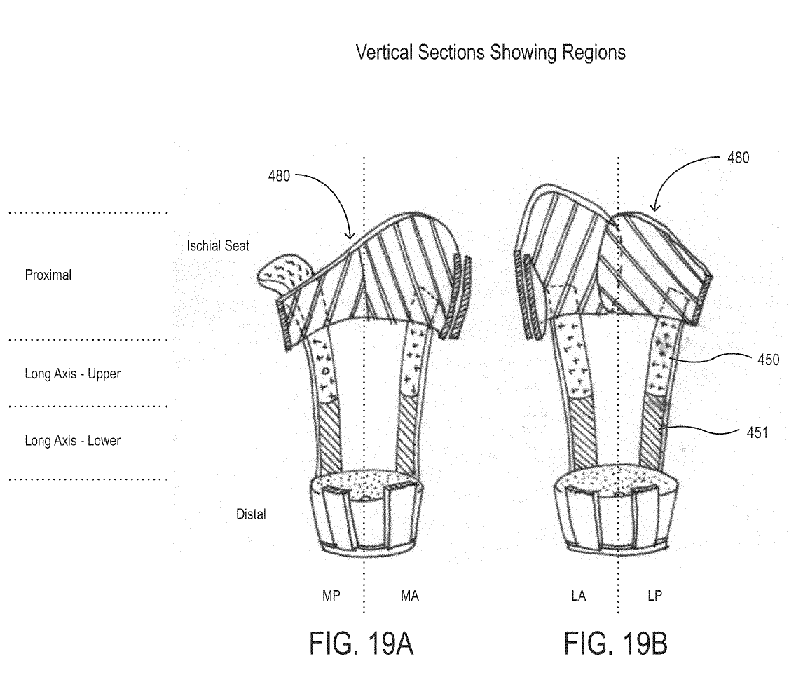

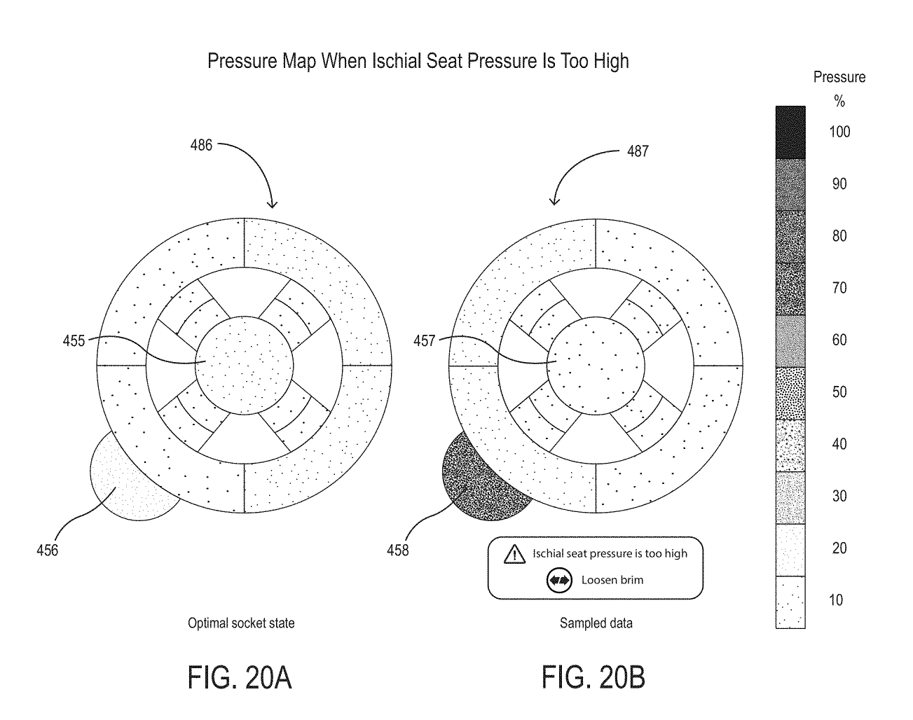

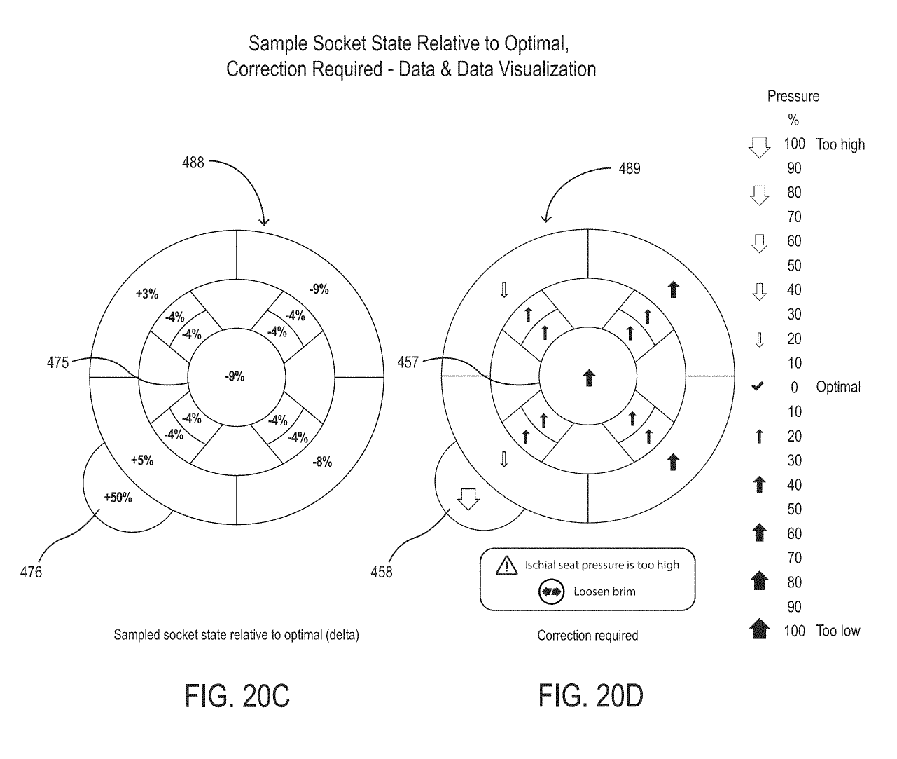

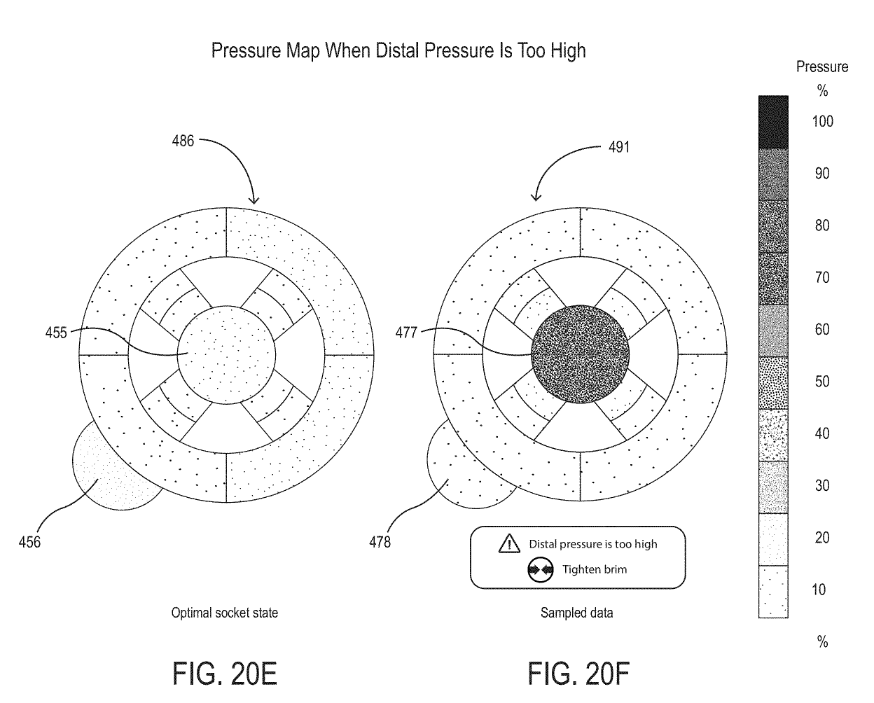

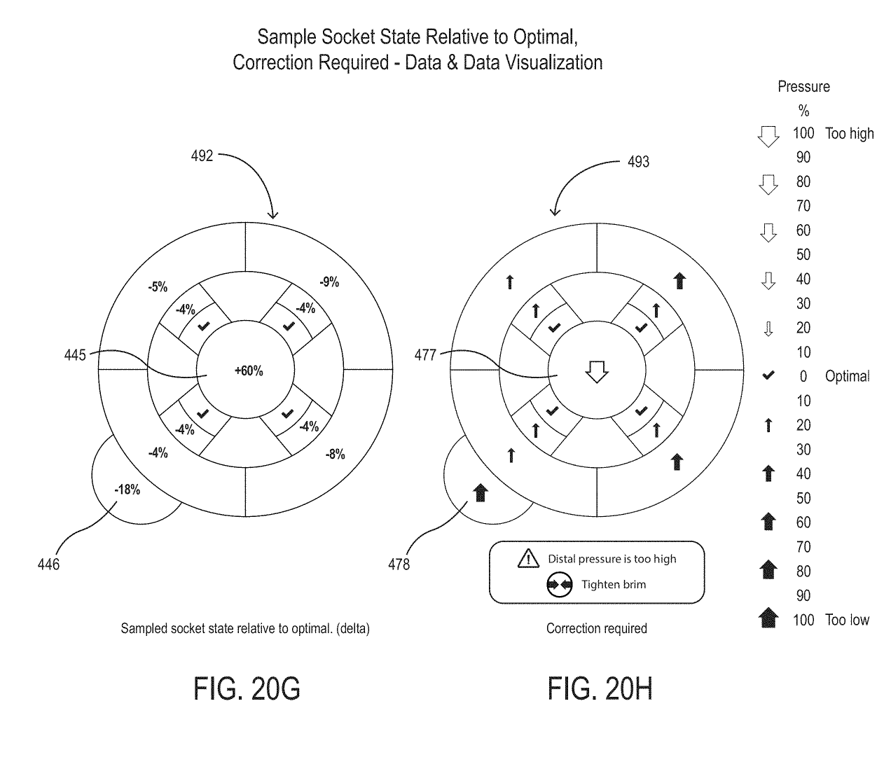

In a basic view of a prosthetic socket as an intermediary device bridging a proximal residual limb to a distal prosthetic limb, embodiments of the invention profile the distribution of force within the prosthetic socket as such force travels from the prosthetic residual limb to the distal prosthetic limb. Examples of sensor-reported data and socket state, as depicted in FIGS. 20A-20H, focus on the distribution of force paths as they flow through the socket, however embodiments of the invention further include reporting, analyzing, regionalizing, and depicting any aspect of physical state, including, merely by way of example, acceleration, tension, position, or material phase.

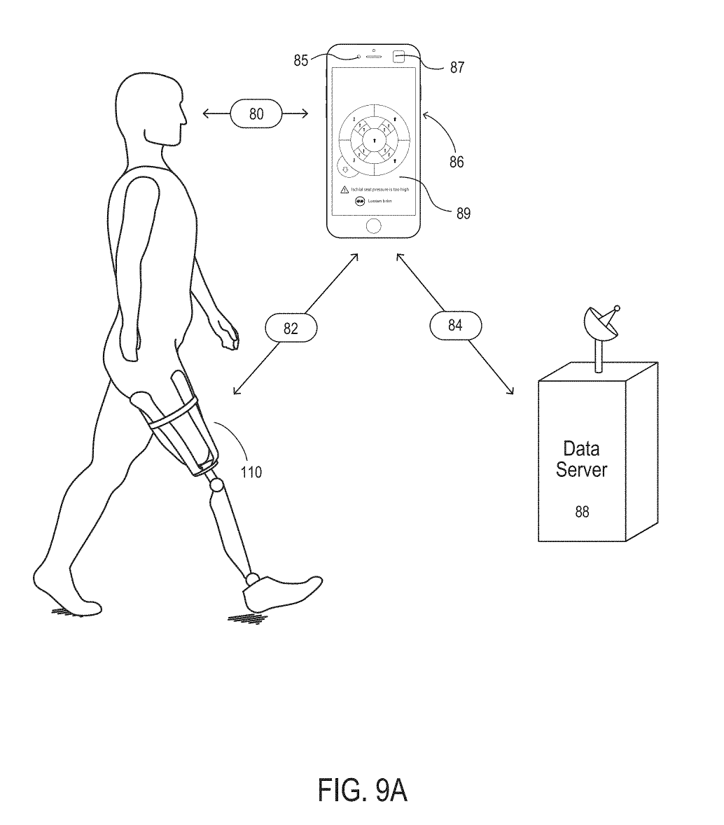

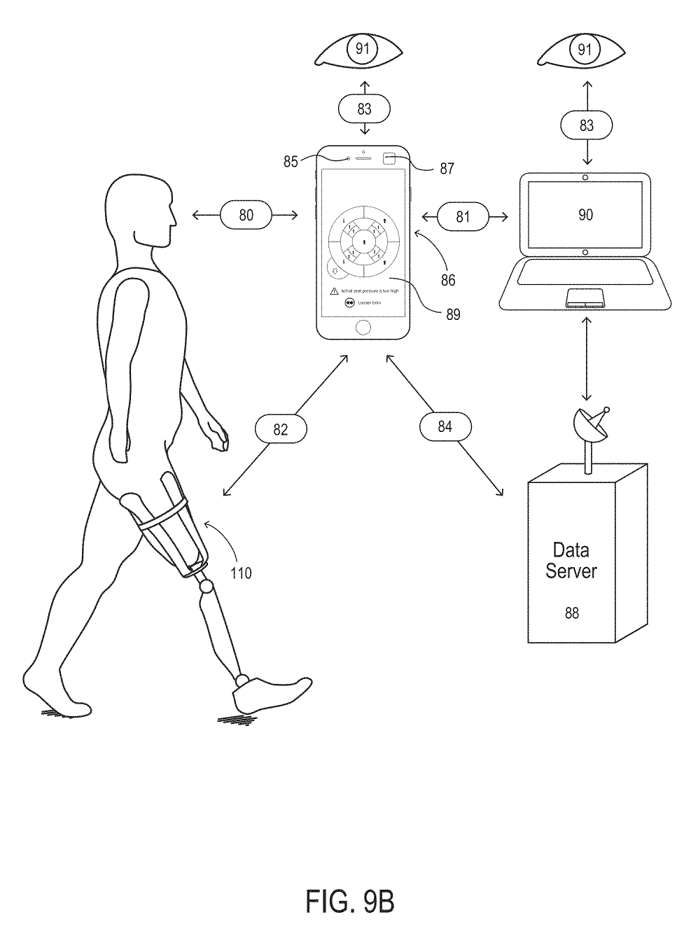

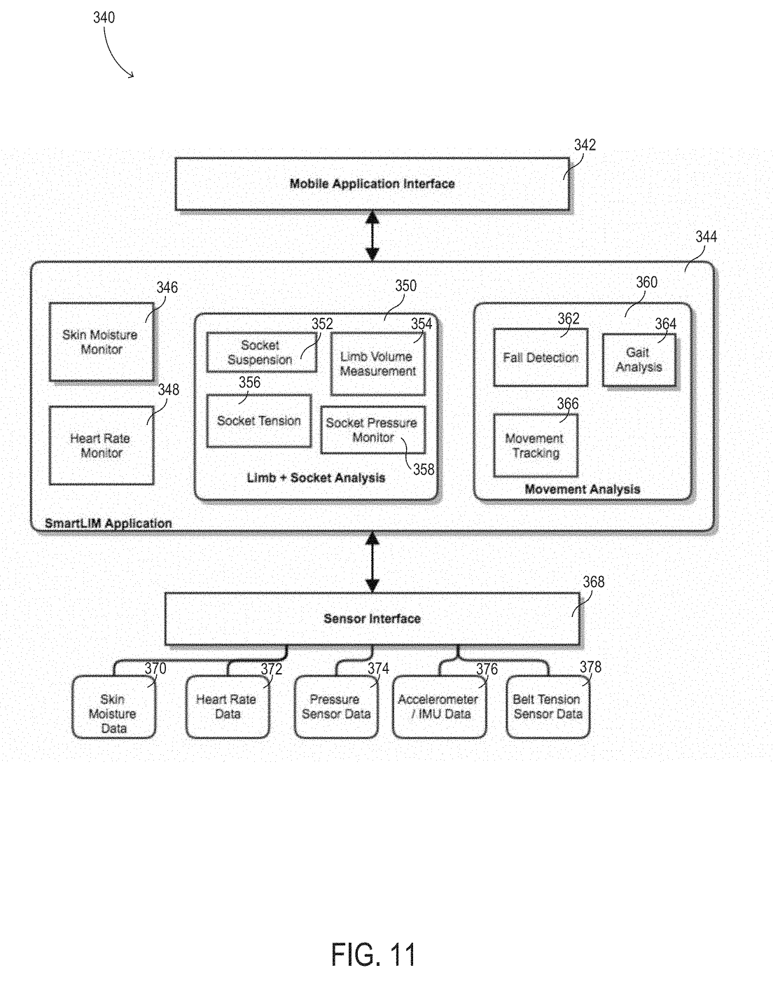

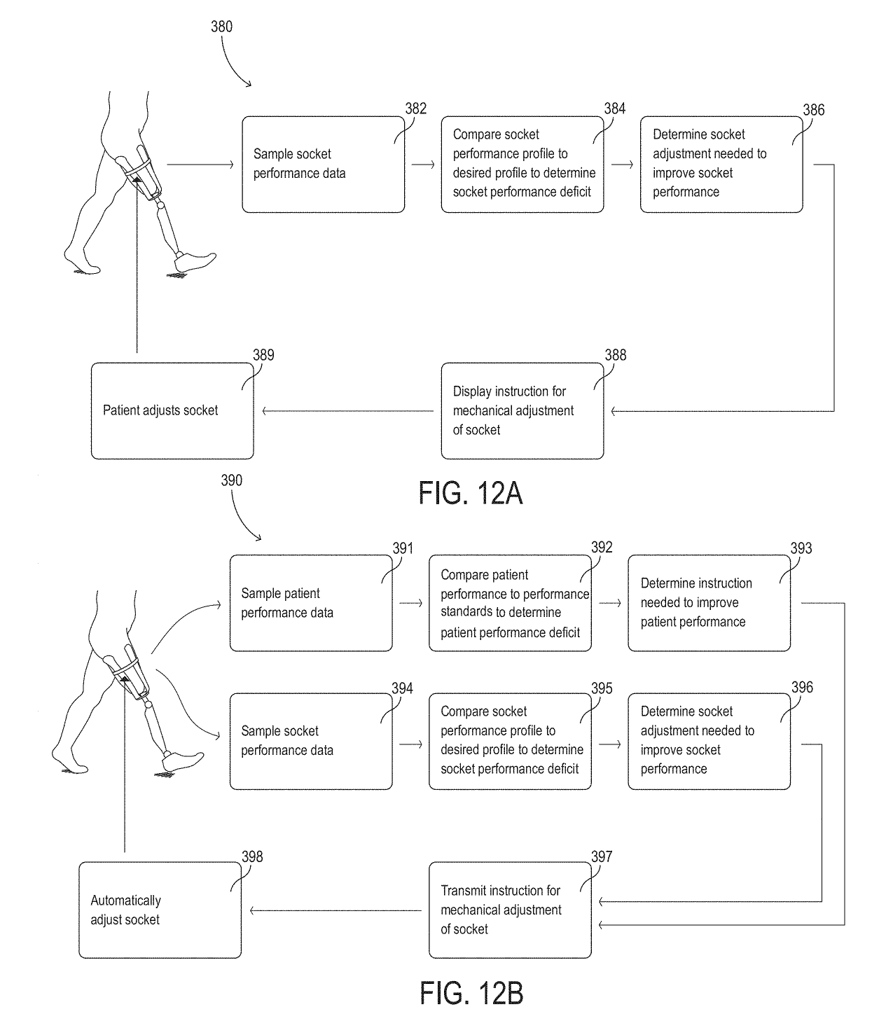

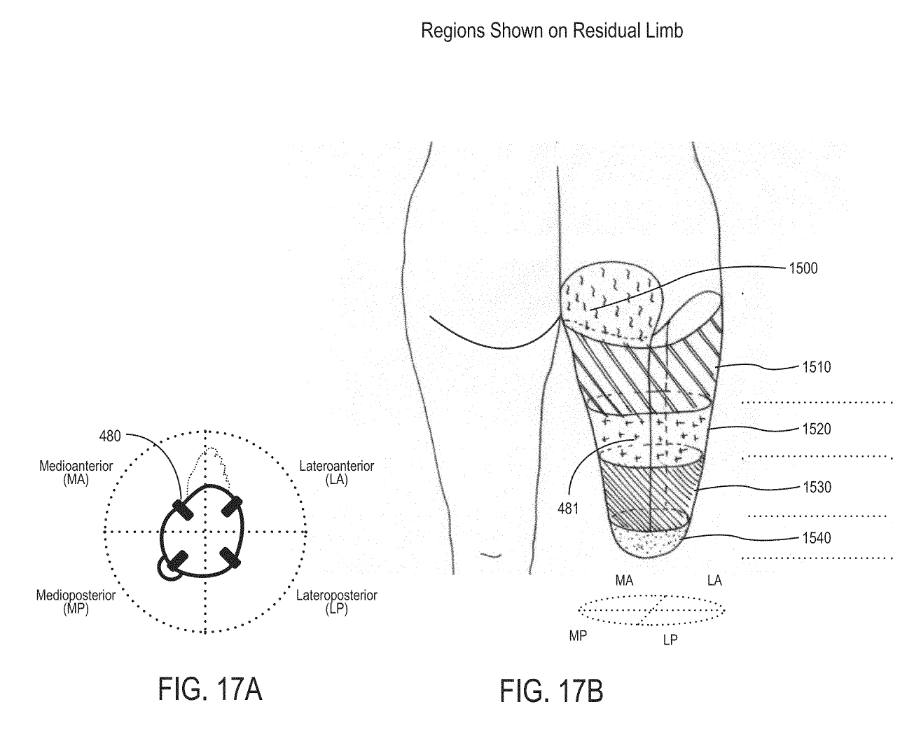

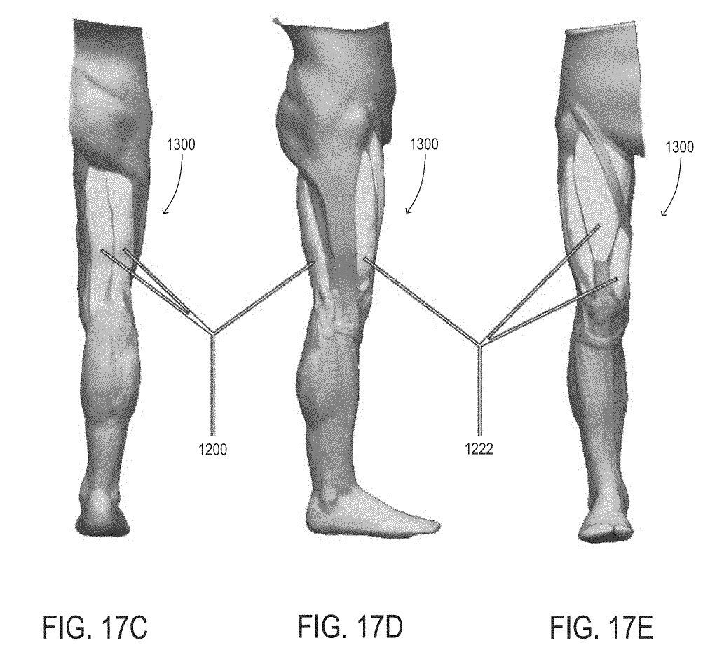

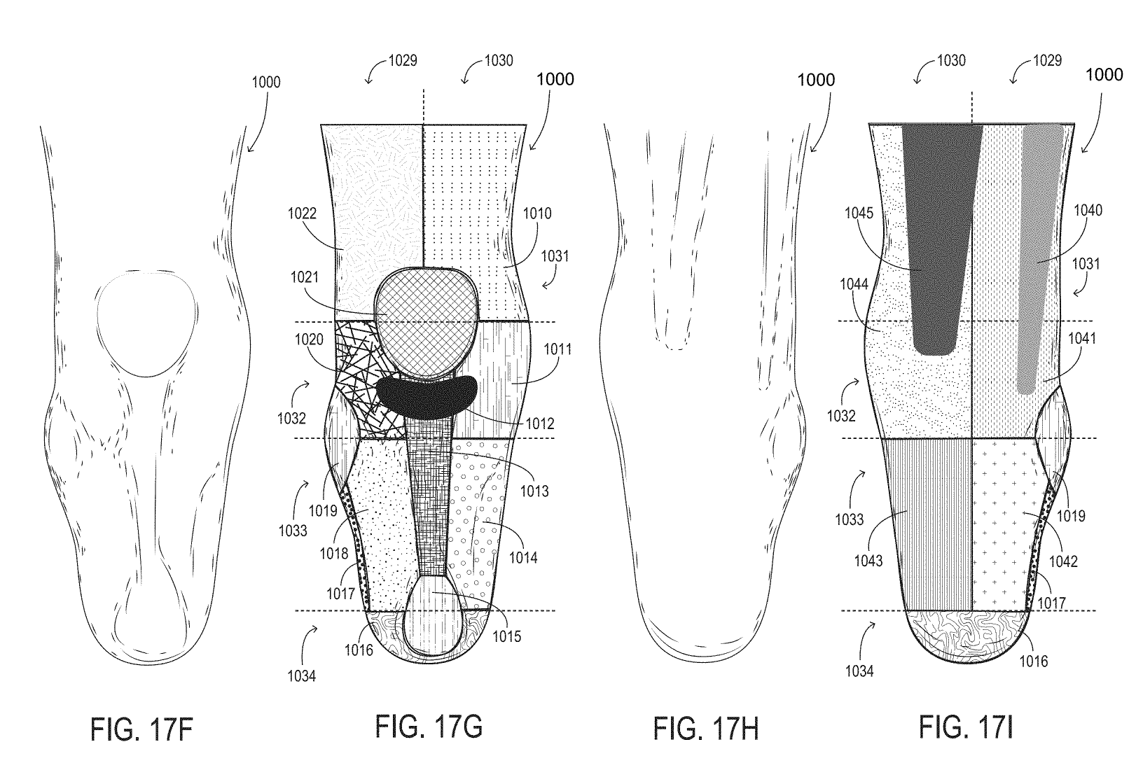

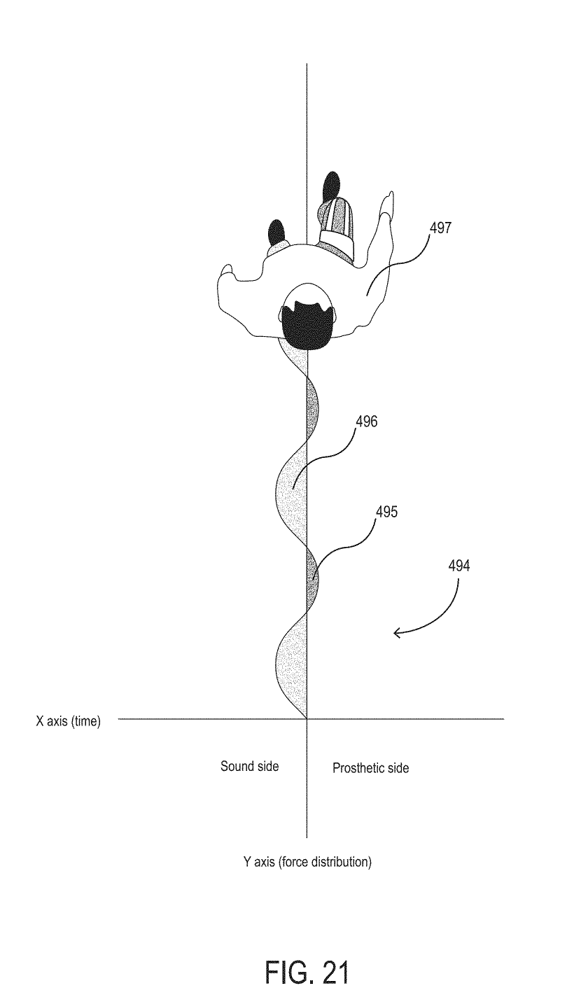

Embodiments of the technology include systems (FIGS. 9A-10 and FIGS. 13-14) and methods (FIGS. 11-12B) that rely on a sensor network in the socket to deliver force level information from throughout the socket, and which are used to profile the paths of force transfer from the body weight of the user through the distal base of the socket. FIG. 21 shows an example of how sensor-derived information can be processed into an analysis of user performance. FIGS. 17A-19 show examples of how sensor-derived information can reflect on a regional characterization of the socket state with regard to particular regions of the socket as well as particular regions of the user's residual limb. FIGS. 20A-20H show mobile application user-interface depictions of sensor-derived data that report on the physical state of the socket.

Force is generated by the body weight of the user even when the user is standing still, and as body weight is amplified by acceleration and impact associated with ambulation. A prosthetic socket performance profile or socket state refers to comprehensively processing sensor-derived data into a mapping of the distribution of forces as they flow from the body weight of the user through the socket from its proximal end to its distal end. A force profile, as used herein, may further include processed sensor data reporting on acceleration occurring within the socket, by region, and position of socket components in space and in relation to each other.

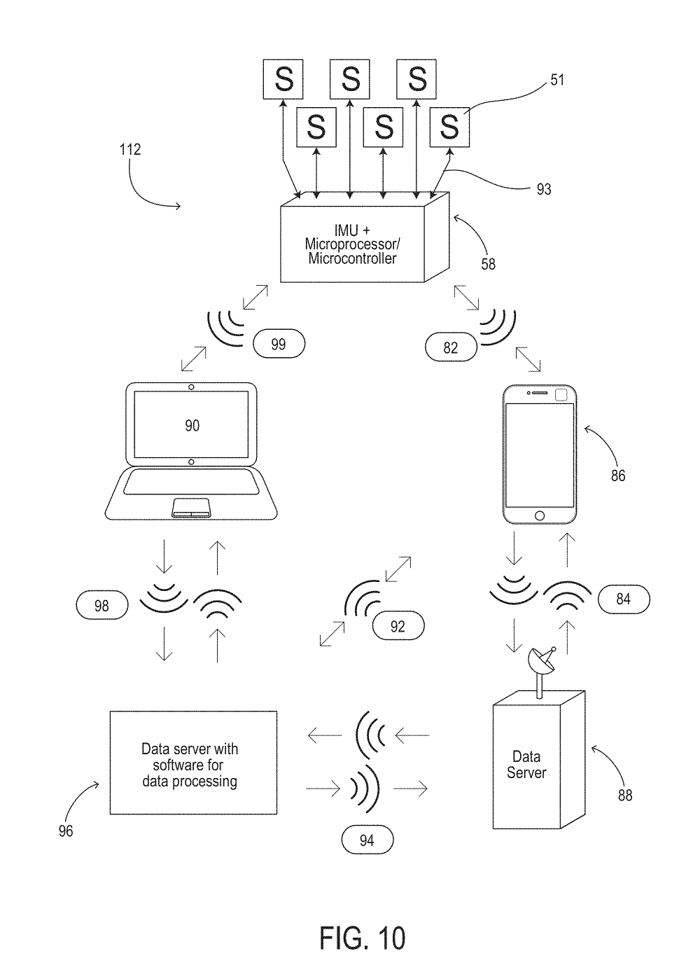

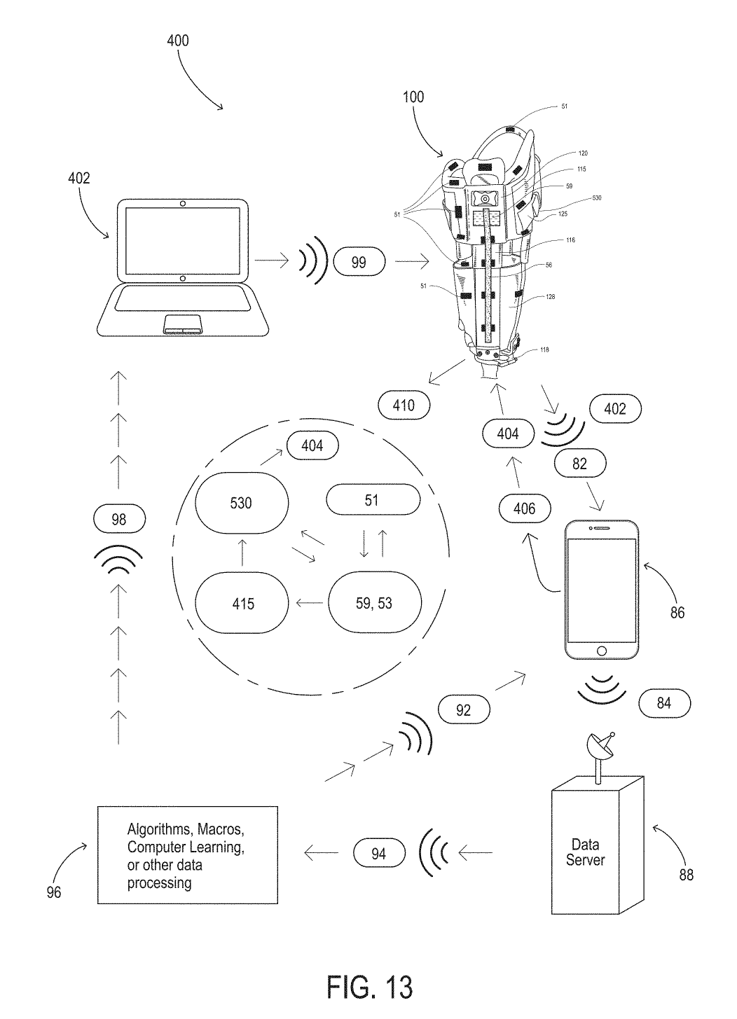

This profile information can be used in two basic ways, per various embodiments: (1) a strictly “informational” system (such as FIG. 10) in a format that can be displayed immediately, and which can also be accumulated and used in a retrospective manner, and (2) an actuatable and feedback loop system (such as FIG. 13) and manner in which profiled information is directed to motorized actuators that respond to bring the force path distribution profile toward a desired profile.

Informational system embodiments are useful as the basis for making manual adjustments of the prosthetic socket. Such embodiments may also provide alerts when data show a deviation from a predetermined desired profile of distribution of force through the prosthetic socket. In actuatable embodiments, building on an underlying informational system, processed sensor data can be applied to actuate adjustment features of the prosthetic socket that are activated when data report a deviation from a desired profile of distribution through the socket. The actuatable features, acting on instructions from system embodiments, respond by changing the configuration of the prosthetic socket in such a way as to drive the profile of force distribution toward a desired optimal profile. Force distribution profiles can be rendered as visually intuitive dashboard style diagrams, appropriate for a graphic user interface. In the actuatable aspect of the embodiments, the overall force profile is used as feedback to drive appropriate movement of motorized mechanical features of a prosthetic socket, such as, merely by way of example, a tension-based closure system, a hinge, or an adjustable height ischial seat.

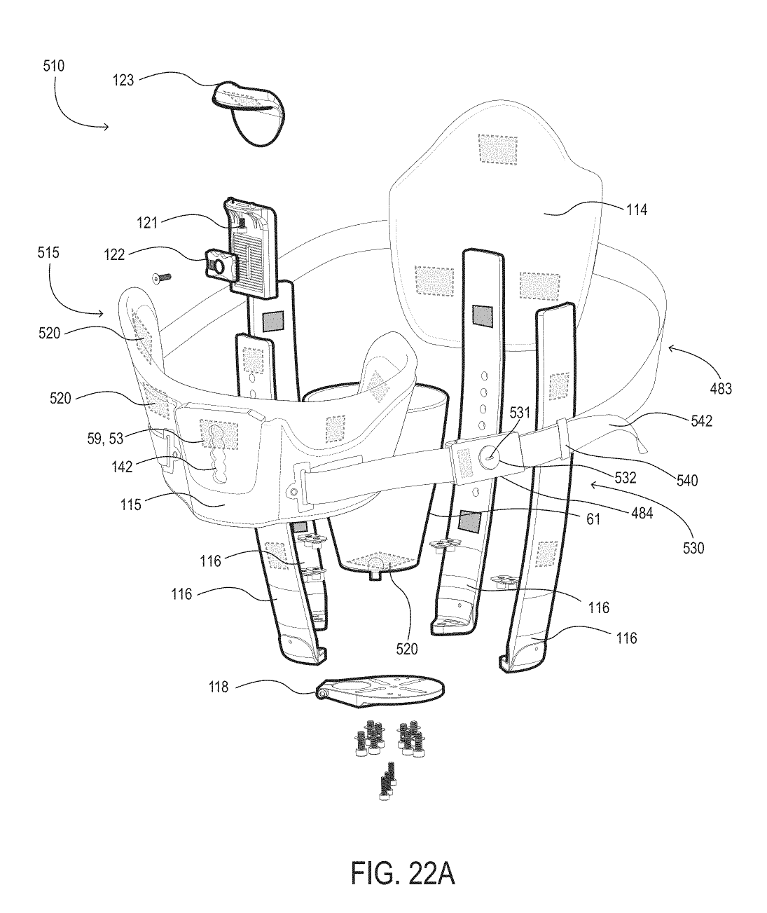

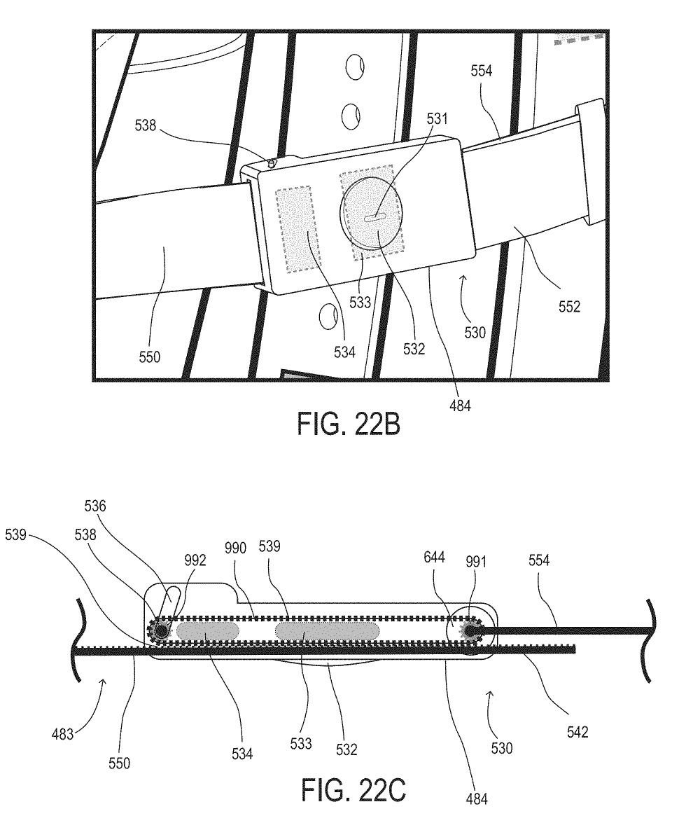

By way of one particular example of a force distribution issue, it is generally advantageous to distribute force away from the distal end of a residual limb, diverting force through the peripheral structures of the prosthetic socket. Profiling the force distribution paths within a prosthetic socket when worn by a patient will reveal when an inordinate amount of force is conveyed through the distal end. When the force profile of an empirically determined optimum is compared to a profile that shows too much force is conveyed through the distal limb, one or more circumferential tensioning mechanisms may be actuated by the system to distribute the force profile back toward a previously established optimal or desired profile. (FIGS. 22A-24E) illustrate an example of how show this condition and correction by way of dashboard style images.)

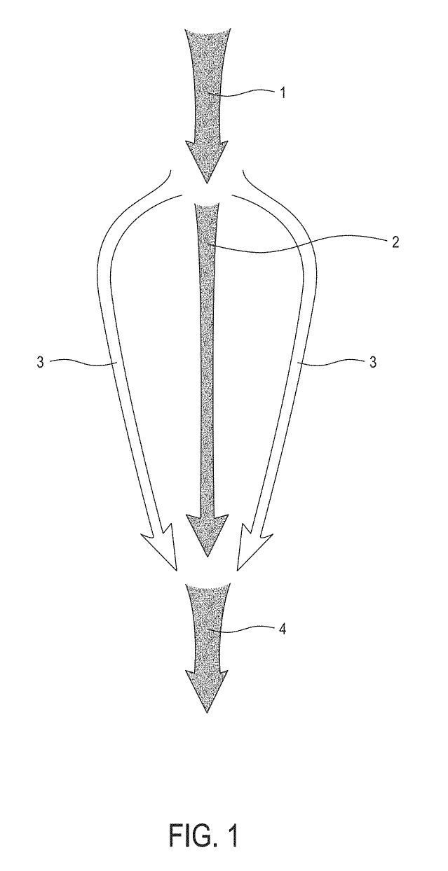

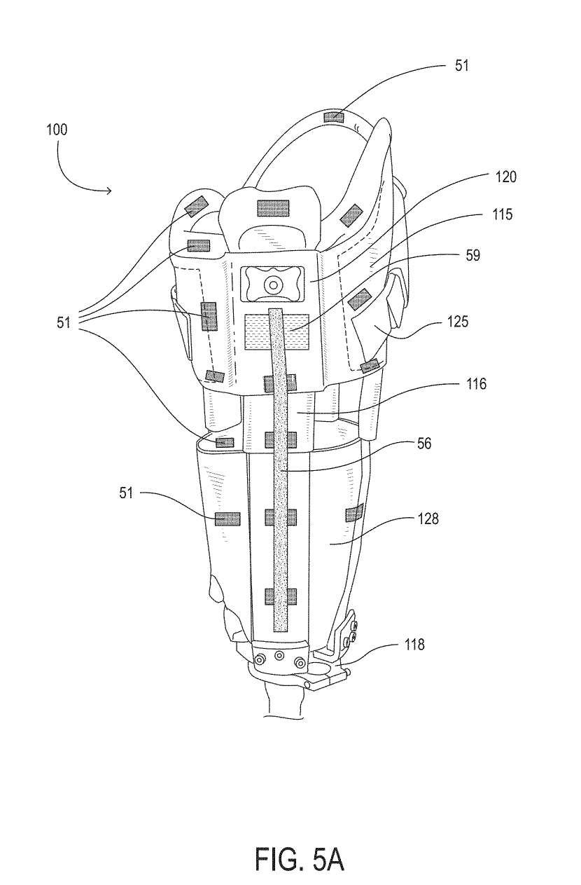

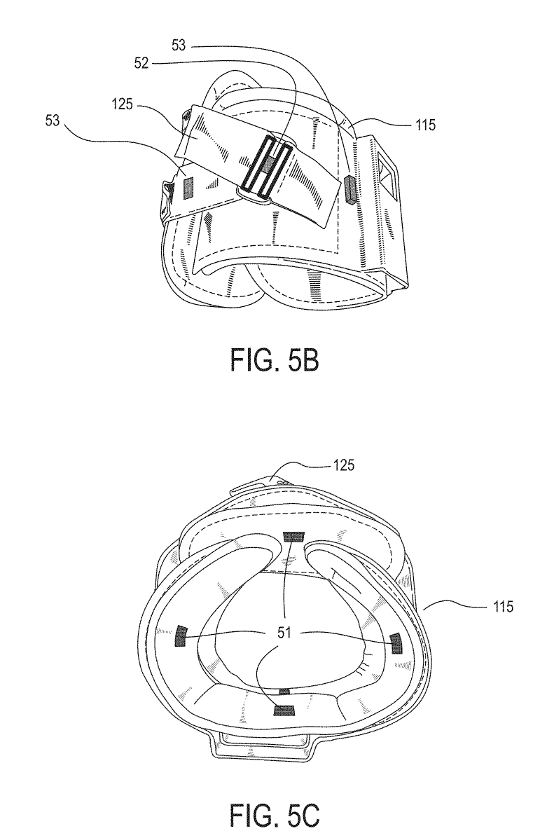

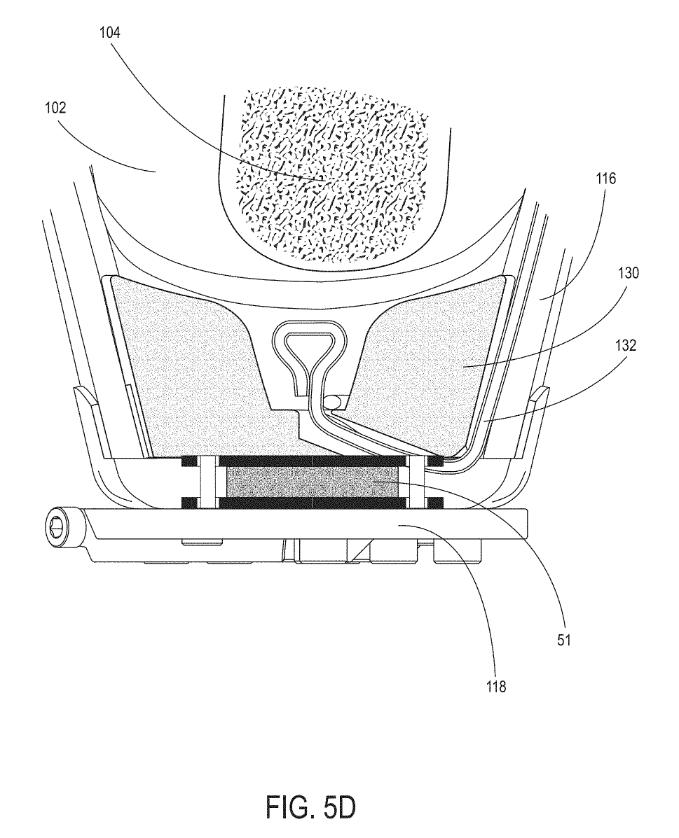

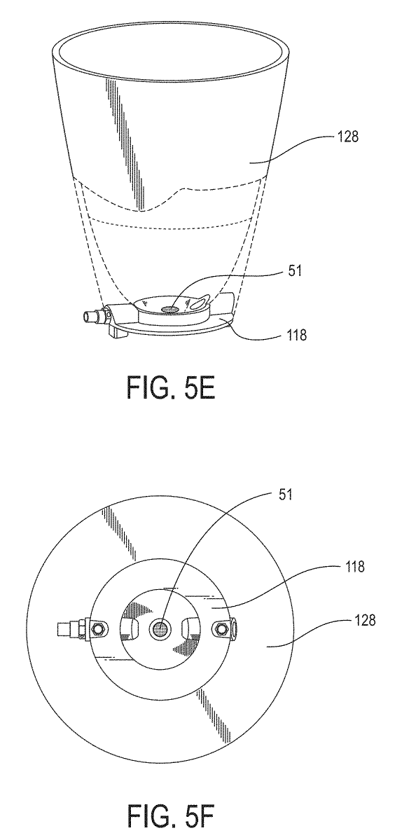

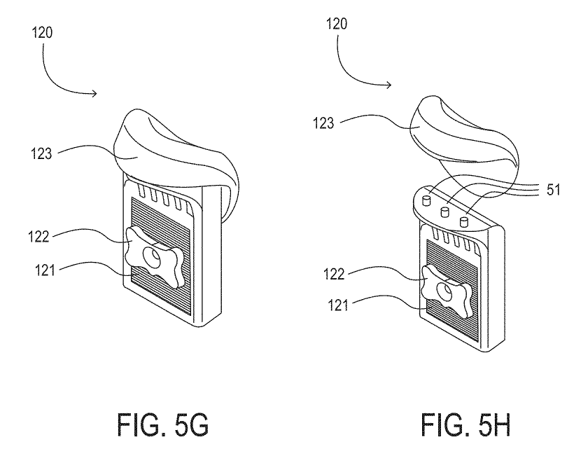

As shown in FIGS. 1-4, force is conveyed from a patient's body through a prosthetic socket by various routes that converge at the distal base of the socket and are conveyed distally into distal components as a single force. A network of sensors placed at various sites (FIGS. 5A-5H) in the socket may be used to capture force data, and by way of data processing, a profile of force distribution through the socket can be determined. Various embodiments of enabling systems and methods are described further below. In the descriptions that follow, the terms “patient” and “user” may sometimes be used interchangeably to describe any person who wears a limb prosthetic and thus a prosthetic socket. The term “user” may also be used to describe a family member, physician, prosthetist, other healthcare worker, socket manufacturing company or the like, who uses some part of a smart socket system or other device or system described herein, typically to help care for the patient. These terms—“patient” and “user”—should not be interpreted as limiting the scope of the invention.

Referring now to FIG. 1, a schematic representation is provided, showing the distribution of forces from the body weight and residual limb of a user into and through a prosthetic socket worn by the user. In FIG. 1, neither the residual limb nor the socket itself is shown. In this illustration, a proximal force 1 (in other words, the forces applied at the proximal end of the prosthetic socket by the patient's residual limb and body) is transmitted distally from the body weight of the user and separates ultimately into two paths. By way of a central path 2, force continues distally through the bone of the central limb and ultimately onto the distal base of the socket. By way of a peripheral path 3, force is dispersed into the socket structure and is conveyed distally, ultimately onto the distal socket base. Central and peripheral paths converge in a distal force 4 (or “convergent path 4”) at the distal base (in other words, the forces applied at the distal end of the prosthetic socket) and continue on to distal prosthetic components.

All proximal force 1 enters the prosthetic socket from the body weight of a user, positioned proximal to the socket. Proceeding distally, force distributes into central path 2 and peripheral path 3. Central path 2 represents the force conveyed through the residual limb (that which is not bled away into the peripheral path), and which is ultimately transferred through the distal end of the residual limb. Peripheral path 3 represents body weight driven force absorbed by a proximal component of a socket, such as a brim in the case of a transfemoral prosthetic socket (from the full circumference of the residual limb, but particularly from the ischial seat). Force from the brim is transferred distally through the longitudinal socket structures, such as struts. Force from the residual limb is also transferrable from the residual limb to the struts throughout their full length. Convergent path 4 represents the convergence of the peripheral path 2 and the central path 3 at the distal base of the prosthetic socket. The summed forces of the convergence path 4 are transferred distally from there to distal prosthetic components.

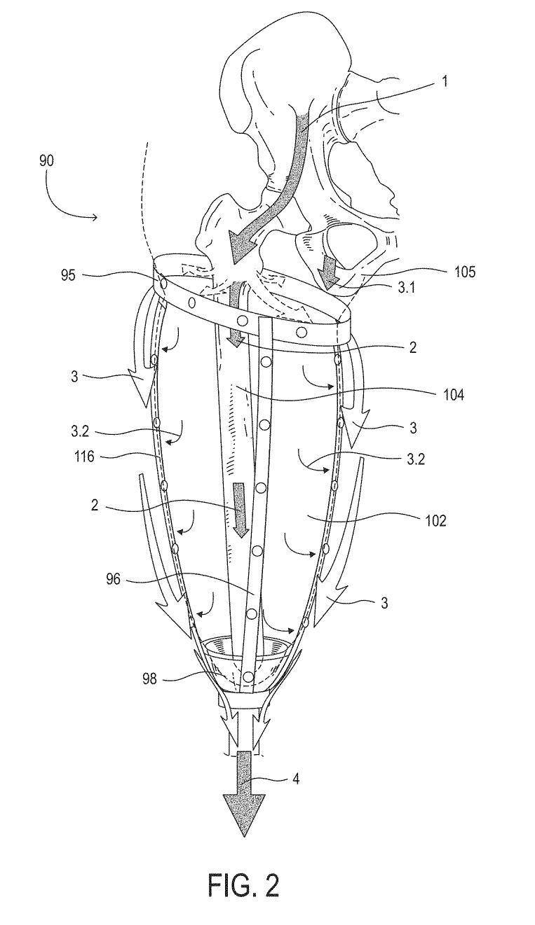

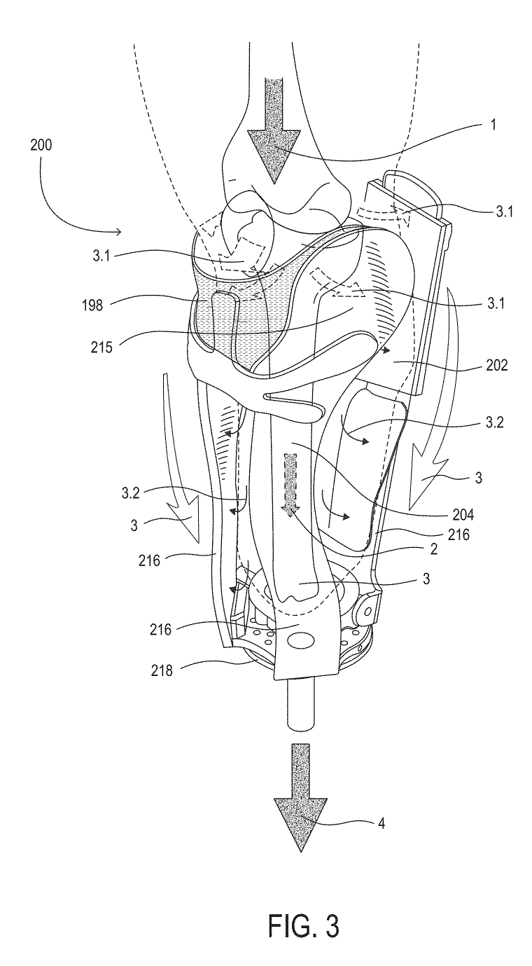

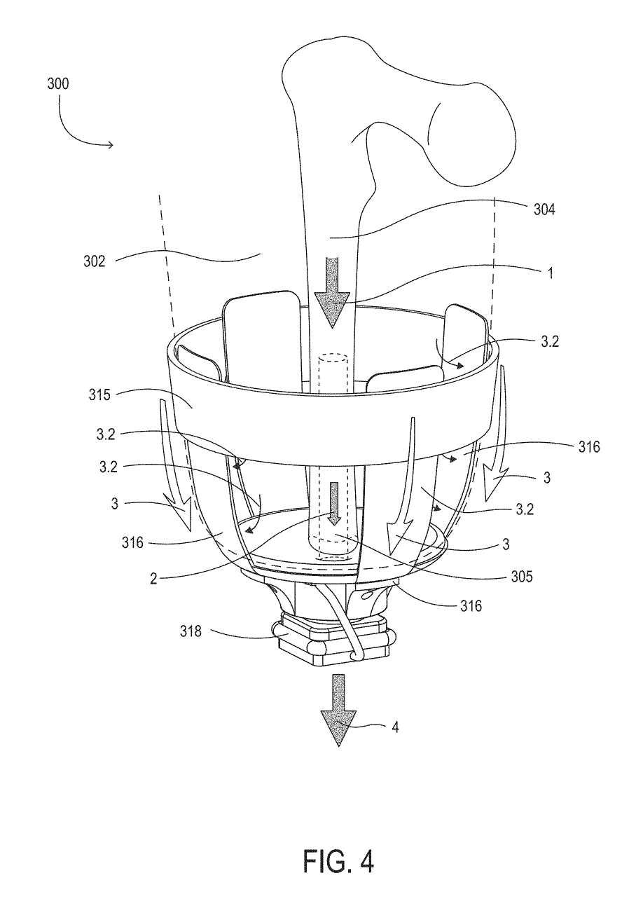

FIGS. 2-4 show this basic pattern of force distribution in greater detail, with variations on the common theme depicted in FIG. 1, and overlaid onto different prosthetic socket embodiments. FIG. 2 is a schematic representation of a transfemoral prosthetic socket 90, according to one embodiment. FIG. 3 is a more detailed view of a transtibial prosthetic socket 200, according to one embodiment. FIG. 4 illustrates an osseointegrated abutment support socket 300, according to another embodiment. The main purpose of these figures is to depict a common theme with regard to the distribution of body weight driven force through the respective sockets. The totality of body weight driven force as it enters or engages a prosthetic socket at its proximal end (proximal force 1) is equal to the distal force 4 as it exits the distal end of the prosthetic socket. Within the prosthetic socket, the totality of the proximal (or “entering”) force 1 diverges into the central path 2 through the central bone of the residual limb and the peripheral path 3 that is directed distally through the prosthetic socket structure, away from the central bone of the residual limb. Peripherally directed force arrives at socket structures either by (a) engagement 3.1 of bony structures of the residual limb against proximally situated structural features of the socket, or (b) engagement 3.2 of soft tissues of the residual limb against longitudinal structural features of the socket.

Each of these three exemplary prosthetic sockets has been previously described in detail. Examples of a modular transfemoral prosthetic socket are described in U.S. Pat. No. 8,978,224, entitled “Modular Prosthetic Sockets and Methods for Making Same”, and in U.S. Patent Application Pub. No. 2014/0277584, entitled “Modular prosthetic sockets and methods for making and using same,” now abandoned. Examples of a modular transtibial prosthetic socket are described in U.S. Provisional Patent Application Nos. 62/237,204, filed Oct. 5, 2015; 62/287,702, filed Jan. 27, 2016; and 63/305,477, filed on Mar. 8, 2016. Examples of a modular osseointegrated abutment support socket are described in U.S. Provisional Patent Application Nos. 62/197,427, filed Jul. 27, 2015; 62/267,820, filed Dec. 15, 2015; and 62/334,791, filed May 11, 2016. All of the above-listed patent applications are hereby incorporated in their entireties herein.

FIG. 2 shows an upper perspective view of a schematic rendering of an embodiment of a transfemoral prosthetic socket 90 hosting a patient's ischium 105 and residual limb (thigh) 102, including the residual femur 104. Proximal force 1 represents the entire force driven by the body weight and mass of the user though ischium 105 and residual limb 102. Proximal force 1, upon engaging prosthetic socket 90, diverges into two basic paths. Central path 2 continues down through the femur 104, ultimately impacting the distal base 98 of the socket 90. Force path 3.1 is directed toward a proximal brim 95 of the prosthetic socket, whereupon it is directed distally down longitudinal structures (typically struts) 96, as represented by peripheral force path 3. Some force, as represented by arrows 3.2, is directed proximally through residual limb 102, but it is diverted laterally onto longitudinal structures 96, thence joining distally directed peripheral force 3. Ultimately, the peripheral path 3 represents that portion of proximal force 1 from the user that is directed peripherally through socket 90, and away from distal base 98. Central force path 2 represents that portion of proximal force 1 from the user that remains directed through the distal end of the femur 104 and on to distal base 98. At the distal portion of socket 90, the central force path 2 and the peripheral force paths 3 converge and continue distally as distal force path 4 (which is equal in magnitude to force 1).

In one aspect, a function of prosthetic socket 90 is to divert the proximal force 1 peripherally as peripheral forces 3, thereby minimizing the magnitude of the central force 2 through the distal end of the femur 104, which is not physically suited to bear significant force, and which is sensitive to such force. As described herein, one of the functions of smart socket systems is to quantify the level of force driven through the distal end of femur 104 and provide instructions or make automatic adjustments within prosthetic socket 90 to direct force peripherally, away from the distal end of femur 104. This is true of the following two embodiments illustrated in FIGS. 3 and 4 as well.

FIG. 3 shows an upper perspective view of a transtibial prosthetic socket 200 according to another embodiment. The socket 200 is shown hosting a patient's residual limb 202, including a residual tibia 204. The proximal force path 1 represents the entirety of force driven by the body weight and mass of the user through the patella and condyles associated with the knee. The proximal force path 1, upon engaging prosthetic socket 200, diverges into two basic paths. The central force path 2 continues down through tibia 204 (and fibula, which is not shown), ultimately impacting a distal base 218 of the socket 200. Force path 3.1 is directed toward proximal structures 215 of the prosthetic socket 200, whereupon force is directed distally down longitudinal structures (typically struts) 216 of the socket 200, as represented by peripheral force path 3. Some force, as represented by arrows 3.2, is directed proximally through the residual limb 202, but is diverted laterally onto longitudinal structures 216, thence joining distally directed peripheral force path 3. Ultimately, the peripheral force path 3 represents that portion of the proximal force path 1 from the user that is directed peripherally through socket 200 and away from distal base 218. The central force path 2 represents that portion of the proximal force path 1 from the user that remains directed through the distal end of tibia 204 and on to distal base 218. At the distal portion of socket 200, the central force path 2 and the peripheral force paths 3 converge and continue distally as distal force path 4 (which is equal in magnitude to force 1).

FIG. 4 shows an upper perspective view of an osseointegrated abutment support socket 300, according to another embodiment. The socket 300 is shown hosting a patient's residual limb 302, including the residual femur 304. The proximal force path 1 represents the force driven by the body weight and mass of the user though the patella and condyles of the knee. The proximal force path 1, upon engaging osseointegrated abutment support socket 300 diverges into two basic paths. The central force path 2 continues down through femur 304 and thence into an abutment 305 of the socket 300, ultimately impacting a distal base 318 of the socket 300. Force path 3.1 is directed toward a proximal structure (a brim) 315 of the prosthetic socket 300, whereupon force is directed distally down longitudinal structures (typically struts) 316 of the socket 300, as represented by peripheral force path 3. Some force, as represented by arrows 3.2, is directed proximally through the residual limb 302 but is diverted laterally onto longitudinal structures 316, thence joining the peripheral force path 3. Ultimately, the peripheral force path 3 represents that portion of the proximal force path 1 from the user that is directed peripherally through socket 300 and away from distal base 318. The central force path 2 represents that portion of the proximal force path 1 from the user that remains directed through the distal end of femur 304 and on to distal base 318. At the distal portion of socket 300, the central force path 2 and the peripheral force paths 3 converge and continue distally as distal force path 4 (which is equal in magnitude to force 1).

Referring now to FIGS. 5A-5H, one embodiment of a modular transfemoral “smart” prosthetic socket 100, which may act as a component of a smart prosthetic socket system, is illustrated. The socket 100 itself may be the same as or similar to any of the sockets disclosed in the patents and patent applications incorporated herein by reference, for example. This embodiment of the smart socket 100 includes sensors of various types positioned at suitable and informative positions within the smart prosthetic socket 100. Some sensors, such as motion sensors, may be positioned in any number of locations and provide informative data. Other sensors, such as force resistive sensors, may be most suitably positioned at interfacing or junctional sites, such as where separate structures engage, and across which forces are transmitted. Accordingly, in the examples shown, sensors are shown at sites that are suitable for the particulars of the socket structure. The smart socket and sensor technology described herein is not limited to use in the specific prosthetic sockets shown in this application, but rather to a full range of prosthetic sockets, as each type of prosthetic socket will have appropriate sites for reporting sensed data, and the data will be applicable to clinical uses as described herein.

FIG. 5A is side view of the transfemoral prosthetic socket 100, which includes a distal base 118, longitudinal struts 116, one or more proximal brims 115, and a distal flexible inner liner or distal cup128 positioned within the distal portion of the cavity formed by the longitudinal struts 116. Prosthetic socket 100 further includes force sensors 51 and a flex sensor 56 positioned at various sites, as well as a combination microcontroller/data transmission unit/battery 59 attached to the prosthetic socket 100.

FIGS. 5B and 5C are perspective and bottom views, respectively, of the proximal brim 115 (or simply called “proximal portion” or “proximal components”) of the transfemoral prosthetic socket 100. When in use, the brim 115 is coupled with the proximal ends of some or typically all of the longitudinal struts 116, stabilizing them with an appropriate amount of tension, as adjusted by way of a tensioning or closure belt 125. The brim 115 may fit over, around, inside of and/or between the struts 116. Two types of sensors are shown in FIG. 5B—a motion sensor or accelerometer 53 and a belt position and tension sensor or strain gauge 52. In one embodiment, the motion sensor 53 may located in a microprocessor/sensing unit on the exterior of the socket 100. The motion sensor 53 may be a 9-axis motion detection unit that includes a 3-axis accelerometer, a 3-axis gyroscope, and a 3-axis magnetometer. The belt position and tension sensor unit 52 may be positioned within a buckle that connects the two-part tensioning belt 125 on the exterior of the brim 115. The tension sensor 52 in this embodiment is a strain gauge, and the position sensor is a Hall type sensor that also includes a reference magnet on the tensioning belt 125 at a distance from the buckle.

FIG. 5C is a bottom view of the brim 115, looking into the interior of the brim 115, which includes an inner surface that in some embodiments directly contacts the residual limb and in other embodiments contacts a liner worn by the user. Brim 115 may be tensioned around the residual limb, thereby imparting force directed inwardly against the limb. Thus, this interfacing space is suitable for force resistive sensors 51 that can measure such force. Force resistive sensors 51 may be distributed circumferentially at various sites on the interior aspect of brim 115 and networked together. Each sensor 51 may sense and provide data corresponding to the local pressure, and the circumferential network as a whole can provide data that compares the relative distribution of pressure around the limb, within the brim.

FIG. 5D is a side view of a distal portion of the transfemoral prosthetic socket 100, including distal base 118, longitudinal struts 116, a distal pad 130 cushioning the distal end of the residual limb 102 and its residual femur 104, and a lanyard strap 132 for a lanyard suspension system. A force sensor 51 is positioned between the distal pad 130 and the underlying distal base 118. Force sensor 51, in this position, is capable of sensing the full amount of body weight driven force directed through the distal end of residual femur 104.

FIGS. 5E and 5F are a partially transparent side view (FIG. 5E) and a bottom view (FIG. 5F) of a distal cup 128 (or “distal flexible inner liner”) of the transfemoral prosthetic socket 100, illustrating the placement of a force sensor 51 at a distal end of the cup 128. Embedded within the distal end of the flexible distal cup is a portion of the distal base 118, which may be referred to as a distal base plate or support plate and which includes plumbing to allow the controllable flow of liquid or air that accumulates between the distal cup 128 and the residual limb. When the prosthetic socket 100 is worn, the distal cup 128 resides within the prosthetic socket 100, positioned on top of the distal base 118 of the socket 100, and the cup 128 hosts the distal end of the residual limb. At least some of the body weight of the wearer is thus transmitted from the residual limb to the distal base 118 of the socket across interfacing space in the distal center of the distal cup 128. Accordingly, this interface is a suitable site for capturing transmitted force by way of force resistive sensor.

FIGS. 5G and 5H are perspective and exploded views, respectively, of an adjustable height ischial seat 120 of the transfemoral prosthetic socket 100. The ischial seat 120 includes an ischial seat pad 123, an ischial seat base 121, an ischial seat locking element 122, and in this embodiment multiple sensors 51. The ischial seat base 121 is configured to be disposed at the proximal end of a medial strut of a transfemoral prosthetic socket 120. The exploded view of FIG. 5H shows that force sensors 51 may be positioned between the ischial seat base 121 and the ischial seat pad 123.

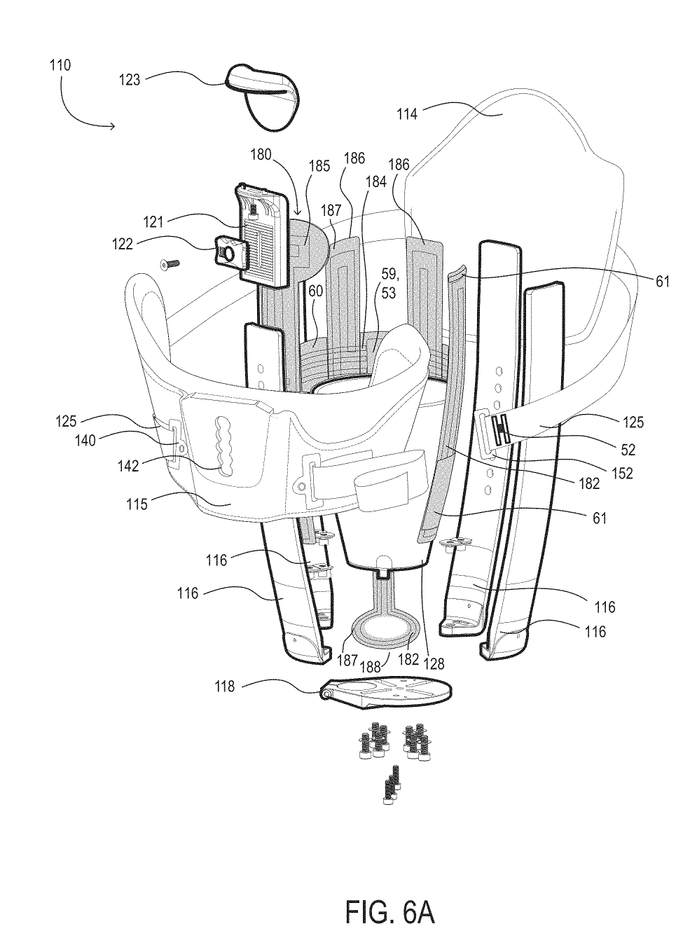

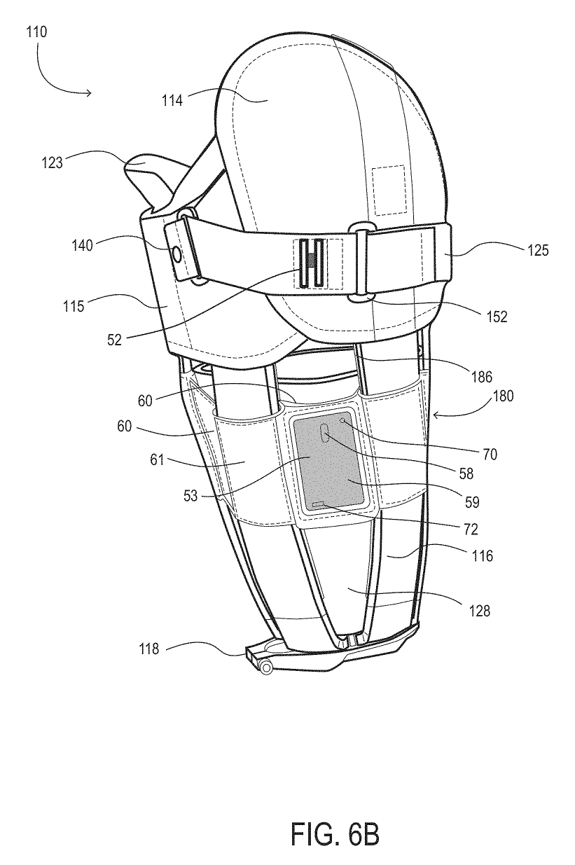

With reference now to FIGS. 6A and 6B, another embodiment of a modular transfemoral sensor-enabled prosthetic socket 110 may include a distal base 118, longitudinal struts 116, an ischial seat pad 123, an ischial seat base 121, an ischial seat locking element 122, a proximal brim 115, a lateral brim component 114, a distal cup128, a swiveling chafe 140, a longitudinal adjustment mechanism 142, a tension belt 125, and a D-loop 152. This figure also shows a modular addition of a data collection and transmission system 180 with longitudinal sensing segments 186, including fabric or plastic substrate layer(s) 187 and including integrated custom pressure sensors 182 (or “pressure sensor material layup”), which, in this embodiment, are custom shaped and constructed from two conductive layers, such as copper on either side of Velostat™, which is made of polymeric foil or polyolefines impregnated with carbon black. The custom pressure sensors 182 may be connected to the microcontroller/data transmission unit/battery 59 via flexible and stretchable conductive fabric 184 and flexible and stretchable fabric sections 60 between longitudinal sensor segments 186. Flexible and stretchable fabric pocket sections 61 are positioned on the outer facing aspect of the data collection and transmission system 180, in order to easily mate or configure with the longitudinal struts 116. The distal pressure sensor segment 188 also includes the custom pressure sensors 182 and fabric or plastic substrate layer(s) 187, but this section is configured for placement between the distal cup 128 and the distal base 118, so that it can sense the amount of pressure applied through the distal aspect of the socket 110. In this embodiment, a proximal section 185 of one of the longitudinal sensor segments 186 is configured to couple with the ischial seat base 121, the ischial seat pad 123 or other ischial weight bearing component(s) of the socket 110, for example by fitting into a pocket on the ischial seat base 121 or the ischial seat pad 123.

The microcontroller/data transmission unit/battery 59 (FIG. 6B) may include an integrated motion sensor or accelerometer. As described previously, the motion sensor may be situated within a microprocessor and sensing unit and may include a 9-axis motion detection unit that includes a 3-axis accelerometer, a 3-axis gyroscope, and a 3-axis magnetometer. The tension sensor or strain gauge 52 may also be situated on the tension belt 125, and in some embodiments it may be the only sensor on the prosthetic socket 110 that is not contained within the data collection and transmission system 180. Strain gauge 52 may use a wireless communication with microcontroller/data transmission unit/battery 59.

FIG. 6B is a perspective view of the transfemoral prosthetic socket 110. This assembled perspective shows how the flexible and stretchable fabric sections 60 can stretch to accommodate differently shaped residual limbs having different distances between longitudinal struts 116 and longitudinal sensor segments 186. FIG. 6B also shows how the flexible and stretchable fabric pocket sections 61 fit around the longitudinal struts 116 of the modular prosthetic socket 110. This figure also shows how the swiveling chafe 140, tension belt 125, and D-loop 152 can be configured on the assembled socket 110, such that they provide an adjustment mechanism for the user to loosen or tighten the prosthetic socket 110. FIG. 6B also shows the outer aspect of the microcontroller/data transmission unit/battery 59, which may include a power button 58, an on/off indication LED light 70, and a charging port 72.

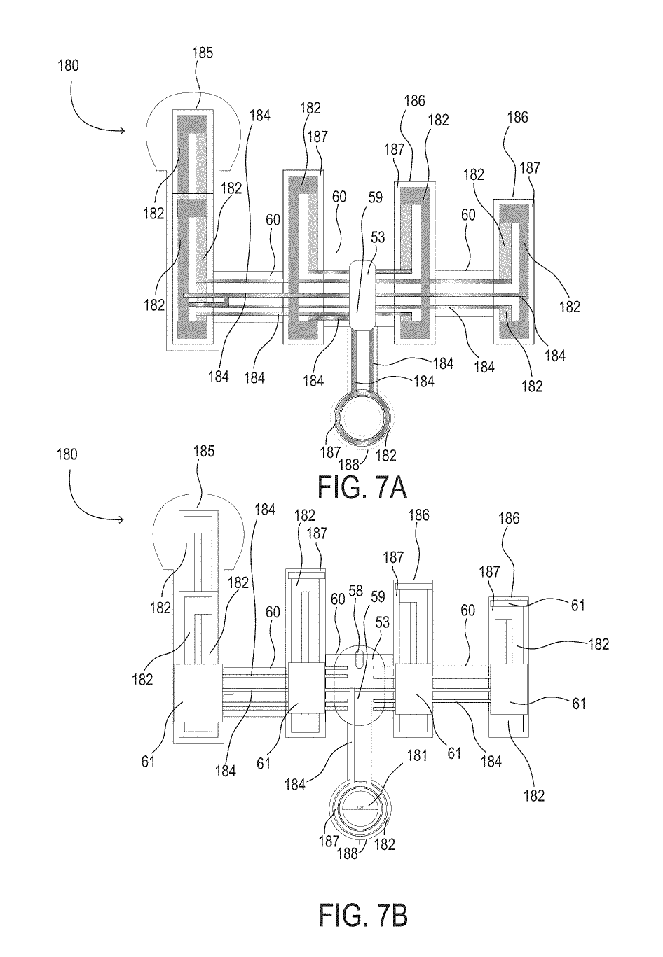

Referring now to FIGS. 7A and 7B, top views of the modular data collection and transmission system 180 of FIGS. 6A and 6B are shown in a flattened configuration. The modular data collection and transmission system 180 shown in FIGS. 7A and 7B include all of the parts and features described above, so those descriptions will not be repeated. FIG. 7A does not show the elastic belt loops used to couple with the longitudinal struts 116 of the socket 110, in order to show the entire path of the sensors and connection to the microcontroller/data transmission unit/battery 59. FIG. 7B does show the elastic belt loops. FIG. 7B also provides a scale measurement 181, which shows the dimension of this portion of the modular data collection and transmission system 180 and a scale for the size of the entire system 180. For example, in this embodiment the scale measurement reads “1.6 in” (i.e., 1.6 inches), although it may have a different measurement in other embodiments. The modular data collection and transmission system 180 may be offered in one size or multiple sizes. The elastic portions of connection sections 60 and 184 are intended to allow for accommodation of many different socket shapes with minimal inventory.

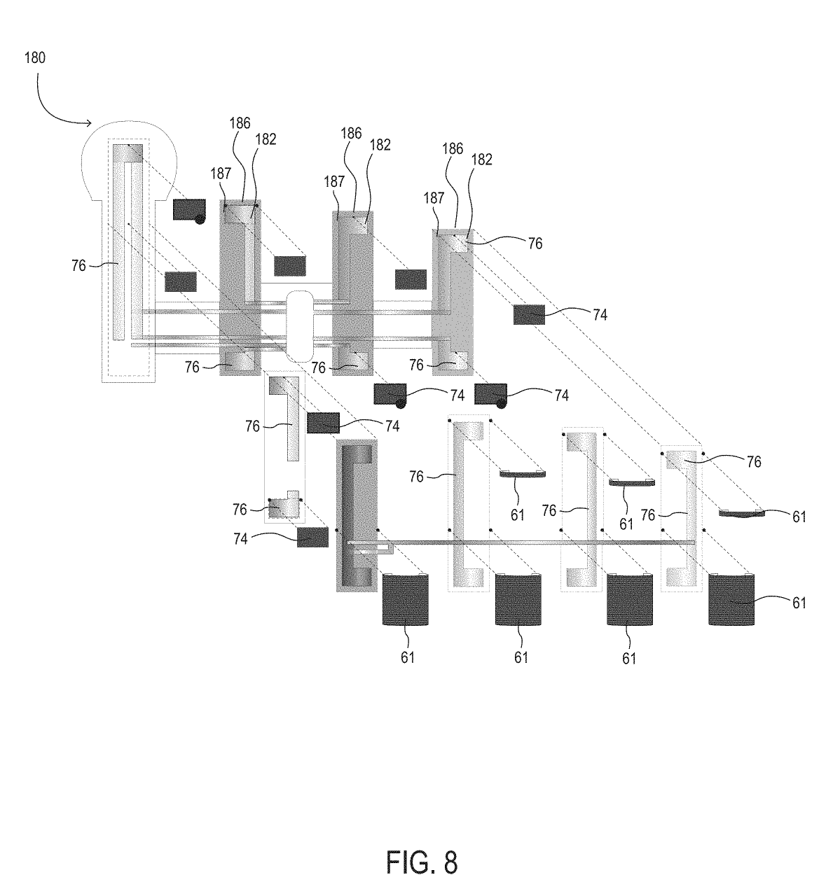

FIG. 8 is an exploded view of the modular data collection and transmission system 180 of FIGS. 6A-7B. This view shows the various parts described above and also shows that the longitudinal sensing segments 186 are made up of several layers of material textile or plastic, to make the fabric or plastic substrate layers 187 to hold integrated custom pressure sensors material layup 182 which, in this embodiment, is constructed of two conductive layers 76 (copper) and Velostat™ 74 between. This figure also shows how the flexible and stretchable fabric pocket sections 61 are constructed and oriented, relative to the other parts.

With reference now to FIG. 9A, a flow diagram illustrates a smart socket system and method for monitoring a prosthetic socket 110 or socket state, in which regionally specific socket data and other data are transferred (acquired, displayed, or transferred back and forth) wirelessly between the prosthetic socket 110 and one or more devices. In general, smart socket systems and methods include a smart prosthetic socket and at least one additional device or computer application on a device, with which the smart prosthetic socket communicates. The smart socket includes at least one sensor for sensing any of the parameters discussed above, related to the state of the socket, the state of the patient and/or the interaction of the patient with the socket. The additional device may be a computer application on a smart phone, tablet or other computing device, a processor, a server, a mobile computing device itself, such as a smart phone or tablet, a laptop or desktop computer, and/or any other such device or combination of devices. The smart socket may often communicate wirelessly with the additional device(s), but in some embodiments there may also be a wired connection option. For example, it may be possible in some embodiments to connect a smart phone to a smart prosthetic socket via a wired connection to download data. The various users of the smart socket system and method may be the patient, a prosthetist, a physician, another healthcare provider, a researcher, friends and family of the patient, and/or any other suitable users. Therefore, the examples shown in FIGS. 9A and 9B are provided for illustrative purposes only and are not intended to be limiting.

Referring again to FIG. 9A, in this embodiment of the smart socket system and method, data may be transferred 82 between the smart prosthetic socket 110 and a smart phone 86, data may be transferred 84 between the smart phone 86 and one or more servers 88, and/or data may be transferred 80 between the user and smart phone 86. A smart phone application may be downloaded onto the smart phone 86, to facilitate the interaction between the prosthetic socket 110 and the smart phone 86. The application, in some embodiments, may use the smart phone's camera 85, GPS unit 87 and/or user interface on a display screen 89 to facilitate information transfer between the user, the smart phone 86 and the socket 110. The display screen 89 may be used to display data, prompts and/or notifications, for example, and may also allow the user to input information into the application.

FIG. 9B is a flow diagram similar to that of FIG. 9A, with the addition of data being viewed 83 by an authorized viewer 91 via the smart phone 86 and/or a computer 90. Data may be transferred from the smart phone 86 to the computer 90, for example, and/or data may be transferred from the server 88 to the computer 90. The authorized viewers 91 may be healthcare workers, researchers, family members and/or the like, who are able to view 83 data or input data relating to use of the prosthesis, gait, and/or other health related data. Of course, the user/patient may also view data via the cell phone 86 and/or the computer 90.

Referring now to FIG. 10, a system diagram of another embodiment of a smart socket system 112 is provided. In this embodiment, the smart socket system 112 includes RFID communication, Bluetooth, or other form of data transfer 93 between sensors 51 within a sensor array and a inertial measurement unit (IMU)/microprocessor/microcontroller 58, which may be located on a prosthetic socket (not shown) and in some embodiments combined with a data transmitter and/or a batter. The IMU/microprocessor/microcontroller 58 may communicate wirelessly with a smart phone 86 via a smart phone software application, and the smart phone 86 may communicate wirelessly 84 with a remote server 88. The remote server 88 may communicate 94 with software for data processing 96 (within the same server or at a separate server). Either the data server 88 or the software 96 may communicate with an additional computing device 90, such as a laptop computer, and the computing device 90 may communicate 99 with the IMU/microprocessor/microcontroller 58. In some embodiments, the data may also be transferred 92 between the smart phone 86 and a remote server with software for data processing 96. As should be evident from FIG. 10, any combination of computing devices, data processing devices and communications between them is possible, according to various embodiments.

Referring now to FIG. 11, in one embodiment, a smart prosthetic socket data management system 340 may include a mobile application interface 342, a computer application 344 and a smart prosthetic socket sensor interface 368. The sensor interface 368 (or “sensor module”) includes or collects sensor data from any sensors on a prosthetic socket, such as but not limited to skin moisture data 370, heart rate data 372, pressure sensor data 374, accelerometer/IMU data 376 and/or belt tension sensor data 378. The computer application 344 may include any of a number of monitors and modules, such as but not limited to a skin moisture monitor 346, a heart rate monitor 348, a limb and socket analysis module 350 and/or a movement analysis module 360. In this embodiment, the limb and socket analysis module 350 includes a socket suspension measurement 352, a limb volume measurement 354, a socket tension measurement 356 and a socket pressure monitor 358, although different measurements and combinations are possible in alternative embodiments. In this embodiment, the movement analysis module 360 includes fall detection 362, gait analysis 364 and movement tracking 366, although other measurements and combinations are possible in alternative embodiments. According to various embodiments, the mobile application interface 342, the computer application 344 and the smart prosthetic socket sensor interface 368 may be located on one device or on multiple devices. The mobile application interface 342 and the computer application 344 may transmit and receive data back and forth with one another, and the computer application 344 and the sensor interface 368 may transmit and receive data back and forth with one another.

FIGS. 12A and 12B are method flow diagrams illustrating two embodiments of a method for using data acquired from a network of sensors distributed within a prosthetic socket. The data may be directed toward providing information related both to prosthetic socket performance and patient performance.

Referring to FIG. 12A, in one embodiment, a smart prosthetic data analysis and adjustment method 380 may initially involve sampling prosthetic socket performance data 382, which may include any data related to the performance of the socket, examples of which are described above. Next, a processor in the smart prosthetic socket system compares a socket performance profile generated from the socket performance data to a desired profile 384, to determine whether there is a socket performance deficit, and if so what the deficit entails. An intermediate step may be for the system to generate the socket performance profile from the socket performance data, although in other embodiments the data may simply be compared to the desired profile, without generating a performance profile for the socket. Next, the processor, or a different processor in the smart socket system, determines one or more socket adjustments needed to improve the socket performance 386, based on the comparison of the performance profile to the desired profile. The system then displays instructions for making one or more mechanical adjustments of the socket to remedy the deficit 388. This display may occur on a smart phone, tablet, laptop computer, any other computing device, or in some embodiments even on a display screen on the smart prosthetic socket itself. Finally, the patient (or physician, prosthetist or other helper) adjusts the socket according to the instructions 389.

Referring now to FIG. 12B, in an alternative embodiment, a smart prosthetic data analysis and adjustment method 390 may initially involve two data gathering and processing paths. One path involves the same steps as described in relation to FIG. 12A—sampling socket performance data 394, comparing a socket performance profile to a desired profile to determine socket performance deficit 395 and determining socket adjustments needed to improve socket performance 396. The other path involves sampling patient performance data 391, comparing patient performance to one or more performance standards to determine if there is a patient performance deficit 392 and determining instructions for improving patient performance 393. Patient performance data may include any physiological and/or biometric data reflective of the status of the residual limb or of the patient as a whole. In this embodiment, any instructions to improve patient performance 393 and adjustments to improve socket performance 396 are transmitted to the smart prosthetic socket 397. They may also be provided to the patient and/or other user(s), via any computing device. The generated instructions and/or adjustments are then used to automatically adjust the smart prosthetic socket 398, for example by controlling a motorized mechanical feature, automated inflation/deflation system, and/or other automated adjustment mechanism of the prosthetic socket. Information relating to prosthetic socket performance, adjustments made and/or patient performance may also be used in an informational manner, for example for research, machine learning or the like.

In some cases, a particular patient may have past data regarding patient performance and/or socket performance, and this past data can be used, for example, in generating desired patient and socket profiles. In other cases, a patient might not have any previous use data or that data might not be available. In those cases, it may be possible to use data from a broad group of users to create patient performance standards and/or socket profiles. In some cases, through previous use and/or testing of a prosthetic socket on a patient, a full range of available mechanical adjustments may be recorded, and optimal prosthetic socket performance profiles may be created for respective activities. Such activities may include, for example, lying down, sitting, standing, walking slowly and walking quickly. Each of these activities can be captured and recognized by profile signatures derived from sensor data (including input from force, acceleration, and position sensors). An overall preferred profile of each of these types of activities becomes the “individual standard,” against which any set of instant observations is compared for the patient. This individual standard can be compared and contrasted to general performance standards derived across different user profiles in order to extrapolate relevant clinical learning that may inform refinement of either individual standards or general performance standards.

Referring now to FIG. 13, another embodiment of a smart prosthetic system 400 may the sensor-enabled prosthetic socket 100 described previously, along with a smart phone 86, a laptop computer 90, a data server 88, software 96 (algorithms, macros, computer learning and/or other data processing software). Many of the communications between these components of the system 400 have been described in relation to other embodiments and will not be repeated here. In this embodiment, the smart socket 100 includes a motorized closure system 530, which may be controlled automatically to adjust the socket 100. The communications between sensors 51, 56 and/or others on the socket 100 and the motorized closure system 530 may occur via two general paths—an on-board loop 410, which occurs on the socket 100, and an off-board loop 402 which occurs partially off the socket 100. In the on-board loop 410, sensors 51, 56 talk to the microprocessor/microcontroller/battery 59, which uses macros and internal presets 415 to provide instructions to the motorized closure system 530, which then actuates 404 the adjustment of the socket 100. In the off-board loop 402, sensor data is transmitted 82 to the smart phone 86, which processes the data to provide instructions 406 back to the motorized closure system 530, which then actuates 404 the adjustment of the socket 100. Alternatively, the off-board loop 402 may use the laptop computer 90 or any other suitable computing device with a processor.

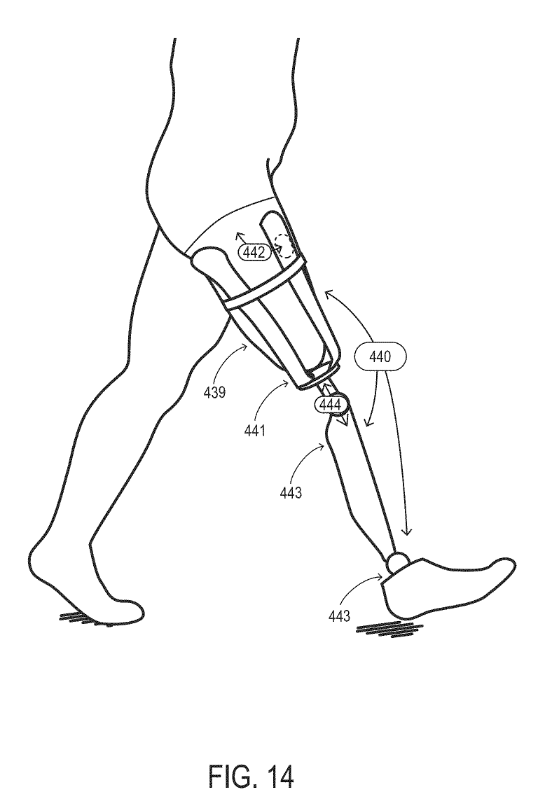

Referring now to FIG. 14, as mentioned previously, a prosthetic socket 441 is only one part of a prosthetic limb. The socket 441 is attached to one or more distal prosthetic components 443 to create the full limb, such as a knee, tibia, ankle and foot as illustrated in FIG. 14. In some embodiments, the smart prosthetic socket 441 may be part of a system that communicates with one or more of the distal components 443, for example via sensors on the various components. In some embodiments, the components may communicate wirelessly 440 across a distance between two separate components. Alternatively or additionally, the components may communicate directly 444, possibly through a wired connection, between the distal base of the prosthetic socket 441 and the proximal end of the distal components 443, for example. The smart prosthetic socket 441 may also communicate 442 with a smart liner 439 worn on the patient's residual limb in some embodiments. In addition to data, other resources may be shared between the socket 441 and the distal components 443 and/or smart liner 439, such as but not limited to electrical power, pneumatic pressure, hydraulic pressure, and/or other resources. Again, this transfer may happen by way of direct hardware or wire connection 444 or by way of wireless transfer 440. This data, electrical power, pneumatic pressure, hydraulic pressure, or other resource sharing is clinically significant in that this allows for a greater capacity to predict and react to negative events, such as falls.

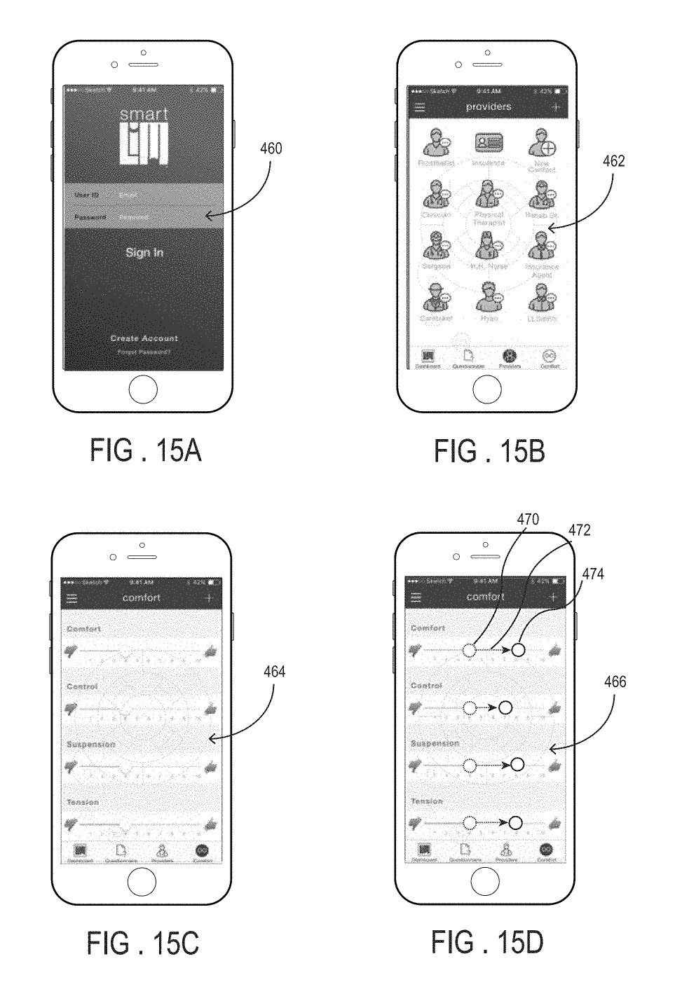

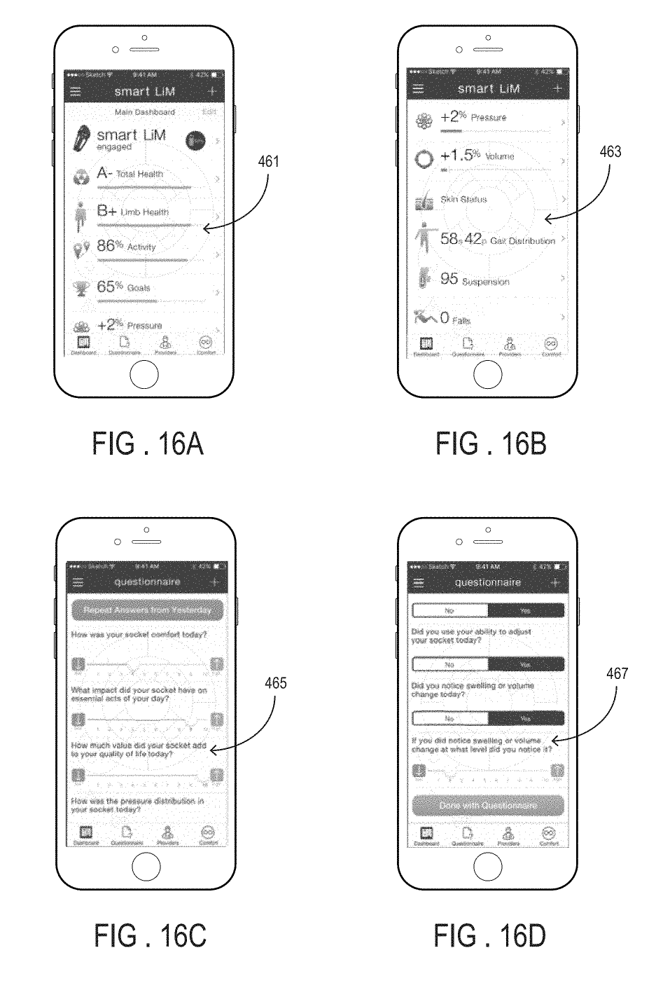

FIG. 15A-16D are screen shots illustrating one embodiment of a user interface for a smart phone application for a smart prosthetic socket system, including examples of how qualitative data can be collected from users and used to direct the user from an undesired socket state to a target socket state. FIG. 15A shows a user interface 460 for a smart phone application, including a user login, which is important for protecting the security of patient related information and for compliance with Health Insurance Portability and Accountability Act (HIPAA) regulations. FIG. 15B shows another user interface 462 for the smart phone application, which includes a service provider platform that connects the user with service provider options for meeting their prosthetic care and related health service requirements. This is one example of the smart phone application acting as a resource and service connection device. Having a resource database linked with the user profile and usage data details of a given user facilitates care capacities beyond typical and prior service provider platforms.

FIG. 15C shows another user interface 464 for the smart phone application, which includes a subjective rating input interface for the user to input information related to his or her socket state experience. When this rating is reported by the user, the socket state at that moment and leading up to that moment will be recorded, and software can process that data in a way that informs future recommendations, notifications, alerts, adjustments and/or the like. FIG. 15D shows another user interface 466, which also includes a subjective rating input interface, but in this example the ratings reported by the user have increased. Significant changes in user experience are variables that can be analyzed for differences and can potentially yield sources or causes for the changes. The interface 466 may include a toggle 470 that may be moved 472 to various different positions 474 to indicate changes.

FIG. 16A shows another user interface 461 for the smart phone application, including a user dashboard that compares the total input data against goals and targets set by the user, as well as comparing against general performance standards. These comparisons may be designed in a way to motivate the user and support the user in achieving goals, such as to improve health and avoid pain. FIG. 16B illustrates another user interface 463, which may be reached, for example, by scrolling down on the smart phone screen from the interface 461 of FIG. 16A. This lower portion also includes information that may be used to motivate the patient/user. Additionally, the application interface may show a percentage of change from the target, to highlight deviations from the target to the user. FIGS. 16C and 16D show a first portion 465 and a second portion 467, respectively, of a user interface for the smart phone application, including, in this embodiment, a questionnaire with questions that may help calibrate user value and/or pain points, such that the data can be used in the most efficient and effective way to improve results.

FIGS. 17A and 17B illustrate an approach to profiling force distribution through a transfemoral prosthetic socket. FIG. 17A is a simplified top view of a transfemoral prosthetic socket 480, showing the orientation of the socket 480. FIG. 17B shows the residual limb of a patient with a transfemoral amputation 481 and the regions of the limb that correspond to the regions of the transfemoral prosthetic socket 480. Longitudinal regions of the residual limb 481, in this embodiment, include a proximal/brim region 1510, a proximal region of the long axis 1520, a distal region of the long axis 1530, and a distal end region 1540. In various embodiments, the proximal/brim region 1510 encompasses approximately 25-40% of the residual limb, the proximal region of the long axis 1520 encompasses approximately 20-25% of the residual limb, the distal region of the long axis 1530 encompasses approximately 20-25% of the residual limb, and the distal end region 1540 encompasses approximately 20-25% of the residual limb. In this embodiment, the three most proximal longitudinal regions 1510, 1520, 1530 are also divided into four cross-sectional regions—medial-anterior, lateral-anterior, medial-posterior, and lateral-posterior. These four cross-sectional regions correspond to four anatomical planes and also to the location of four longitudinal struts of a prosthetic socket, in some embodiments. The distal region 1540 is not divided into cross-sectional regions in this embodiment. Another region identified in this profile is an ischial seat region 1500. The described regions may be associated with clinical significance, sensor distribution, software associations, pattern recognition, and/or other data processing.

FIGS. 17C-17E are posterior, lateral and anterior views, respectively, of a lower extremity 1300 of a human, demonstrating the lower extremity anatomy. The lower extremity includes major muscle groups of the thigh: the quadriceps muscle group 1222, which includes rectus femoris, vastus lateralis, vastus medialis, and vastus intermedius, and the hamstring muscle group 1200, which includes biceps femoris, semitendinosus and semimembranosus. In an alternative embodiment for designating regional distinctions within a residual limb, the regions may be based on some or all of these muscle groups and/or individual muscles. Although FIGS. 17C-17E show non-amputated extremities, the muscle regions of a residual limb usually mimic the same anatomical regions. Regions based on muscle groups or other anatomical distinctions may be generic, sized, or custom determined through imaging and/or sensors. In other alternative embodiments, other regional distinction strategies may be used to improve clinical effect, sensor accuracy, metric accuracy, efficiency, software associations, pattern recognition, and/or other data processing.

Referring now to FIG. 17F, an anterior view of a transtibial residual limb 1000 is illustrated. FIG. 17G is an anterior view of identified regions of a transtibial residual limb 1000, according to one embodiment. In this embodiment, the regions include a proximal longitudinal region 1031, a longitudinal region between bisection of the patella and apex of the fibula head 1032, a longitudinal region between the apex of the fibula head and the distal end 1033, and a distal end 1034. The proximal longitudinal region 1031 may encompass approximately 25-40% of the residual limb, the longitudinal region between bisection of the patella and apex of the fibula head 1032 may encompass approximately 20-25% of the residual limb, the longitudinal region between the apex of the fibula head and the distal end 1033 may encompass approximately 20-25% of the residual limb, and the distal end region 1034 may encompass approximately 20-25% of the residual limb. The three most proximal longitudinal regions are also divided into four cross-sectional regions—medial-anterior, lateral-anterior, medial-posterior, and lateral-posterior. The regions that can be viewed from the front include; region 1010, region 1011, region 1014, region 1018, region 1020, and region 1022. For the transtibial residual limb 1000, there are also several specific and asymmetric anatomical regions of clinical significance that are viewed from the front, including the patella region 1021, the patellar-tendon region 1012, the tibial crest region 1013, the distal anterior region 1015, the distal end region 1016, the fibular shaft area 1017, and the fibular head 1019. The medial side 1030 and lateral side 1029 are also shown to orient the residual limb 1000.

FIG. 17H is a posterior view of the transtibial residual limb 1000, and FIG. 17I is a posterior view of identified regions of the transtibial residual limb 1000. The same regional distinction for profiling used in FIG. 17G is also used in this figure. The regions that can be viewed from the back include region 1041, region 1044, region 1042, and region 1043. The top two longitudinal sections (region 1041 and region 1044) span two longitudinal regions, because they exhibit similar clinical significance and relationship with the other regions. For the transtibial residual limb 1000, there are also several posterior asymmetric anatomical regions of clinical significance, including the lateral hamstrings region 1040, the medial hamstrings region 1045, the distal end region 1016, the fibular shaft area 1017, and the fibular head 1019. The medial side 1030 and lateral side 1029 are also shown to orient the residual limb 1000. These regions also may be generic, sized, or custom determined through imaging and/or sensors.

The total input data or any portion therein may be associated with these or other specific regions and the relationship between these regions, including a comparison of like data per region, which offers a clinical or user resource that can improve prosthetic care. One example of using data for specific regions and the relationships between the regions to improve prosthetic care for the user is a transtibial prosthesis that can loosen automatically when the user sits down. This may be accomplished by a transtibial prosthesis that includes sensors in the regions shown above in FIGS. 17G and 17I, as well as a microcontroller and data processing capabilities that allow the socket to recognize the pressure distribution pattern of sitting when there is a sustained (e.g., more than 5 seconds) peak pressure for regions 1041 and 1044 (posterior-proximal regions) as well as in regions 1014, 1015, and 1018 (anterior-distal regions). This pattern can then trigger an automated mechanism to loosen the fit of the socket in order to provide more comfort. The associated pattern may also trigger user history within the database that warns the user that they had pain after the last time they were seated in this way for 10 minutes longer than their current sitting time. The user interface may provide a notification based on this data processing that helps the user avoid pain.

Another example of using data for specific regions and the relationships between the regions to improve prosthetic care for the user is a transtibial prosthetic socket that includes sensors in the regions shown above in FIGS. 17G and 17I, as well as a microcontroller and data processing capabilities that allow the socket to recognize that the user has just stumbled. This may be provided by recognizing a regionally specific pattern of a rapid and un-sustained spike in pressure for regions 1041 and 1044 (posterior-proximal regions) and in regions 1014, 1015, and 1018 (anterior-distal regions). The socket and software may then react by recording the totality of data leading up to that stumble for future prevention, analysis, and benefit, and/or the socket may deploy a knee guard to protect the patella and knee, in case the user falls from the stumble. The socket may also communicate with a smart knee to react appropriately and avoid or reduce the risk of injury.

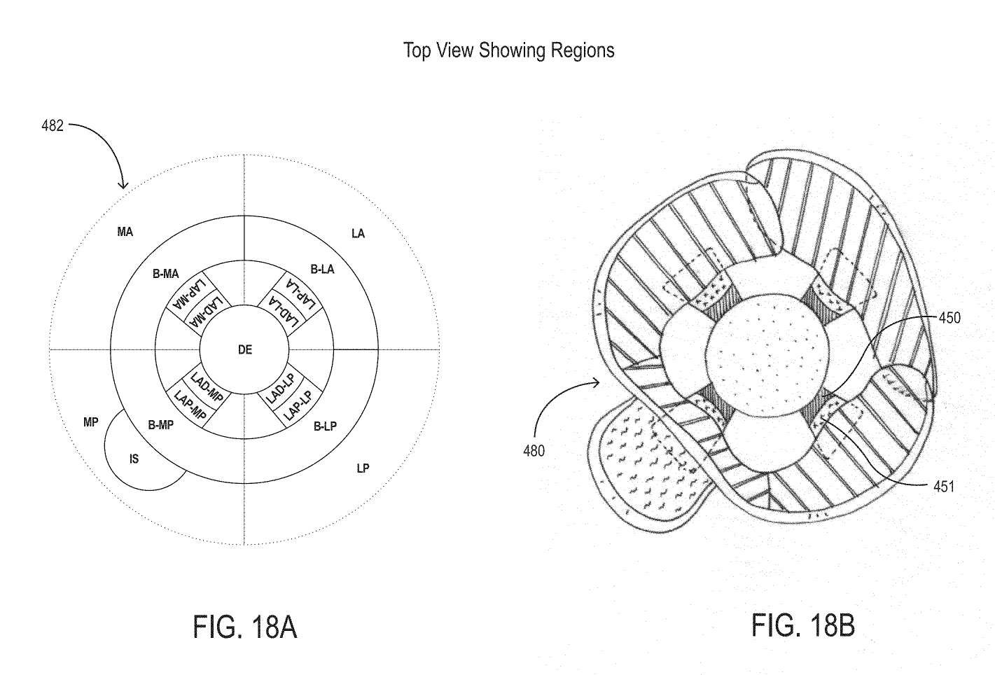

FIG. 18A shows a top view schematic representation of the transfemoral prosthetic socket 480 divided into the longitudinal regions described in FIG. 17B—the proximal or brim region (B), the long axis proximal region (LAP), the long axis distal region (LAD), and the distal end (DE). The three most proximal longitudinal regions are also divided into four cross-sectional regions—medial-anterior (MA), lateral-anterior (LA), medial-posterior (MP), and lateral-posterior (LP). Again, the ischial seat region (IS) is a separate region.

FIG. 18B shows a top of the transfemoral prosthetic socket 480 with shaded sections showing the regional aspects of the prosthetic socket described above in FIG. 18A. For example, the long axis distal region 450 corresponds to the region marked LAD-LP in FIG. 18A. The long axis proximal region 451 corresponds to the region marked LAP-LP in FIG. 18A.

FIGS. 19A and 19B are partial cutaway views of a prosthetic socket 480, with FIG. 19A illustrating a first side view, with the two lateral struts removed, and FIG. 19B illustrating a second side view, with the two medial struts removed. These two figures both show the different regions, including the long axis distal region 450 and the long axis proximal region 451, described above.

FIG. 20A shows a user interface dashboard image of a prosthetic socket schematic 486, according to one embodiment. The schematic 486 shows a hypothetical optimal distribution of force that represents an optimal socket state, including the desired amount of distal end pressure 455 and ischial seat pressure 456, as determined for an individual patient, in which the dashboard figure is reporting the force distribution in units of relative percentages of the total force through the socket. In this case, the relative distribution of force is represented by shades of grey rather than in numerical units, but alternative embodiments may additionally or alternatively include numerical values of force or ratings.

FIG. 20B shows another user interface dashboard image of a prosthetic socket schematic 487, indicating sampled force distribution of a hypothetical patient. The force distribution deviates from that of the optimal socket schematic 486. For example, the dark shading of the ischial seat region 458 on the schematic 487 indicates that the patient's residual limb is exerting too much force on the ischial seat of the prosthetic socket (and vice versa), and not enough force is being absorbed by the distal end 457 of the residual limb relative to the target values entered by the user.

FIG. 20C shows another user interface dashboard image of a prosthetic socket schematic 488, indicating the differences between an optimal distribution of force (per FIG. 20A) and a sampled distribution of force (per FIG. 20B) shown numerically as greater than optimal (+values) or less than optimal (−values) in relative percentages, including +50 percent shown at the ischial seat region 476 and −9 percent shown at the distal end 475.

FIG. 20D shows a user interface dashboard image of a prosthetic socket schematic 489, indicating the correction needed to resolve differences between the sampled force distribution values and the optimal distribution values as downward arrows (indicating the force needs to be corrected downward) and upward arrows (indicating the force needs to be corrected upward). In this embodiment, the thickness of the arrows correlates with the relative level of correction needed, for example a wide down arrow at the ischial seat region 458 and a medium up arrow at the distal end 457.

FIG. 20E is the same optimal force profile prosthetic socket schematic 486 of FIG. 20A. FIG. 20F shows another user interface dashboard image of a prosthetic socket schematic 491, indicating sampled force distribution that deviates from the optimal socket state (as in FIG. 20E). In this case, not enough force is absorbed by the ischial seat 478, and too much force is absorbed by the distal end 477 of the residual limb relative to the target values.

FIG. 20G shows another user interface dashboard image of a prosthetic socket schematic 492, indicating the differences between an optimal distribution of force (per FIG. 20E) and a sampled distribution of force (per FIG. 20F). The differences are shown numerically as greater than optimal (+values) or less than optimal (−values) in relative percentage terms, such as −18 percent shown at the ischial seat region 446 and +60 percent shown at the distal end 445.

FIG. 20H shows another user interface dashboard image of a prosthetic socket schematic 493, indicating the corrections needed to resolve differences between the sampled force distribution values and the optimal distribution values. The differences are expressed by downward arrows (indicating the force needs to be corrected downward) and upward arrows (indicating the force needs to be corrected upward). The thickness of the arrows correlates with the relative level of correction needed, for example a medium arrow at the ischial seat regions 478 and a wide down arrow at the distal end 477.

The regions of a prosthetic socket and residual limb specified in FIGS. 17A-20H are significant in terms of biomechanical control of the prosthesis. Other region designations may be used for transfemoral amputations, transtibial amputations or other amputation levels. The portions of the regions, as well as the locations of the regions, may be different in different embodiments. In many embodiments, 8-12 regions are designated, to provide a desired amount of detail needed for clinical significance without adding too many data points. Fewer regions may be designated for smaller residual limbs, and more regions may be designated for other residual limbs. The size of each region may vary, according to different embodiments, for example between about 6 cubic inches and about 24 cubic inches.

FIG. 21 shows a user interface dashboard or graphical display 494 of a user's 497 weight bearing distribution, including the amount of time spent on the sound side 496 (the non-prosthetic side) and on the prosthetic side 495, during ambulation. This information can be gathered through one or more of the sensors build into a sensor-enabled prosthetic socket coupled with software that facilitates pattern recognition of sound side vs. prosthetic side weight distribution during gait. This pattern recognition may be made more accurate or supported by integrated or third party data coming from outside the socket. For example, software may use data from a smart foot orthotic or shoe inserts on the sound side. This information may be relevant to the user and clinician for several reasons, including a correlation between back pain and gait symmetry, as well as prosthetic discomfort and weight distribution. Graphical display 494 may help ensure that the data is easily absorbed and understood.

Referring now to FIG. 22A, an exploded view of another embodiment of a modular transfemoral sensor-enabled prosthetic socket 510 is illustrated. In this embodiment, the prosthetic socket includes a data collection and transmission system 515 and an automated closure system 530. FIG. 22A shows many of the same components previously described in FIG. 6A, which will not be described again here. The data collection and transmission system 515 may include wireless RFID pressure sensors 520 and the microcontroller/data transmission unit/battery 59 with integrated motion sensor or accelerometer 53. The data collection and transmission system 515 may use wireless communication and RFID to communicate with the smart socket system components. The automated closure system 530 may include a closure belt 483 with a free end 542 that is held with a retaining loop 540, and a motor-operated belt advancement buckle 484 coupled with the closure belt 483 and including an on/off switch 532 and charging port 531.

FIG. 22B is a close-up view of the automated closure system 530 of FIG. 22A. In addition to what was shown and described in FIG. 22A, this detailed perspective shows an axle 538, a strain gauge 534, and an integrated sensor 533.

FIG. 22C is a side, cross-sectional view of the automated closure system 530 of FIGS. 22A and 22B. The motor-operated belt advancement buckle 484 is configured to advance the closure belt 483, which may include a tread or mating surface 539. The motor-operated belt advancement buckle 484 automates advancement or tightening of the closure belt 483 by the use of a motor 644 and a drive gear 991 which turns a tread 990 including the mating surface 539. The mating surface 539 matches that of the closure belt 483 or other combination of surfaces that provide traction, whereby the closure belt 483 is advanced through the buckle 484 as the tread 990 is turned counterclockwise. The motor-operated belt advancement buckle 484 also includes a release channel 536 at the axle 538 of a passive gear 992, which allows the tread 990 to be manually disengaged away from the closure belt 483, thereby allowing for manual loosening of the belt 483 if needed. The closure belt 483 may be a one-piece unit or two belt segments, in different embodiments. Different parts of the closure belt 483 may include a fixed portion 554 secured to the motor-operated belt advancement buckle 484, an active portion 550 that is tightened and/or loosened by the motor 644, and the free end 542, which is the portion of the belt 483 that has passed through the motor-operated belt advancement buckle 484.

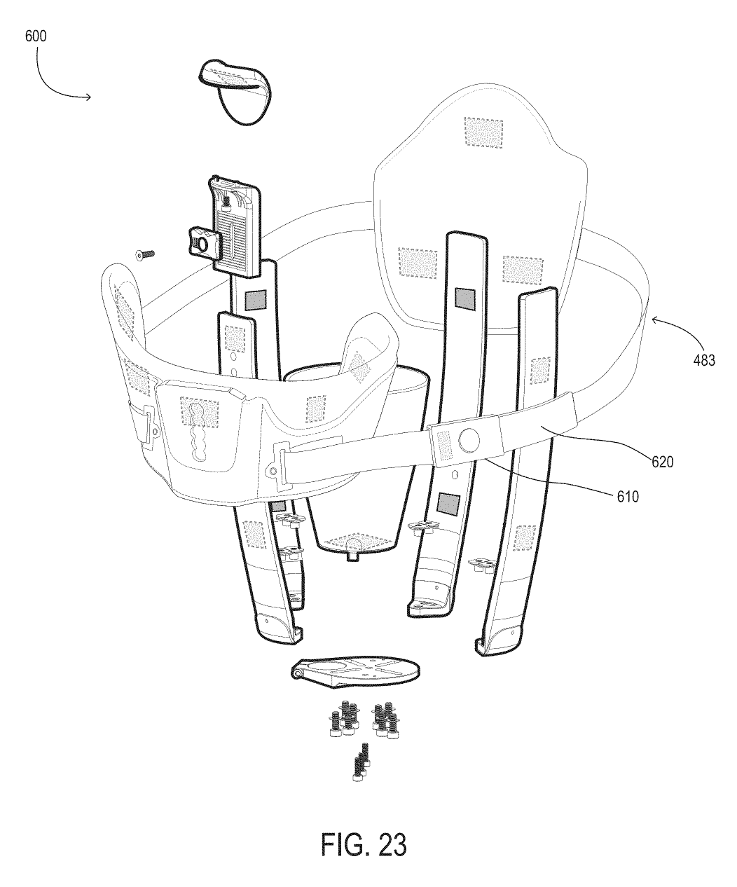

Referring now to FIG. 23, another embodiment of a modular transfemoral sensor-enabled prosthetic socket 600 is illustrated. This embodiment includes all of the features of the embodiment illustrated in FIG. 22A, except that it includes a different motor-operated belt closure system 610. In this embodiment of the motor-operated belt closure system 610, the free end of the closure belt 483 is housed within a housing 620. The housing 620 may be made of plastic or other suitable material and is configured to at least partially enclose a closure cable or closure belt 483.

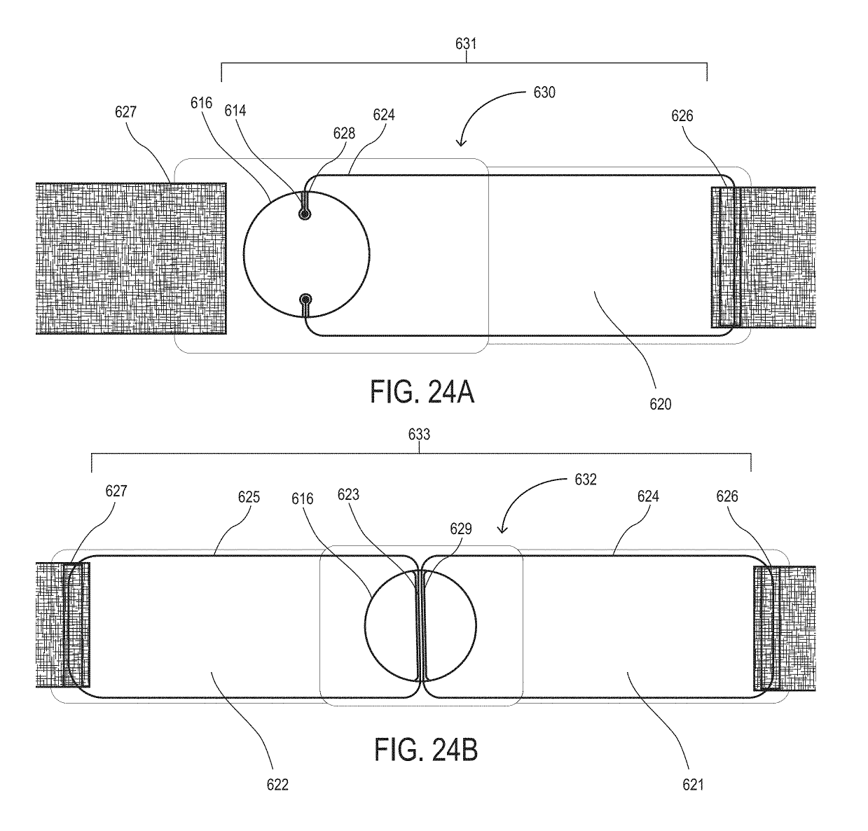

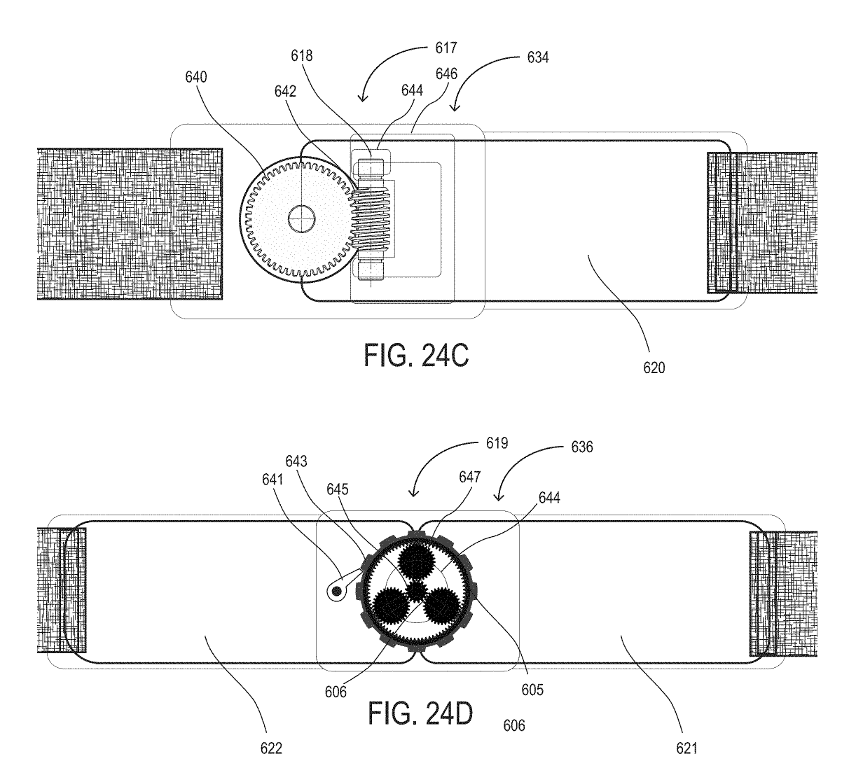

FIG. 24A-24D are front views of four different embodiments of a low-profile automated closure mechanism for an automatically adjustable or manually adjustable smart prosthetic socket. In these embodiments, the excess cable within the closure system is collected around a spool, thereby avoiding any loose or excess cable. The closure mechanisms shown here may be used as an automated or manual closure device.

Referring to FIG. 24A, one embodiment of an automated closure mechanism 630 includes a spool 616 to collect excess cable 624 as the closure mechanism 630 tightens, thereby shortening a distance 631 between a left side belt connection 627 and a right side belt connection 626 and thus effectively shortening the overall closure belt 483. The cable 624 includes a ball, stud, or shank end fitting 614 that mates with a cylindrical housing 628. The automated closure mechanism 630 includes one housing channel 620. Therefore, in this embodiment, the left side belt connection 627 and a right side belt connection 626 are shortened within the channel 620 when the closure mechanism 630 is tightened. Motorized mechanisms that allow for automated tightening or loosening of such the closure mechanism 630 may be included in some embodiments.

Referring to FIG. 24B, an alternative embodiment of an automated closure mechanism 632 includes some of the same features as the embodiment of FIG. 24A. This embodiment, however, includes a left housing channel 622 and a right housing channel 621, thereby providing closure forces or tightening forces on both sides of the closure belt as the spool 616 tightens clockwise to collect excess cable from a left side cable 624 and a right side cable 625 for closure mechanism 632. The cables 624 and 625, in this embodiment, are constructed and installed as a closed loop, where both loops are collected and secured at a juncture 623 within a channel 629 built into the spool 616. Collectively, these members shorten or lengthen a distance 633 between the left side belt connection 627 and a right side belt connection 626 as the spool 616 is tightened (clockwise turn in most motor or manual embodiments) or loosened (counter-clockwise turn in most motor or manual embodiments). Motorized mechanisms that allow for automated tightening or loosening of such the closure mechanism 632 may be included in some embodiments.

FIG. 24C shows the main internal components of an automated closure mechanism embodiment 634, with many of components described above in relation to FIG. 24A, but additionally including a worm gear system 617 that acts as an automated closure mechanism. The worm gear system 617 includes a worm wheel 640, a worm 642, a worm shaft 618, a motor 644, and a worm gear housing 646 (or “worm gear carrier”).

FIG. 24D shows the main internal components of an automated closure mechanism embodiment 636, with many of components described above in relation to FIG. 24B, but additionally showing a planetary gear system 619 that acts as an automated closure mechanism. The planetary gear system 619 includes a sun gear 645, a planetary gear 647, a ring gear 605, a motor 644, a carrier 606, an outside one-way locking gear 643, and a pawl 641. In alternative embodiments, the motor 644 may be placed alongside the worm gear system 619, with a motor belt transferring rotary forces to the sun gear 645 and may also be a one-way motor, where locking against loosening can be controlled by the outside one-way locking gear 643 and pawl 641.

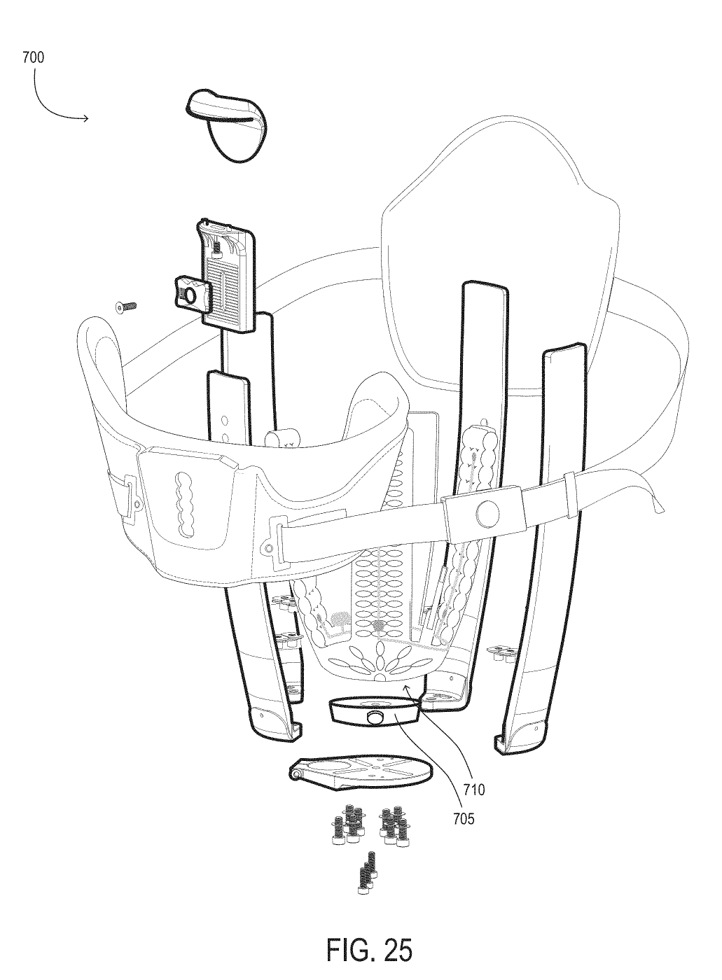

Referring now to FIG. 25, an exploded view of another embodiment of a modular transfemoral smart prosthetic socket 700 is illustrated. This figure shows the addition of a sensor-enabled pneumatic bladder system 710 and pin locking suspension system 705, which may be used an alternative suspension mechanism in this embodiment of the prosthetic socket 700.

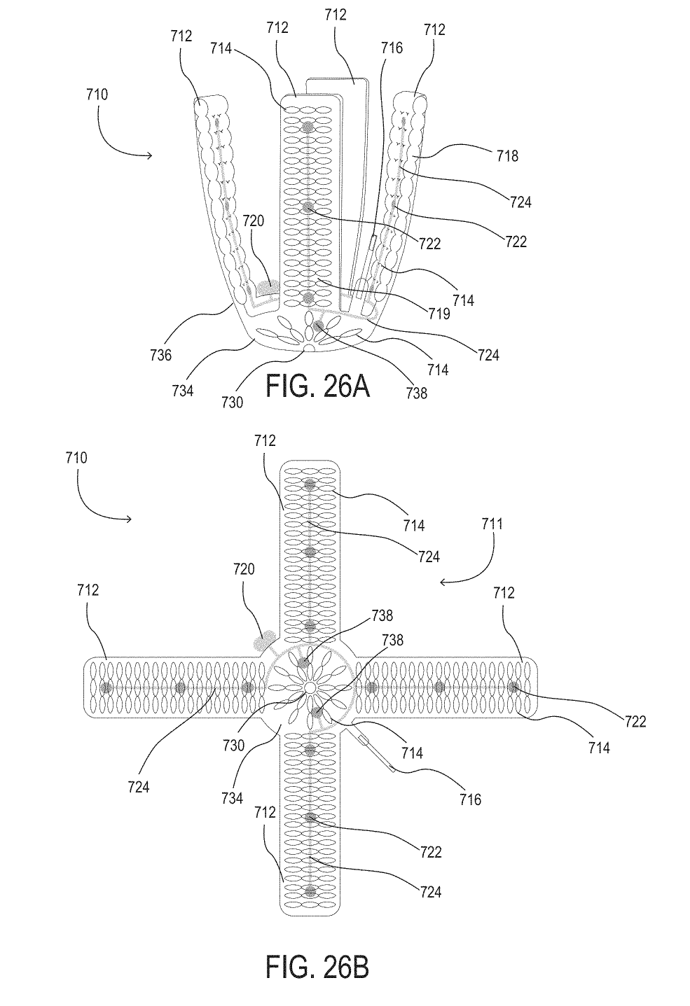

FIG. 26A is a perspective view of the sensor-enabled pneumatic bladder system 710 of FIG. 25. In this embodiment, the sensor-enabled pneumatic bladder system 710 includes a pneumatic bladder 719, longitudinal sections 712 (four in this embodiment, but other numbers are possible in alternative embodiments), a distal end section 734, and baffling 714 configured to shape the system 710 when inflated. In the embodiment shown, the baffling 714 forms the system 710 into a double-U cup shape 736 that is designed to cup around the distal end of the user's residual limb to provide increased biomechanical control and prevent the distal end of the residual limb from experiencing too much pressure as the side-walls 718 are inflated. The bladder system 710 also includes integrated sensors 722, distal end sensors 738 within the distal end section 734, conductive material 724 connecting the sensors 722, 738, a hole 730 within the distal end section 734 designed to allow passage of a pin (not shown) of the locking suspension system 705 (FIG. 25), a control center 720, and an emergency manual expulsion valve 716 in case of a dead battery or malfunction. The control center 720 is where the computing, transmission, and responsive aspects of capabilities may be located within the system. As such, the control center 720 may include one or more microcontrollers, microprocessors, batteries, valves, and/or a miniature pneumatic pump, according to some embodiments. The valve within the control center 720 may be a microprocessor/microprocessor controlled valve that can allow air in or out of the system or in an automated way with use of a microcontroller/microprocessor controlled valve and/or may actively pump air into or out of the system with the help of a miniature pneumatic pump.

The sensor-enabled pneumatic bladder system 710 may be set by the user, healthcare provider, and/or the others, to maintain a desired amount of average pressure regardless of volume fluctuation, to vary the amount of pressure per activity, to vary the amount of pressure over time or while sitting in order to help promote better circulation to the residual limb and body, and/or specific amounts of pressure per region of the residual limb or corresponding prosthesis.

FIG. 26B is a top view of the sensor-enabled pneumatic bladder system 710 of FIG. 25, as fabricated in a flat pattern 711. In some embodiments, the sensor-enabled pneumatic bladder system 710 may be manufactured as a flat piece, and the baffling 714 built into the system 710 causes it to assume its desired three-dimensional shape as air flows into the pneumatic bladder 719. All numbers of FIG. 26B are described in FIG. 26A and are simply shown here in a top view perspective of with the sensor-enabled pneumatic bladder system 710 flat.

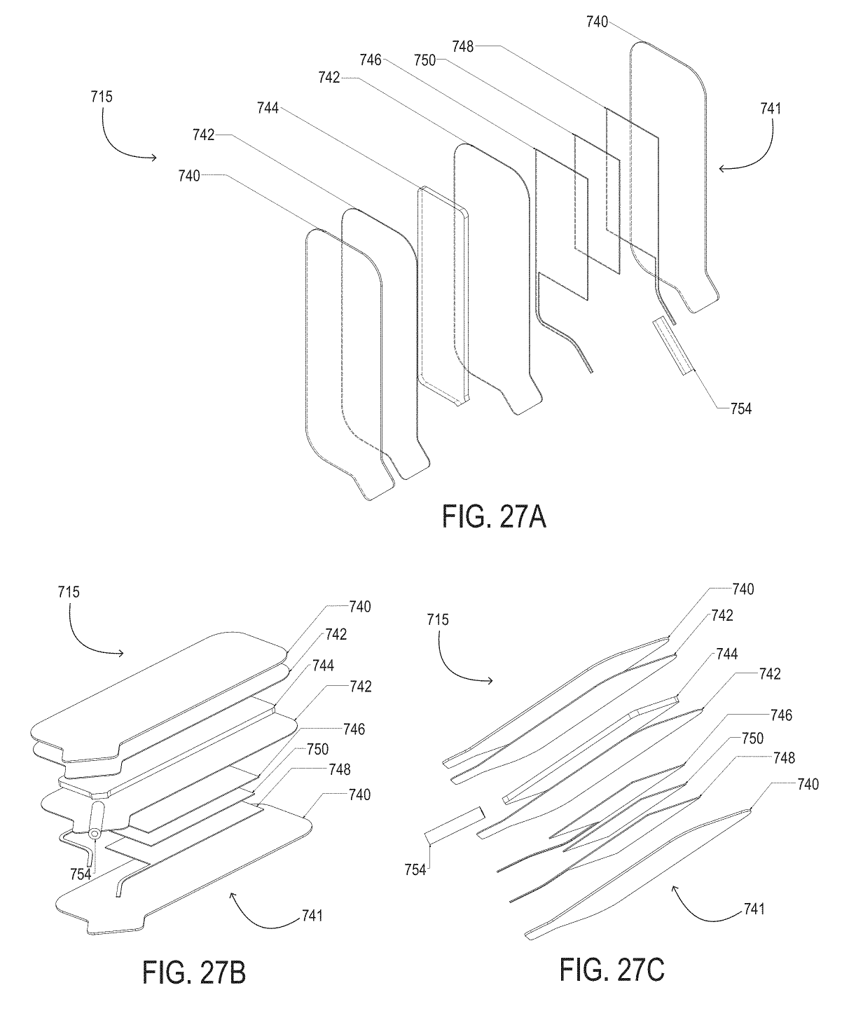

FIG. 27A-27C are three different perspective views of a material layup for fabrication of one embodiment of a sensor-enabled pneumatic bladder system 715. In this embodiment, the bladder system 715 includes a stretch woven fabric material 740, a thermoplastic polyurethane 742, a silicone foam 744 (approximately ⅛″ thick), an electronic ground 746 for the sensor (copper), a conductive material 748 (copper), and a polymeric foil 750 impregnated with carbon black (Velostat™) which is used because its resistance changes with either flexing or pressure. Also illustrated are the inside surface 741 of the bladder system 715 (e.g., the side closest to the residual limb of the user) and PVC tubing 754 that can be used to connect to a pump, valve, or other pneumatic control device, none of which is shown here.

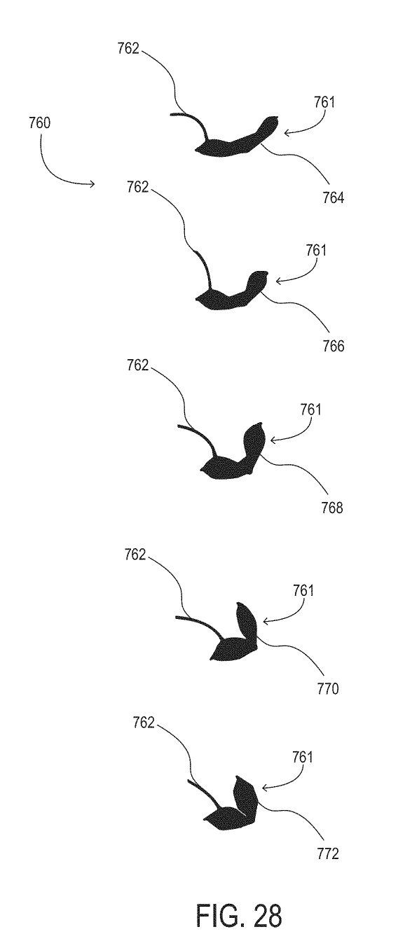

FIG. 28 shows a series of pneumatic bladder silhouettes 760, illustrating a change in shape of a pneumatic bladder 761 from a deflated to an inflated configuration. The bladder 761 includes an inflation/deflation tube 762 and is shown at the top of the series in a mostly deflated configuration 764 and then in increasingly more inflated configurations 766, 768, 770, in the descending silhouettes 760. The bottom frame illustrates the bladder 761 in its most inflated configuration 772. The shape of the bladder 761 may be controlled by the use of baffling (shown in previous figures) and/or other fabrication techniques.

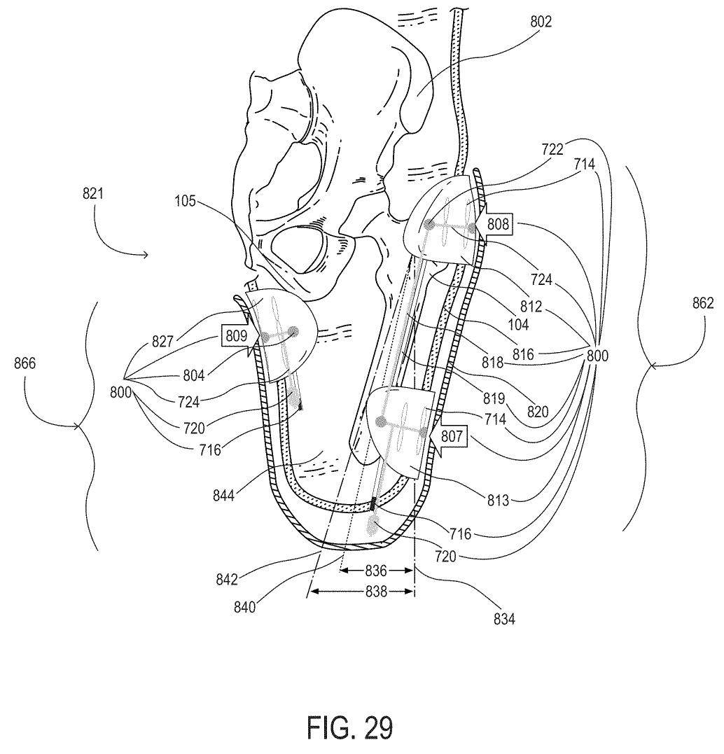

With reference now to FIG. 29, an anterior perspective cross-sectional view of a residual limb 844, inside another embodiment of a smart prosthetic socket 821 is illustrated. The prosthetic socket 821 includes a socket frame 820 and a sensor-enabled pneumatic bladder system 800, which work together with other components of the prosthetic socket 821 to increase biomechanical control of the femur 104 and residual limb 844. Three points of force are illustrated—a fulcrum force 809 and two counter-forces 807, 808. These forces 807, 808, 809 combine to adduct the femur 104 from a first angle 836 between the long axis of the femur 840 and a vertical axis 834 to a second angle 838 between the long axis of the femur 842 and the vertical axis 834.

FIG. 29 also shows the patient's ischium 105, pelvis 802 and skin 816, as anatomical references. Also illustrated are a medial pneumatic system 866, which in this embodiment includes a medial fulcrum pneumatic bladder 827, and a lateral pneumatic system 862, which in this embodiment includes a proximal-lateral counter-force pneumatic bladder 812 and a distal-lateral counter-force pneumatic bladder 813. A number of components from the embodiment of FIG. 26A are also included in this embodiment, and their descriptions will not be repeated. Additionally, a conductive material 818 joins one or more sensors within a pneumatic bladder to one or more other sensors, pneumatic bladders, microcontrollers, batteries, transmission devices, valves, miniature pumps, and/or other sensors. A channel 819 extends between two or more pneumatic bladders or from a bladder to a and emergency manual expulsion valve 716 to allow air to flow between bladders and/or to pass air into the system or out of the system.

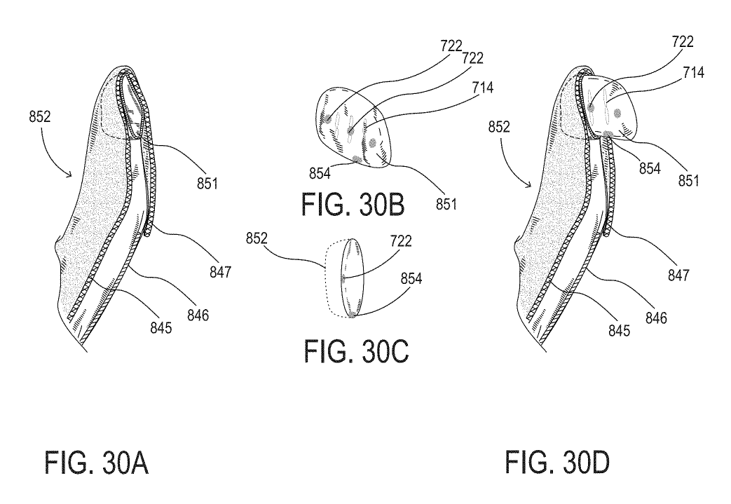

FIG. 30A is a perspective view of a quarter section of the proximal-lateral and posterior section of a prosthetic socket, according to another embodiment, where a textile sling 852 is disposed around the socket frame 846. The textile sling 852 has an inside textile layer 845 and an outside textile layer 847. In this embodiment, a smart pneumatic bladder 851 (shown in cross-section) is coupled with the textile sling 852.

FIG. 30B is a perspective view of the smart pneumatic bladder 851 of FIG. 30A, illustrating the placement of sensors 722 and baffling 714. In this embodiment, the sensors 722 are independent and connected wirelessly with RFID signals or other forms of transmission. This embodiment also includes a control center 854, which may include a microcontroller, a battery, a one-way pneumatic valve and/or miniature pneumatic pump, for example.

FIG. 30C is a side, cross-sectional view of the smart pneumatic bladder 851, where the dotted line 852 represents the sides of the smart pneumatic bladder 851 as it inflates and begins to take an intentional shape resulting from the baffling 714 and designed to cup around the trochanter (proximal section of the residual femur 104 shown in previous figures).

FIG. 30D is the same perspective view as in FIG. 30A but with the entire smart pneumatic bladder 851 illustrated and protruding out of the quarter section of the proximal-lateral and posterior section of a prosthetic socket 850.

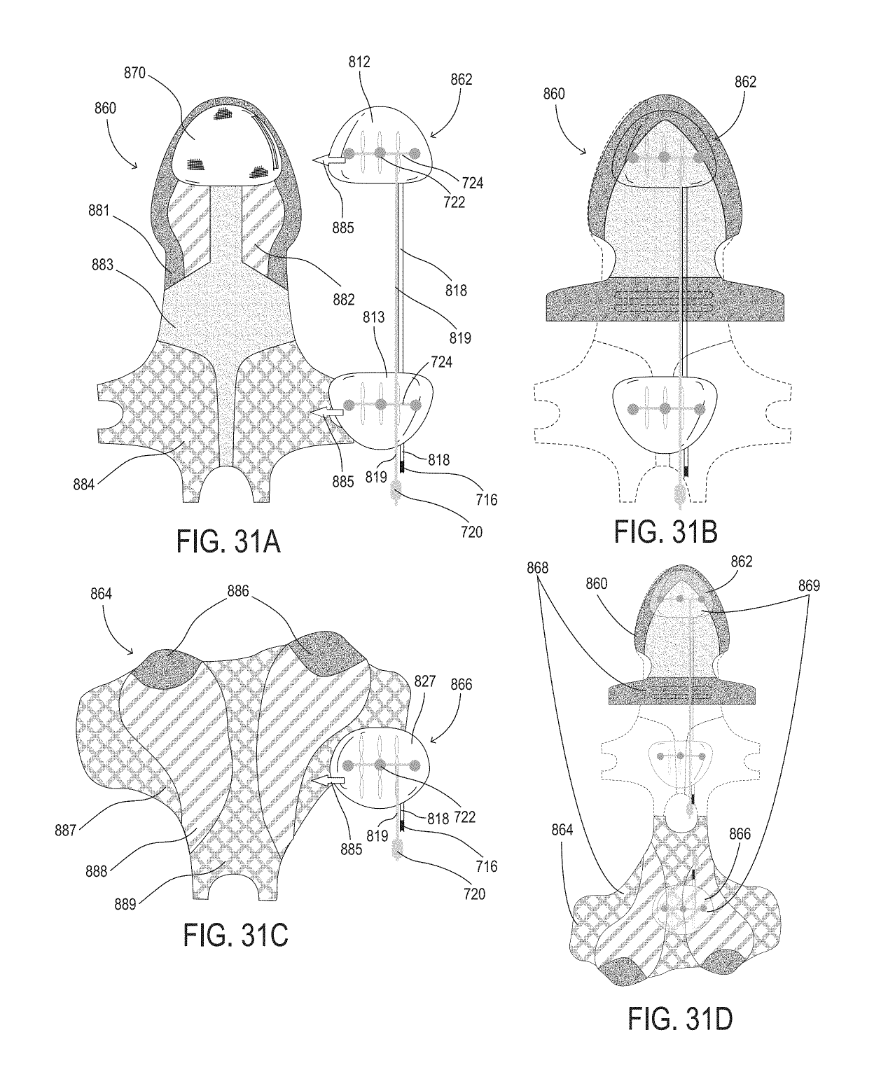

FIG. 31A-31D show a set of textiles slings with an integrated sensor-enabled pneumatic bladder system 869, which are made to work with a transfemoral prosthetic socket. FIG. 31A shows a lateral textile sling 860 configured for the lateral aspect of the socket, with a lateral pneumatic system 862. FIG. 31B shows the lateral textile sling 860 and the lateral pneumatic system 862 integrated together. FIG. 31C shows an outside surface of a medial textile sling 864 configured for the medial aspect of the socket, with a medial pneumatic system 866. FIG. 31D shows the lateral textile sling 860 coupled with the medial textile sling 864 to form a complete textile sling system 868 with an integrated smart pneumatic system 869.

Referring to FIG. 31A, the lateral textile sling 860 may include a pocket 870 for the lateral pneumatic system 862 and multiple pieces of textile fabric 881, 882, 883 and 884. The lateral pneumatic system 862 may include a first bladder 812 and a second bladder 813, which may be similar to or the same as the bladder 851 described in relation to FIGS. 30A-30D. The lateral pneumatic system 862 may also include a control center 720 and an emergency manual expulsion valve 716, which are described above in relation to FIG. 29. The bladders 812, 813 may be positioned over the lateral textile sling 860, as indicated by the hollow arrows pointing from the bladders 812, 813 toward the sling 860. FIG. 31B shows the lateral textile sling 860 coupled with the lateral pneumatic system 862.

Referring now to FIG. 31C, the medial textile sling 864 may include multiple pieces of textile fabric 886, 887, 888, 889. The medial pneumatic system 866 may include a medial bladder 827, as well as sensors 722, a control center 720 and an emergency manual expulsion valve 716, which are described above in relation to FIG. 29. The medial bladder 827 may be positioned over the medial textile sling 864, as indicated by the hollow arrow pointing from the medial bladder 827 toward the sling 864. FIG. 31D shows the lateral textile sling 860 and medial textile sling 864 coupled together to form to form the complete textile sling system 868 with the integrated smart pneumatic system 869.

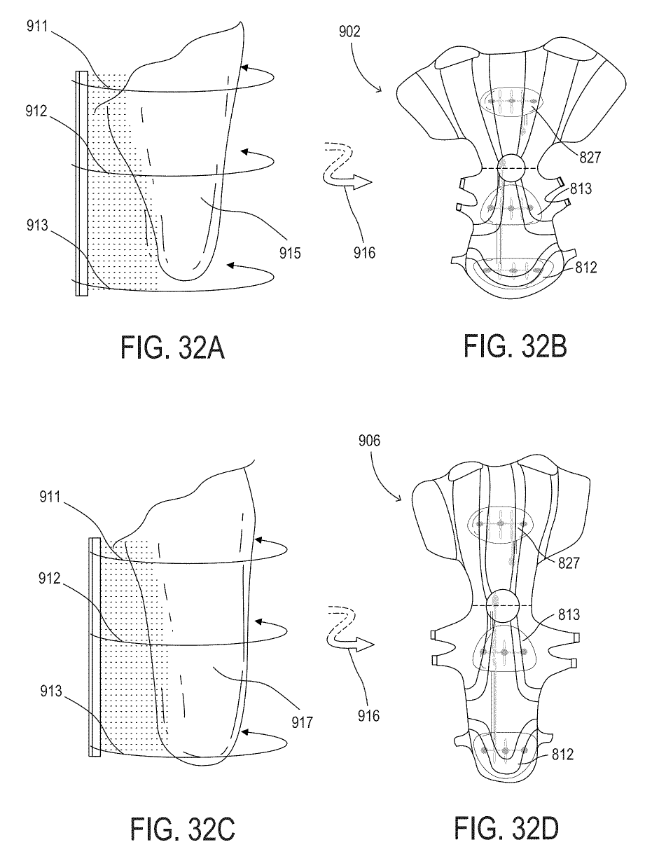

FIG. 32A shows a residual limb 915, which has different circumferences 911, 912, 913 at different longitudinal levels of the residual limb 915. As illustrated in FIG. 32B, these different circumferences 911, 912, 913 of the residual limb 915 may be used 916 to produce a custom-sized textile sling system 902, with an integrated smart pneumatic system as described previously.

FIG. 32A shows a different residual limb 917 which has different circumferences 911, 912, 913 at different longitudinal levels of the residual limb 917. As illustrated in FIG. 32D, these different circumferences 911, 912, 913 of the residual limb 917 may be used 916 to produce a custom-sized textile sling system 906, with an integrated smart pneumatic system as described previously.

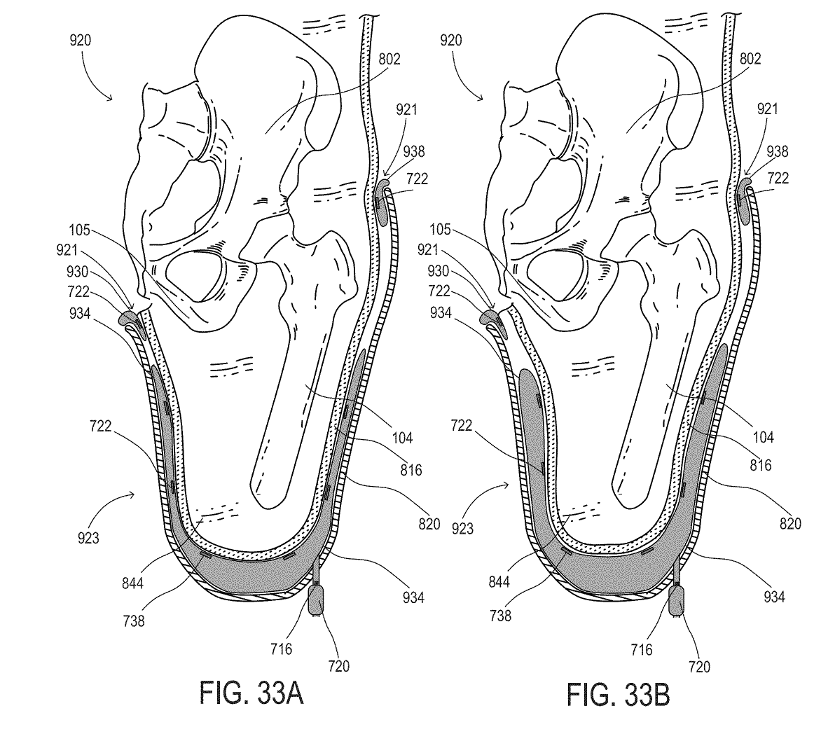

FIGS. 33A and 33B show a prosthetic socket system 920 with sensor-enabled pneumatic bladder system 923 that is used to manage volume change and/or intentionally vary the amount of pressure on the skin 816 of the residual limb 844. FIG. 33A shows the sensor-enabled pneumatic bladder system having less volume in the main pneumatic bladder 934 than in FIG. 33B. This change in volume can reflect a reduction in residual limb volume, with a maintained amount of pressure applied by the sensor-enabled pneumatic bladder system 923 onto the residual limb 844 or an increase in pressure applied by the sensor-enabled pneumatic bladder system onto the residual limb 844.

FIGS. 33A and 33B also include ischium 105, pelvis 802, residual femur 104, proximal brim smart pneumatic bladder system 921 with medial side 930 and lateral side 938 and integrated sensors 722, distal sensors 738, socket frame 820, and a control center 720 (including, for example, one or more microcontrollers/microprocessors, batteries, valves, and/or miniature pneumatic pump) and emergency manual expulsion valve 716 provided in case of a dead battery or malfunction. The proximal brim smart pneumatic bladder system 921 is similar to the other smart bladders described above, but this embodiment is specifically configured to protect the brim or proximal edges of the prosthetic socket.

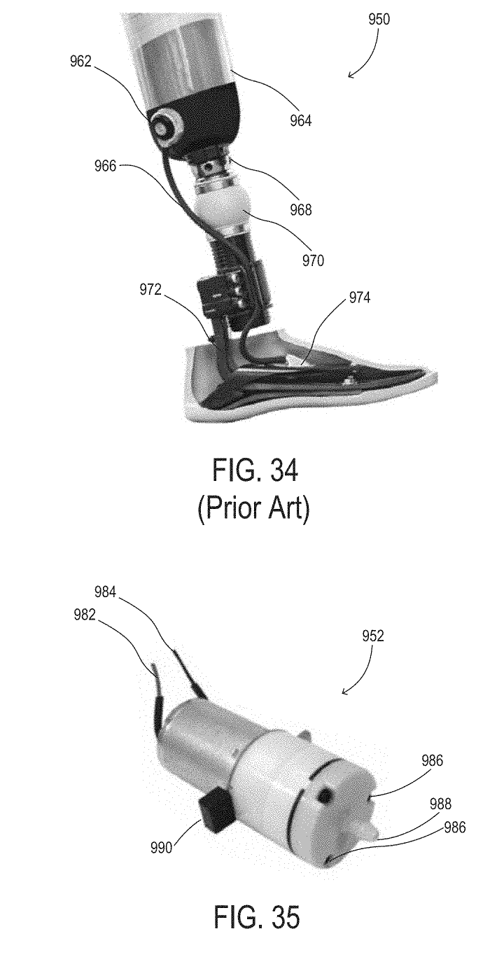

FIG. 34 shows an example of a pneumatic suspension and evacuation system 950 describe in prior art that uses kinetic energy of ambulation to evacuate air out of the system 950. The pneumatic suspension and evacuation system 950 includes a valve 962, a tube 966, a transtibial prosthetic socket 964, distal adaptors 968, a shock absorption unit 970, a prosthetic foot 972, and a kinetic energy pump 974. This system or similar systems may be used in conjunction with the sensor-enabled pneumatic bladder systems described herein, to help inflate and/or deflate the system.

FIG. 35 shows an example of a miniature pneumatic pump 952 which may be used to inflate and/or deflate the sensor-enabled pneumatic bladder systems described herein. Power for the pump 952 may be stored on or within the prosthetic socket or from an outside source. FIG. 35 shows a positive conductive wire 982 and a negative conductive wire 984 for battery current and/or charging, a microcontroller 990, an air expulsion port 988, and a motor vent 986. This type of pump 952 is one example of how a smart pneumatic system may inflate and/or deflate integrated smart pneumatic bladders.

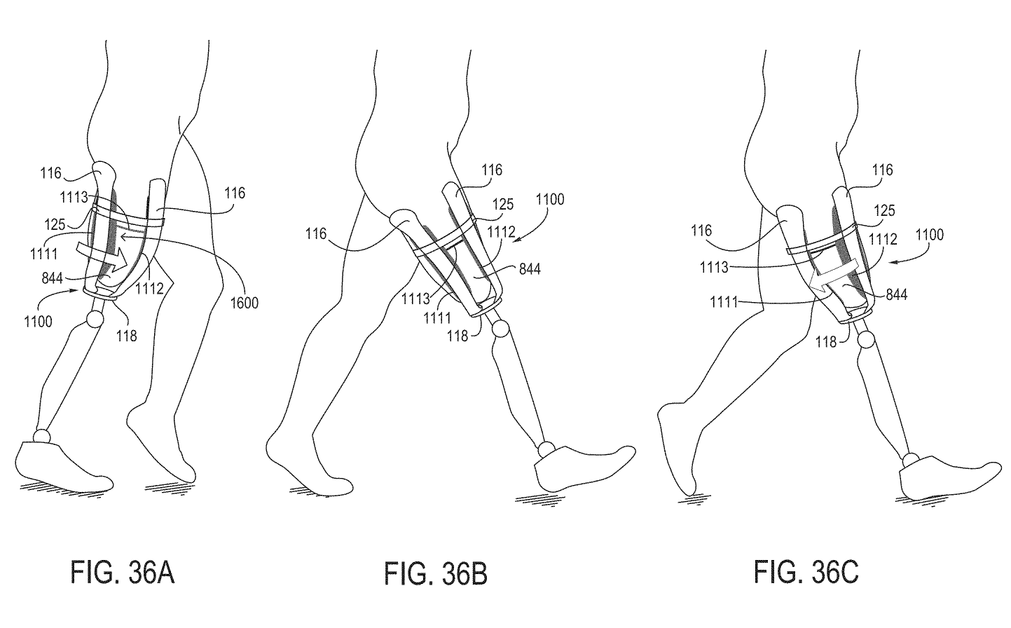

FIGS. 36A-36C are side views of a lower half of a patient with a residual limb 844 wearing a smart prosthetic socket 1100, illustrating movement of the residual limb 844 and the prosthetic socket 1100 during different phases of gait (walking). The figures demonstrate a reaction and accommodation of the movement by a smart bladder system 1600 integrated into the socket 1100. These figures demonstrate how this embodiment of smart bladder system 1600 shifts air between posterior bladders 1111 and anterior bladders 1112, by passing air through a connecting tube or channel 1113. The channel 1113 may be connected to (or include) a control center (not shown), to control the flow of air and sensors to detect force on the air bladder surface and/or air pressure within the air bladder segments or system 1600.

FIG. 36A is a side view of the residual limb 844 and the prosthetic socket 1100 (including struts 116, closure belt 125, and distal base 118), during the terminal stance phase of gait. In this phase, the residual limb 844 begins to flex forward, in order to initiate the swing phase of gait. As a result of the flexion moment of the residual limb 844, added pressure is applied to the anterior-distal aspect of the socket 1100, and pressure is reduced in the posterior-distal aspect of the socket 1100. The smart bladder system 1600 may accommodate for this change in pressure distribution, by transferring air from the anterior bladders 1112 to the posterior bladders 1111 through the connecting channel 1113. This action helps to equalize or more evenly distribute the pressure between the front and back of the socket 1100. By transferring air from one bladder to another, the system 1600 can be more energy efficient than it would be if it pumped air into and out of the system.

FIG. 36B shows the residual limb 844 in the terminal swing phase of gait, in which pressure is approximately equal from front to back, so the anterior bladders 1112 and posterior bladders 1111 are relatively equal in their pressure distribution. FIG. 36C shows residual limb 844 in the initial contact phase of gait, in which the residual limb 844 has an extension moment that adds pressure in the posterior-distal aspect of the socket 1100 and reduced pressure in the anterior-distal aspect of the socket 1100. The smart bladder system 1600 may accommodate for this change in pressure distribution by transferring air from the posterior bladders 1111 to the anterior bladders 1112 through the connecting channel 1113. This action helps to equalize or more evenly distribute the pressure between the front and back of the socket 1100 and adds comfort and control for the user.

What is claimed is:

1. A system for profiling a distribution of forces applied to a prosthetic socket by a residual limb of a wearer of the socket, the system comprising:
a prosthetic socket, comprising:
a longitudinal portion, comprising multiple struts;
a proximal portion, comprising at least one proximal brim member coupled with proximal ends of the multiple struts;
a distal portion, comprising a distal base coupled with distal ends of the multiple struts; and
an adjustment member coupled with at least one of the proximal portion, the longitudinal portion or the distal portion, configured to adjust the prosthetic socket to alter a force distribution profile within the prosthetic socket;
a sensor network comprising multiple sensors coupled with the prosthetic socket in a pattern defining multiple internal regions within the prosthetic socket; and
a processor coupled with the sensor network and configured to receive sensed data from the sensor network, divide the sensed data into groups corresponding to the multiple internal regions within the prosthetic socket, and process the sensed data to provide force distribution profile data corresponding to the force distribution profile.

2. The system of claim 1, wherein the processor is directly attached to the prosthetic socket.

3. The system of claim 1, wherein the processor is separate from and wirelessly coupled with the prosthetic socket.

4. The system of claim 3, wherein the processor is housed in a controller configured to allow the wearer of the prosthetic socket or another user to control at least one feature of the prosthetic socket.

5. The system of claim 1, wherein the processor comprises an off-socket processor separate from the prosthetic socket and configured to receive the sensed data from the prosthetic socket, divide the sensed data into the groups corresponding to the multiple internal regions within the prosthetic socket, and process the sensed data to provide the force distribution profile data, and wherein the system further comprises a microprocessor directly attached to the prosthetic socket and configured to receive the sensed data from the sensor network and wirelessly transmit the sensed data to the off-socket processor.

6. The system of claim 5, wherein the microprocessor is configured to perform initial processing of the sensed data before transmitting the sensed data to the off-socket processor.

7. The system of claim 5, wherein the off-socket processor is positioned in a location selected from the group consisting of a computer application on a mobile computing device, a tablet computer, a laptop computer, a desktop computer, a computer server and the cloud.

8. The system of claim 1, wherein the sensors of the sensor network are selected from the group consisting of a force sensor, a strain gauge, a Hall sensor, a flex sensor, a proximity sensor, a GPS, a flex sensor, a 3-axis accelerometer, a 3-axis gyroscope, and a 3-axis magnetometer.

9. The system of claim 1, wherein the multiple internal regions comprise:
a proximal region corresponding to the proximal portion of the prosthetic socket;
a longitudinal region corresponding to the longitudinal portion of the prosthetic socket; and
a distal region corresponding to the distal portion of the prosthetic socket.

10. The system of claim 9, wherein each of the proximal region, the longitudinal region and the distal region is further divided into four sub-regions, comprising:
a mediolateral region;
a medioposterior region;
a lateroanterior region; and
a lateroposterior region.

11. The system of claim 9, wherein the multiple internal regions further comprise an ischial seat region.

12. The system of claim 9, wherein the longitudinal region is further divided into an upper longitudinal region and a lower longitudinal region.

13. The system of claim 1, wherein the at least one proximal brim member comprises:
a lateral brim member; and
a medial brim member.

14. The system of claim 1, wherein the at least one proximal brim member comprises an ischial seat member.

15. The system of claim 1, wherein the force distribution profile data comprises multiple percentages of force applied by the residual limb to the prosthetic socket over the multiple internal regions.

16. The system of claim 1, wherein the force distribution profile describes force distribution through each of the multiple struts.

17. The system of claim 16, wherein the force distribution profile describes force distribution in:
a central path through a distal end of the residual limb; and
a peripheral path through the longitudinal portion of prosthetic socket.

18. The system of claim 1, wherein the processor is further configured to:
compare the force distribution profile data with a desired force distribution profile stored in a computer memory of the system; and
provide the wearer or another user with comparison data.

19. The system of claim 18, wherein the comparison data comprises an alert when the force distribution profile data is outside of a predetermined range of the desired force distribution profile data.

20. The system of claim 18, wherein the processor is further configured to:
receive user input from the wearer describing a desired fit of the prosthetic socket on the wearer's residual limb; and
generate the desired force distribution profile at least in part based upon the user input.

21. The system of claim 20, wherein the system further includes a computer application for a mobile computing device, and wherein the user input is provided by the wearer via the computer application.

22. The system of claim 18, wherein the processor is further configured to:
receive contralateral leg data regarding a contralateral leg of the wearer, on which a prosthetic is not being worn; and
use the contralateral leg data, at least in part, to generate the desired force distribution profile.

23. The system of claim 18, wherein the processor is further configured to generate a wearer wellness index based on the comparison of the force distribution profile data with the desired force distribution profile and at least one other factor describing the wearer or the prosthetic socket.

24. The system of claim 1, wherein the adjustment member comprises an adjustable hinge located between at least one of the distal ends of the struts and the distal base and configured to be fixed at a desired angle.

25. The system of claim 1, wherein the adjustment member comprises an adjustable height ischial seat member mounted on one of the proximal ends of one of the multiple struts.

26. The system of claim 1, wherein the adjustment member comprises:
at least one tensioning band coupled with the multiple struts; and
at least one tension adjustment member attached with each of the at least one tensioning bands.

27. The system of claim 1, wherein the adjustment member comprises a motorized closure mechanism configured to receive a command signal from the processor and automatically adjust the prosthetic socket based on the command signal.

28. The system of claim 27, wherein the motorized closure mechanism is further configured to maintain a tension in the prosthetic socket within a predetermined tension range.

29. The system of claim 1, wherein the adjustment member comprises a hinge mechanism configured receive a command signal from the control unit and automatically adjust the prosthetic socket based on the command signal.

30. The system of claim 1, wherein the sensor network further comprises at least one off-socket sensor configured to be attached to the wearer of the prosthetic socket at a location separate from the prosthetic socket.

31. The system of claim 1, further comprising a control unit coupled with the prosthetic socket, wherein the processor is housed within the control unit.

32. The system of claim 1, further comprising a control unit that is physically separate from the prosthetic socket, wherein the processor is housed within the control unit, and wherein the sensors are wirelessly coupled with the control unit.

33. A method for generating force distribution profile data for a prosthetic socket on a residual limb of a wearer of the socket, the method comprising:
sensing forces applied to multiple predefined regions of an inner surface of the prosthetic socket by the residual limb, using a sensor network attached to the prosthetic socket, wherein the sensor network comprises at least one sensor disposed in each of the predefined regions;
transmitting sensed data from the sensor network to a processor coupled with the sensor network;
processing the sensed data with the processor, wherein processing the sensed data comprises:
grouping the sensed data into multiple groups corresponding to the predefined regions; and
generating force distribution profile data for the prosthetic socket, based on the sensed data and the multiple groups.

34. The method of claim 33, further comprising providing the force distribution profile data to a user.

35. The method of claim 33, further comprising automatically adjusting the prosthetic socket, based on the force distribution profile data.

36. The method of claim 35, further comprising comparing the force distribution profile data to a desired force distribution profile, wherein the step of automatically adjusting the prosthetic socket is based at least in part on the comparing step.

37. The method of claim 36, further comprising providing an alert when the force distribution profile data is outside of a predetermined range of the desired force distribution profile data.

38. The method of claim 33, further comprising:
receiving user input from the wearer describing a desired fit of the prosthetic socket on the wearer's residual limb; and
generating a desired force distribution profile at least in part based upon the user input.

39. The method of claim 38, wherein the user input is received via a computer application on a mobile computing device.

40. The method of claim 33, further comprising:
receiving contralateral leg data regarding a contralateral leg of the wearer, on which a prosthetic is not being worn; and
using the contralateral leg data, at least in part, to generate a desired force distribution profile for the prosthetic socket.

41. The method of claim 33, further comprising generating a wearer wellness index based on the force distribution profile data and at least one other factor describing the wearer or the prosthetic socket.

42. The method of claim 33, further comprising:
sensing an acceleration of at least a portion of the prosthetic socket, using the sensor network; and
generating an acceleration distribution profile for the prosthetic socket, based on the sensed acceleration.

43. The method of claim 33, further comprising:
sensing a position of at least a portion of the prosthetic socket, using the sensor network; and
generating a position distribution profile for the prosthetic socket, based on the sensed position.

44. The method of claim 33, wherein the sensors of the sensor network are selected from the group consisting of a force sensor, a strain gauge, a Hall sensor, a flex sensor, a proximity sensor, a GPS, a flex sensor, a 3-axis accelerometer, a 3-axis gyroscope, and a 3-axis magnetometer.

45. The method of claim 33, wherein the multiple predefined regions comprise:
a proximal region corresponding to a proximal portion of the prosthetic socket;
a longitudinal region corresponding to a longitudinal portion of the prosthetic socket; and
a distal region corresponding to a distal portion of the prosthetic socket.

46. The method of claim 45, wherein each of the proximal region, the longitudinal region and the distal region is further divided into four sub-regions, comprising:
a mediolateral region;
a medioposterior region;
a lateroanterior region; and
a lateroposterior region.

47. The method of claim 45, wherein the multiple predefined regions further comprise an ischial seat region.

48. The method of claim 45, wherein the longitudinal region is further divided into an upper longitudinal region and a lower longitudinal region.

49. The method of claim 33, wherein the prosthetic socket comprises three or more struts, and wherein generating the force distribution profile data comprises comparing amounts of force in each of the struts.

50. The method of claim 49, wherein the prosthetic socket comprises a transfemoral prosthetic socket comprising four struts, wherein one of the struts is a medial-posterior strut, and wherein generating the force distribution profile data comprises comparing an amount of force delivered through the medial-posterior strut with amounts of forced delivered through the other three of the four struts.

51. The method of claim 33, wherein the prosthetic socket includes a microprocessor, and wherein the transmitting step comprises:
transmitting the sensed data from the sensor network to the microprocessor; and
transmitting the sensed data from the microprocessor to the processor, wherein the processor is located separately from the prosthetic socket.

52. The method of claim 51, further comprising conducting initial processing of the sensed data at the microprocessor before transmitting to the processor.

53. The method of claim 33, wherein the processor is located off of the prosthetic socket and is coupled wirelessly with the sensors of the sensor network.

54. The method of claim 33, wherein transmitting the sensed data comprises transmitting a location identifier from each of the multiple sensors.

55. The method of claim 33, further comprising displaying the force distribution profile data on a display device.

56. The method of claim 55, wherein the display device comprises a controller that is separate from the prosthetic socket.

57. The method of claim 56, wherein the processor is housed in the controller.

58. The method of claim 57, further comprising providing an alert on the controller when the force distribution profile data falls at least partially outside of a predetermined range of desired force distribution profile data.

59. The method of claim 33, further comprising adjusting tension in the prosthetic socket, using a motorized tensioning mechanism attached to the socket, based at least in part on the force distribution profile data.

60. The method of claim 59, wherein the tension is adjusted automatically.

61. The method of claim 33, further comprising automatically adjusting at least one characteristic of the prosthetic socket to adjust the force distribution profile data toward a desired force distribution profile.

62. The method of claim 61, wherein adjusting the force distribution profile data comprises moving a force distribution profile longitudinally within a length of the prosthetic socket.

63. The method of claim 61, wherein adjusting the force distribution profile data comprises moving a force distribution profile within a cross sectional anterior-posterior/lateral-medial grid within the prosthetic socket.

64. The method of claim 61, wherein adjusting the force distribution profile data comprises moving a force distribution profile comprises actuating a hinge mechanism within the prosthetic socket.

65. The method of claim 33, wherein the force distribution profile data are selected from the group consisting of a distribution of forces impinging on the prosthetic socket in relation to a central longitudinal axis of the socket, an absolute level of force applied by a distal end of the residual limb to the prosthetic socket, and a relative fraction of a total force applied by a distal end of the residual limb to the prosthetic socket.

66. The method of claim 33, further comprising:
testing the prosthetic socket on the residual limb; and
determining a desired force distribution profile for the socket and the residual limb.

67. The method of claim 66, wherein testing the prosthetic socket comprises:
placing the prosthetic socket on the residual limb; and
making initial adjustments to one or more mechanical features on the prosthetic socket.

68. The method of claim 33, further comprising repeating the sensing, transmitting and processing steps.

69. The method of claim 68, wherein the repeating is performed continuously over a period of time.

70. The method of claim 68, wherein the repeating is performed at set time intervals.
